(19) United States
(12) Patent Application Publication
(10) Pub. No.: US 2024/0317745 A1
(43) Pub. Date: Sep. 26, 2024

(54) SIGMA-1 RECEPTOR ANTAGONISTS AND THEIR APPLICATIONS

(71) Applicant: Humanwell Pharmaceutical US, Inc., St. Louis, MO (US)

(72) Inventors: Shuo Zhao, St. Louis, MO (US); Subo Liao, St. Louis, MO (US); Jun Yang, St. Louis, MO (US); Hao Zhou, Yichang (CN); Zejian Ding, Yichang (CN); Yao He, Yichang (CN); Xiongjun Mou, Yichang (CN); Youjian Ning, Yichang (CN); Xin Zheng, Yichang (CN)

(21) Appl. No.: 18/605,398

(22) Filed: Mar. 14, 2024

Related U.S. Application Data
(60) Provisional application No. 63/452,034, filed on Mar. 14, 2023.

(51) Int. Cl. C07D 471/04; A61K 31/5377; A61P 25/04; C07D 413/12

(52) U.S. Cl. CPC C07D 471/04; A61K 31/5377; A61P 25/04; C07D 413/12

(57) ABSTRACT

The present disclosure is directed to a class of Sigma-1 receptor small molecule antagonists, their pharmaceutical compositions, preparation methods, and uses. The Sigma-1 receptor small molecule antagonists are shown in formula (I), with specific substituents and definitions described in the specification. These Sigma-1 receptor small molecule antagonists exhibit good binding and antagonistic activity with the Sigma-1 receptor. The invention includes these compounds or their pharmaceutical compositions and their use in treating and/or preventing pain disorders related to the Sigma-1 receptor.

### CROSS REFERENCE TO RELATED APPLICATIONS

This application claims the benefit of U.S. Provisional Application No. 63/452,034, filed Mar. 14, 2023. The entire disclosure of which is hereby incorporated by reference.

### FIELD OF INVENTION

The current disclosure pertains to new small heterocyclic compounds designed as Sigma-1 receptor antagonists, intended to manage pain and associated conditions.

### BACKGROUND OF THE INVENTION

Pain relief medications are vital in enhancing the quality of life for patients suffering from various conditions. As the world's population ages and the prevalence of chronic illnesses like diabetes, arthritis, joint or bone pain, epilepsy, depression, nerve damage, and various forms of cancer rises, the demand for effective pain management solutions has grown significantly. Currently, the market for pain management treatments is primarily dominated by opioids and non-steroidal anti-inflammatory drugs (NSAIDs). However, the adverse effects associated with opioids, including respiratory depression, dependency, constipation, and the social issues stemming from their misuse, underscore the critical need for the development of non-addictive painkillers to address the pressing needs of a majority of patients.

The Sigma-1 (σ1) receptor, identified as a chaperone protein, plays a crucial role in modulating the activity of various proteins and ion channels, including N-methyl-D-aspartate (NMDA) and opioid receptors. Located in critical regions for pain regulation within both the peripheral and central nervous systems, the Sigma-1 receptor functions as a unique ligand-activated chaperone. Sigma-1 antagonists interfere with the receptor's chaperoning function, amplifying opioid signaling and reducing NMDAR activity. This dual action not only boosts opioid-induced pain relief but also mitigates the sensory hypersensitivity associated with chronic pain conditions. Significantly, targeting the Sigma-1 receptor does not exacerbate opioid-related side effects, suggesting that Sigma-1 antagonists could enhance the safety and efficacy of opioid therapies. Additionally, σ1 receptor antagonists show promise in addressing psychoses, pain, substance abuse, and cancer, highlighting their potential as versatile therapeutic agents.

It is known that in contrast to the seven transmembrane domains of opioid receptors, the Sigma-1 receptor is a single polypeptide composed by 223 amino acids, with only two transmembrane domains and no homology to opioid receptors or to any other known mammalian protein. The Sigma-1 (σ1) receptor protein is present within the mitochondria-associated endoplasmic reticulum (ER) membrane (Hayashi et al. Cell 131(3), 596-610, 2007). Sigma-1 (σ1) receptor is known to modulate Ca²⁺ signaling through the inositol trisphosphate receptor (IP3R) (Su et al. Trends Pharmacol. Sci. 31(12), 557-566, 2010) as well as hippocampal dendritic spine arborization through the regulation of reactive oxygen species (ROS) levels (Tsai et al. Proc. Natl. Acad. Sci. USA 106(52), 22468-22473, 2009). The crystal structure of the human σ1 receptor in complex with two ligands revealed a trimeric structure with a single transmembrane domain in each protomer (Schmidt et al. Nature 532 (7600), 527-530 (2016)).

Several small Sigma-1 (σ1) receptor antagonists have been reported in the literatures (Marrazzo et al. Life Sci. 78(21), 2449-2453, 2006; Moison et al. Neuropharmacology 45(7), 945-953, 2003; Marrazzo et al. J. Med. Chem. 54(10), 3669-3673, 2011; Parenti et al. Inflammation 37 (1), 261-266, 2014; Guitart et al. CNS Drug Rev. 4(3), 201-224, 1998; Cobos et al. Curr. Neuropharmacol. 6(4), 344-366, 2008; Lan et al. J. Med. Chem. 57(24), 10404-10423, 2014; Lan et al. Bioorg. Med. Chem. Lett. 26(8), 2051-2056, 2016; Diaz et al. J. Med. Chem. 55(19), 8211-8224, 2012; Berardi et al. J. Med. Chem. 48(26), 8237-8244, 2005). Patents have also been filed by pharmaceutical companies to protect their inventions on Sigma-1 (σ1) receptor antagonists including but not limited to US 2016/0060275, U.S. Pat. No. 7,696,199 B2, US 2008/0125416, and US 2016/0060275.

Herein, we disclose a series of novel Sigma-1 (σ1) receptor antagonists for the treatment of pain and related disorders.

### SUMMARY OF THE INVENTION

The following is an overview of the detailed description of the present invention. This summary is not intended to limit the scope of the claims.

The present disclosure is directed to a compound as shown in formula (I), or its stereoisomers, pharmaceutically acceptable salts, solvates, deuterated derivatives, metabolites, or prodrugs;

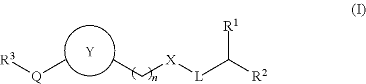

wherein,
n=0, 1, 2, or 3;
L is selected form —(CH₂)ₘ—, —(CH₂)ₘ(CR⁵R⁶)—, or —(CH₂)ₘQ-;
m=0, 1, 2, or 3;
X is C, O, or S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl; wherein the substituted C1-C6 alkyl, substituted C3-C6 cycloalkyl, substituted aryl, substituted heteroaryl are substituted by 1-3 substituents independently selected from the following groups: halogen, C1-C3 alkyl, C1-C3 alkoxy, halogenated C1-C3 alkyl, heteroaryl, heterocycle;
alternatively, R¹ and R² together with the nitrogen atom to which they are attached form a substituted or unsubstituted heterocyclic group, with representative structures (but not limited to) as shown below:

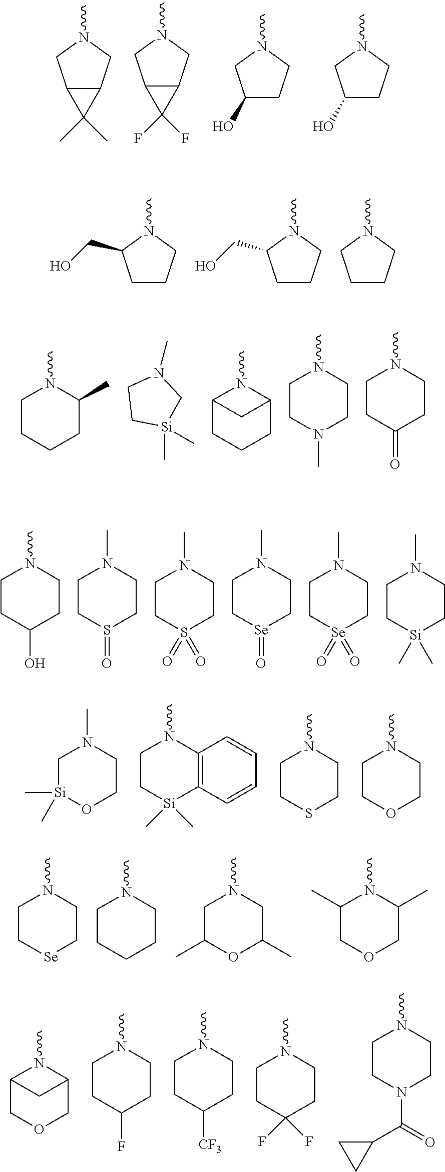

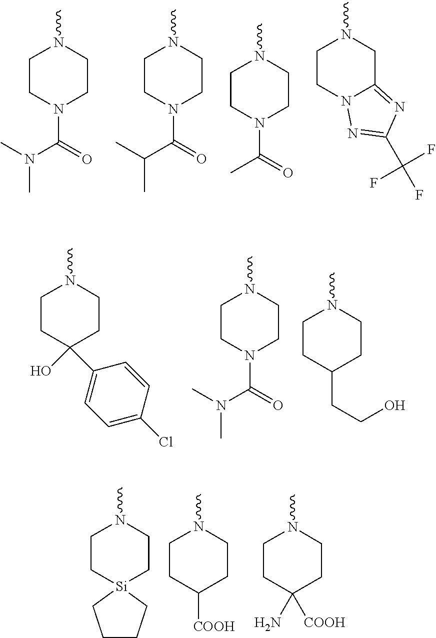

Q is phenyl or heteroaryl groups, wherein phenyl and heteroaryl groups are optionally further substituted by 0-5 R³ groups;
R³ is halogens, C1-C6 alkyl groups, or C3-C6 cycloalkyl groups;
wherein, Y is C5-C14 heteroaryl, and the heteroaryl is optionally further substituted by 0-5 R⁴ groups; the heteroaryl contain 1-4 heteroatoms selected from N, O, or S;
R⁴ is selected from the group consisting of hydrogen, halogens, cyano, C1-C6 alkyl groups, substituted C1-C6 alkyl groups, C3-C6 cycloalkyl groups, substituted C3-C6 cycloalkyl groups, aryl groups, substituted aryl groups, heterocycles, and substituted heterocycles; the substituted C1-C6 alkyl, substituted C3-C6 cycloalkyl, substituted aryl, and substituted heteroaryl are substituted by 1-3 substituents independently selected from the group consisting of halogens, C1-C3 alkyl, C1-C3 alkoxy, halogenated C1-C3 alkyl, heteroaryl, and heterocycle; and
R⁵ and R⁶ are independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

The present disclosure is also directed to a pharmaceutical composition comprising a compound of formula (I), or its stereoisomers, pharmaceutically acceptable salts, solvates, deuterated forms, metabolites, or prodrugs, and a pharmaceutically acceptable carrier or excipient.

The present disclosure is further directed to a method to inhibit a Sigma-1 receptor in a subject. The method includes administering a compound of formula (I) to the subject.

The present disclosure is further directed to a method to treat and/or prevent diseases or disorders related to a Sigma-1 receptor in a subject. The method includes administering a compound of formula (I) to the subject.

Aspects of the present disclosure are related to a method to treat and/or prevent one or more conditions in a subject. The one or more conditions include but are not limited to pain, psychosis, substance abuse, or cancer. The method includes administering a compound of formula (I) to the subject.

### BRIEF DESCRIPTION OF THE FIGURES

The figures are provided to facilitate an understanding of the technical solutions proposed in this application and form part of the specification, used together with the embodiments of this application to explain the technical solutions of this application and do not limit the scope of the technical solutions of this application.

FIG. 1 shows the structures of reference compounds 1-3 involved in this invention.

FIG. 2 illustrates the analgesic effects of the compounds of this invention in the Spinal Nerve Ligation (SNL) model.

### DETAILED DESCRIPTION

This invention provides a new class of Sigma-1 antagonists, along with their preparation methods and applications. The compounds of this invention possess good Sigma-1 antagonistic activity and are capable of providing effective pain management to meet patient needs.

In a second aspect, the invention provides a pharmaceutical composition including the Sigma-1 antagonists and its stereoisomers, pharmaceutically acceptable salts, solvates, deuterated forms, metabolites, or prodrugs, and at least one pharmaceutically acceptable excipient or carrier.

In a third aspect, the invention provides a method for synthesizing the Sigma-1 antagonists.

In a fourth aspect, the invention provides a method for treating diseases or disorders related to Sigma-1. The method involves administering a composition containing compound of formula (I) to an individual in need thereof.

### 1. Sigma-1 Antagonists

In a first aspect, within the embodiments of the present invention, compounds as represented by formula (I), or their stereoisomers, pharmaceutically acceptable salts, solvates, deuterated forms, metabolites, or prodrugs are provided.

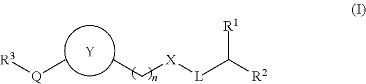

wherein,
n=0, 1, 2, 3;
L is selected from —(CH₂)ₘ—, —(CH₂)ₘ(CR⁵R⁶)—, —(CH₂)ₘQ-;
m=0, 1, 2, 3;
X is selected from C, O, S;
R¹ and R² are each independently selected from the group consisting hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl; the substituted C1-C6 alkyl, substituted C3-C6 cycloalkyl, substituted aryl, substituted heteroaryl are substituted by 1-3 substituents independently selected from the group consisting of the following groups: halogens, C1-C3 alkyl, C1-C3 alkoxy, halogenated C1-C3 alkyl, heteroaryl, and heterocycle;
alternatively, R¹ and R² together with the nitrogen atom to which they are attached may form a substituted or unsubstituted heterocyclic group, with representative (but not limited to) structures as follows:

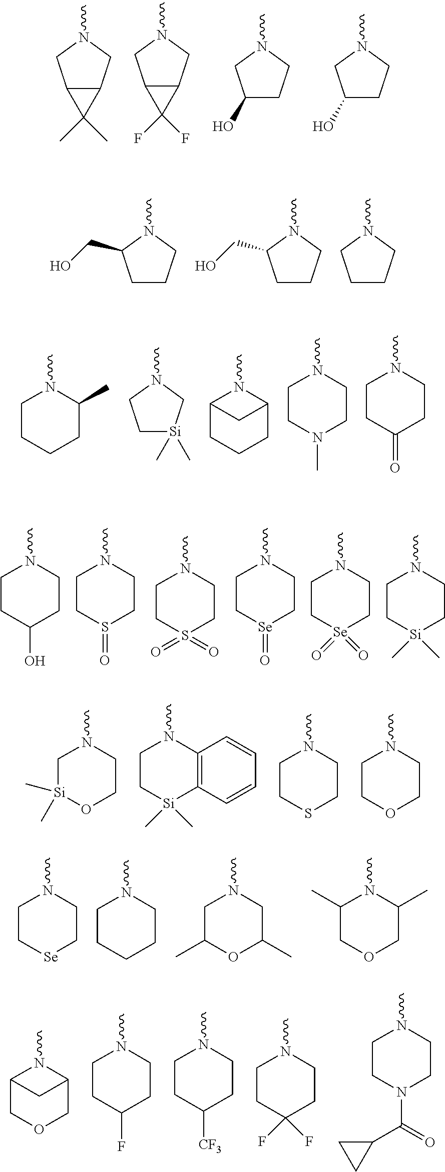

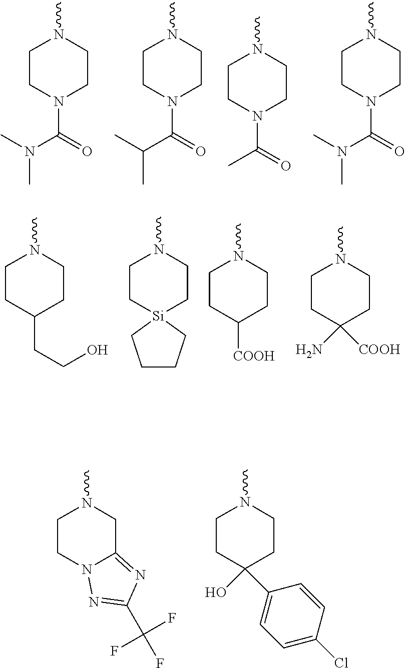

Q is selected from aryl, heteroaryl, and said aryl, heteroaryl are optionally further substituted by 0-5 R³ groups;
R³ is halogen, C1-C6 alkyl, or C3-C6 cycloalkyl;
wherein, Y is selected from C5-C14 heteroaryl, and said heteroaryl is optionally further substituted by 0-5 R⁴ groups; the heteroaryl includes 1-4 heteroatoms selected from N, O, S;
R⁴ is selected from the group consisting of hydrogen, halogens, cyano, C1-C6 alkyl, substituted C1-C6 alkyl, C3-C6 cycloalkyl, substituted C3-C6 cycloalkyl, aryl, substituted aryl, heterocycle, and substituted heterocycle; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

Specifically, in some embodiments, the invention provides a compound as represented by formula (I), and its stereoisomers, pharmaceutically acceptable salts, solvates, deuterated forms, metabolites, or prodrugs;

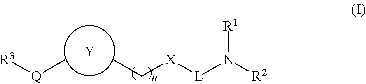

wherein,
n=0, 1, 2, or 3;
L is selected from —(CH₂)ₘ—, —(CH₂)ₘ(CR⁵R⁶)—, or —(CH₂)ₘQ-;
m=0, 1, 2, or 3;
X is selected from C, O, or S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle;
alternatively, R¹ and R² together with the nitrogen atom to which they are attached may form a substituted or unsubstituted heterocyclic group, with representative (but not limited to) structures as follows:

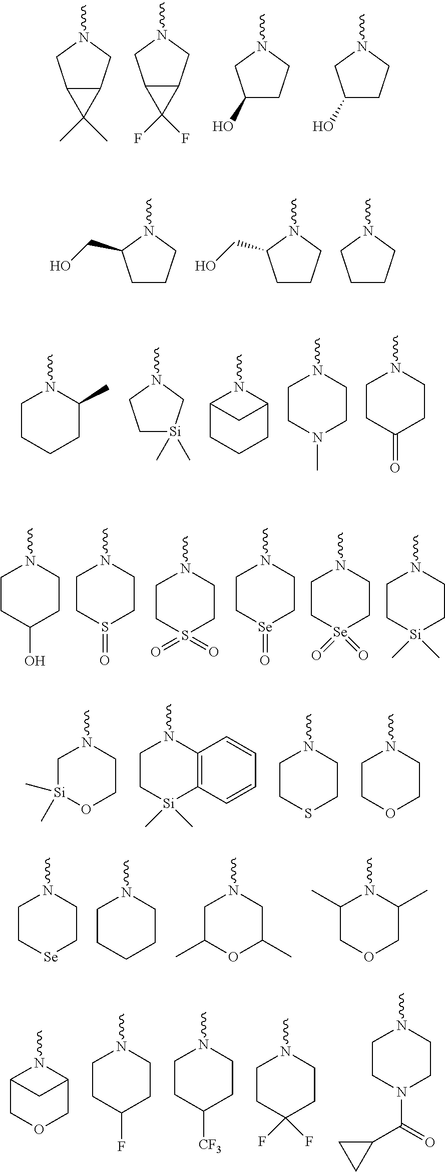

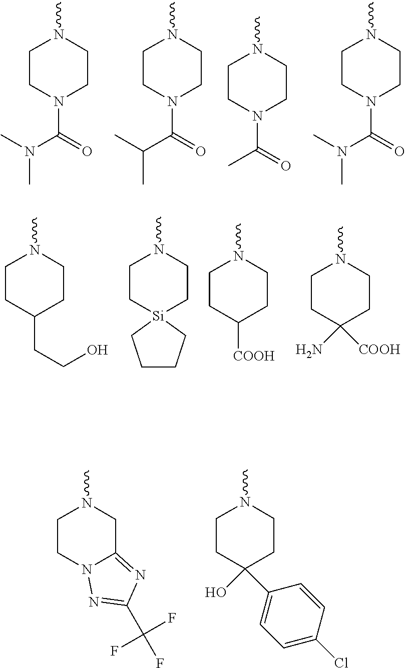

Q is aryl or heteroaryl, and the aryl, heteroaryl are optionally further substituted by 0-5 R³ groups;
R³ is halogens, C1-C6 alkyl, or C3-C6 cycloalkyl;
wherein, Y is C5-C14 heteroaryl, and the heteroaryl is optionally further substituted by 0-5 R⁴ groups; the heteroaryl includes 1-4 heteroatoms selected from N, O, S;
R⁴ is selected from the group consisting of hydrogen, halogens, cyano, C1-C6 alkyl, substituted C1-C6 alkyl, C3-C6 cycloalkyl, substituted C3-C6 cycloalkyl, aryl, substituted aryl, heterocycle, and substituted heterocycle; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

Specifically, in some embodiments, this disclosure provides a compound as represented by formula (II), or its stereoisomers, pharmaceutically acceptable salts, solvates, deuterated forms, metabolites, or prodrugs.

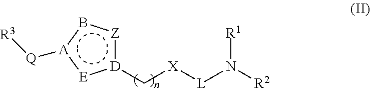

wherein,
n=0, 1, 2, or 3;
L is selected from —(CH₂)ₘ—, —(CH₂)ₘ(CR⁵R⁶)—, or —(CH₂)ₘQ-;
m=0, 1, 2, or 3;
X is selected from C, O, or S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl; the substituted C1-C6 alkyl, substituted C3-C6 cycloalkyl, substituted aryl, substituted heteroaryl are substituted by 1-3 substituents independently selected from the group consisting of halogens, C1-C3 alkyl, C1-C3 alkoxy, halogenated C1-C3 alkyl, heteroaryl, and heterocycle;
alternatively, R¹ and R² together with the nitrogen atom to which they are attached may form a substituted or unsubstituted heterocyclic group, as shown in above;
Q is aryl, heteroaryl, or the aryl, wherein heteroaryl is optionally further substituted by 0-5 R³ groups; R³ is halogens, C1-C6 alkyl, or C3-C6 cycloalkyl; A, B, D, E, Z are each independently C, N, or O;
wherein, when any one of B, Z, E is selected from C, it can be connected to R⁴;
R⁴ is selected from the group consisting of hydrogen, halogens, cyano, C1-C6 alkyl, substituted C1-C6 alkyl, C3-C6 cycloalkyl, substituted C3-C6 cycloalkyl, aryl, substituted aryl, heterocycle, and substituted heterocycle; the substituted C1-C6 alkyl, substituted C3-C6 cycloalkyl, substituted aryl, substituted heteroaryl are substituted by 1-3 substituents independently selected from the group consisting of halogens, C1-C3 alkyl, C1-C3 alkoxy, halogenated C1-C3 alkyl, heteroaryl, and heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

In some specific embodiments, when A, D, E are carbon atoms, B is a nitrogen atom, and Z is an oxygen atom, as shown in formula (IIa);

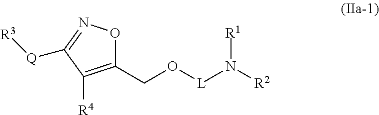

wherein, when X is an oxygen atom, n=1, as shown in formula (IIa-1):
wherein, L is —(CH₂)ₘ—, —(CH₂)ₘ(CR⁵R⁶)—, or —(CH₂)ₘQ-, as shown in formulas (IIa-1a), (IIa-1b), and (IIa-1c):
when X is a carbon atom, n=0, and L is —(CH₂)ₘ—, with m=0, as shown in formula (IIa-2):

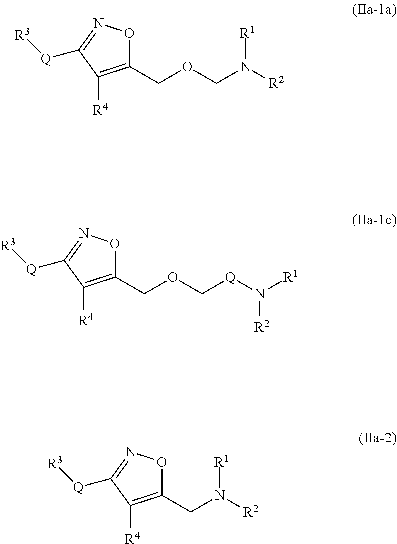

R¹ and R² are each independently selected from the group consisting of hydrogen, methyl, ethyl, propyl, isopropyl, butyl, cyclopropyl, cyclobutyl, cyclopentyl, cyclohexyl, phenyl, naphthyl, and pyridine; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine;
alternatively, R¹ and R² together with the nitrogen atom to which they are attached may form a substituted or unsubstituted heterocyclic group, as shown above;
Q is selected from the group consisting of phenyl, naphthyl, pyrrolyl, pyrazolyl, pyridyl, quinolyl, and pyrazinyl, and optionally further substituted by 0-5 R³ groups;
R³ is selected from fluorine, chlorine, bromine, methyl, ethyl, propyl, isopropyl, butyl, tert-butyl, cyclopropyl, cyclobutyl, cyclopentyl, cyclohexyl;
R⁴ is selected from the group consisting of hydrogen, fluorine, chlorine, bromine, cyano, methyl, ethyl, propyl, isopropyl, butyl, tert-butyl, cyclopropyl, cyclobutyl, cyclopentyl, cyclohexyl, phenyl, naphthyl, and pyridine; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine; and
R⁵ and R⁶ are each independently selected from the group consisting of hydrogen, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

In some embodiments, R¹ and R² are each independently selected from the group consisting of hydrogen, methyl, ethyl, propyl, isopropyl, butyl, and tert-butyl; or R¹ and R² together with the nitrogen atom to which they are attached may form a substituted or unsubstituted heterocyclic group, selected from groups as shown below:

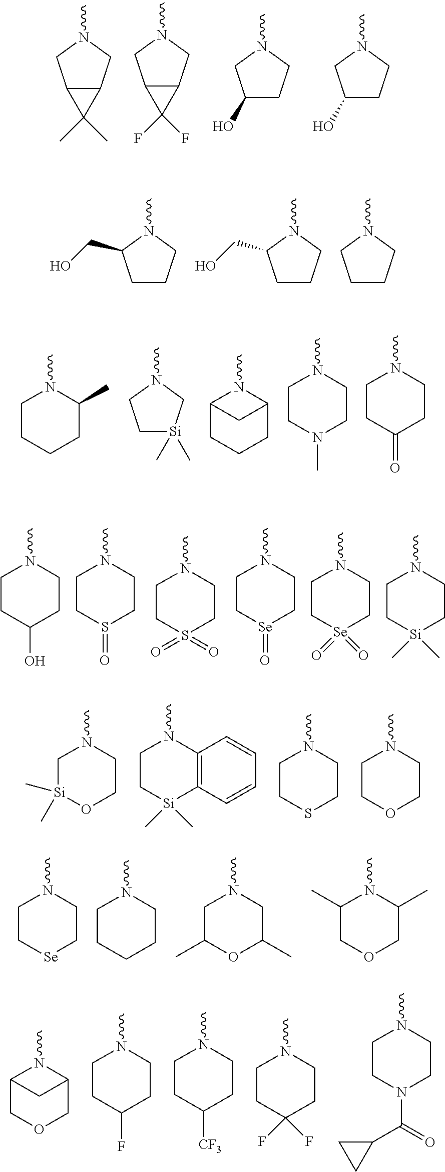

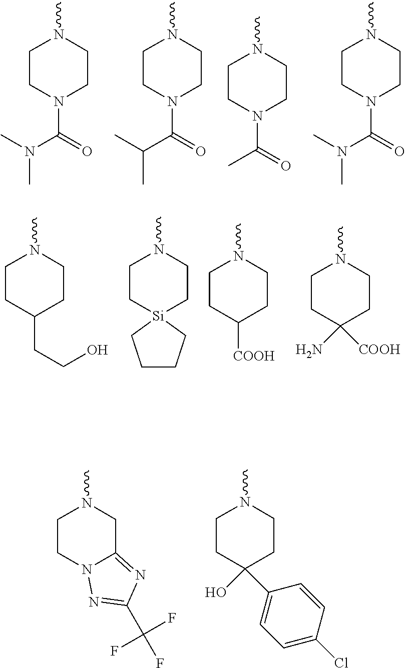

In some embodiments, Q is phenyl, naphthyl, or pyridyl, and is further substituted by 0-5 R³ groups.

In some embodiments, R³ is selected from the group consisting of fluorine, chlorine, bromine, methyl, ethyl, propyl, isopropyl, butyl, and tert-butyl.

In some embodiments, R⁴ is hydrogen, fluorine, chlorine, bromine, or thiophenyl.

In some embodiments, R⁵ and R⁶ are each independently hydrogen or methyl.

In some specific embodiments, when B, Z, D are carbon atoms, and A and E are nitrogen atoms, as shown in formula (IIb):

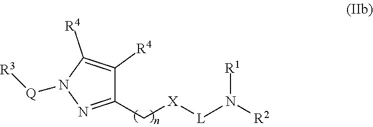

L is —(CH₂)ₘ—, —(CH₂)ₘ(CR⁵R⁶)—, or —(CH₂)ₘQ-;

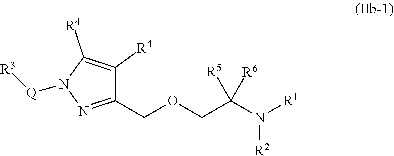

wherein, when X is O, n=2, L is —(CH₂)ₘ(CR⁵R⁶)—, and m=2, as shown in formula (IIb-1):
wherein, when X is —CH₂—, n=0, and L is —(CH₂)ₘ—, with m=0, as shown in formula (IIb-2):

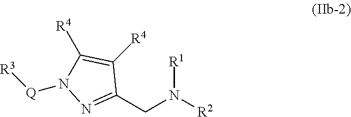

R¹ and R² are each independently selected from the group consisting of hydrogen, methyl, ethyl, propyl, isopropyl, butyl, cyclopropyl, cyclobutyl, cyclopentyl, cyclohexyl, phenyl, naphthyl, and pyridine; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine;
alternatively, R¹ and R² together with the nitrogen atom to which they are attached may form a substituted or unsubstituted heterocyclic group;
Q is phenyl, naphthyl, pyrrolyl, pyrazolyl, pyridyl, quinolyl, or pyrazinyl, and optionally further substituted by 0-5 R³ groups;
R³ is selected from the group consisting of fluorine, chlorine, bromine, methyl, ethyl, propyl, isopropyl, butyl, tert-butyl, cyclopropyl, cyclobutyl, cyclopentyl, and cyclohexyl;
R⁴ is selected from the group consisting of hydrogen, fluorine, chlorine, bromine, cyano, methyl, ethyl, propyl, isopropyl, butyl, tert-butyl, cyclopropyl, cyclobutyl, cyclopentyl, cyclohexyl, phenyl, naphthyl, and pyridine; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine; and
R⁵ and R⁶ are each independently selected from the group consisting of hydrogen, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

In some embodiments, R¹ and R² together with the nitrogen atom to which they are attached may form a substituted or unsubstituted heterocyclic group, selected from group consisting of

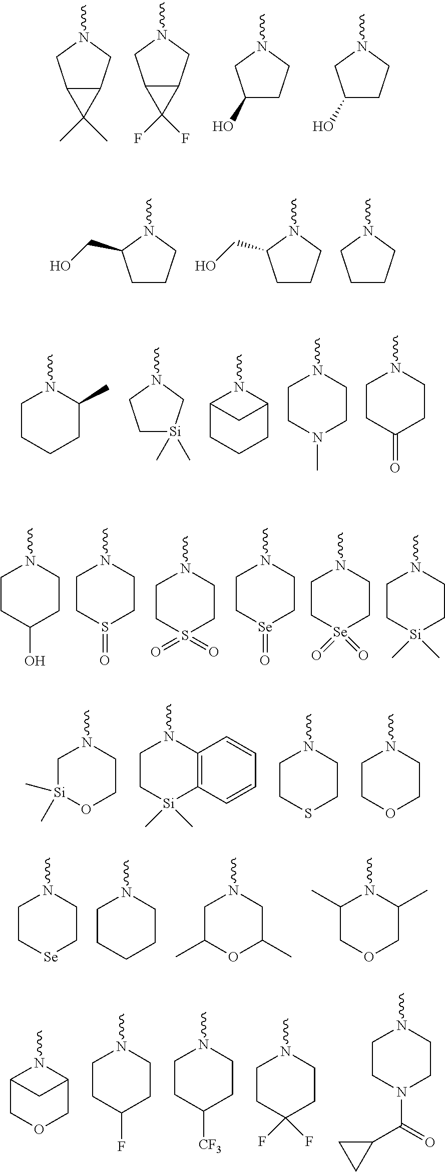

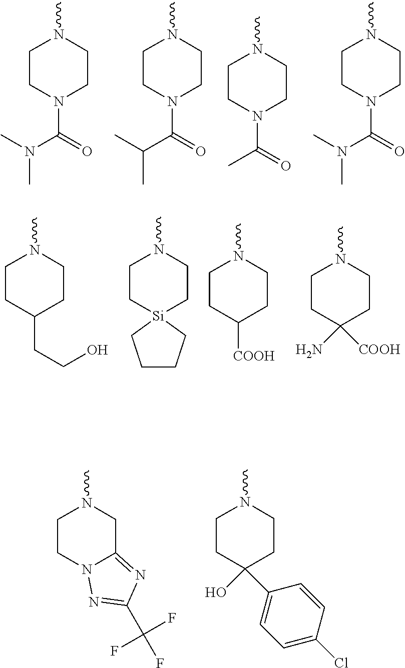

In some embodiments, Q is phenyl or naphthyl, wherein Q is further substituted by 0-5 R³ groups.

In some embodiments, R³ is fluorine, chlorine, or bromine.

In some embodiments, R⁴ is hydrogen;

In some embodiments, R⁵ and R⁶ are hydrogen.

Specifically, a compound as represented by formula (III), or its stereoisomers, pharmaceutically acceptable salts, solvates, deuterated forms, metabolites, or prodrugs;

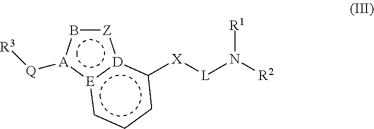

wherein,
L is selected from —(CH₂)ₘ—, —(CH₂)ₘ(CR⁵R⁶)—, —(CH₂)ₘQ-;
m=0, 1, or 2;
X is C, O, or S;
R¹ and R² are each independently selected from the group consisting hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl are substituted by 1-3 substituents independently selected from the group consisting of halogens, C1-C3 alkyl, C1-C3 alkoxy, halogenated C1-C3 alkyl, heteroaryl, and heterocycle;
alternatively, R¹ and R² together with the nitrogen atom to which they are attached may form a substituted or unsubstituted heterocyclic group, as shown in above;
Q is aryl or heteroaryl, and the aryl, heteroaryl are optionally further substituted by 0-5 R³ groups;
R³ is halogens, C1-C6 alkyl, C3-C6 cycloalkyl;
A, B, D, E, Z are each independently C, N, or O;
wherein, when any one of B, Z, E is selected from C, it can be connected to R⁴;
R⁴ is selected from the group consisting of hydrogen, halogens, cyano, C1-C6 alkyl, substituted C1-C6 alkyl, C3-C6 cycloalkyl, and substituted C3-C6 cycloalkyl; the substituted C1-C6 alkyl, substituted C3-C6 cycloalkyl are substituted by 1-3 substituents independently selected from the group consisting of halogens, C1-C3 alkyl, C1-C3 alkoxy, halogenated C1-C3 alkyl, heteroaryl, and heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

In some specific embodiments, when Z, D, E are carbon atoms and A and B are nitrogen atoms, as shown in formula (IIIa):

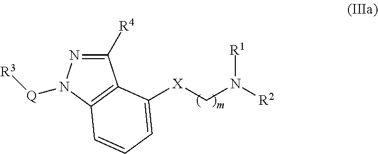

In some specific embodiments, when A and D are carbon atoms, and B, Z, E are nitrogen atoms, as shown in formula (IIIb):

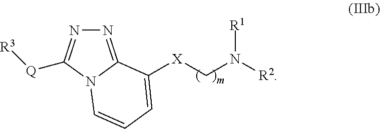

In some specific embodiments, when A, D, E are carbon atoms, B is a nitrogen atom, and Z is an oxygen atom, as shown in formula (IIIc):

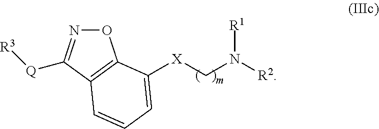

In some specific embodiments, when A, Z, D are carbon atoms, and B and E are nitrogen atoms, as shown in formula (IIId):

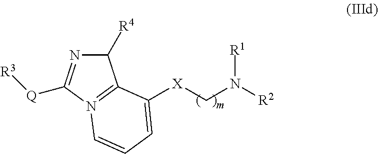

wherein,
m=0, 1, 2, or 3;
X is C, O, or S;
R¹ and R² are each independently selected from hydrogen, methyl, ethyl, propyl, isopropyl, butyl, cyclopropyl, cyclobutyl, cyclopentyl, cyclohexyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine;
alternatively, R¹ and R² together with the nitrogen atom to which they are attached may form a substituted or unsubstituted heterocyclic group;
Q is selected from phenyl, naphthyl, pyrrolyl, pyrazolyl, pyridyl, quinolyl, pyrazinyl, and optionally further substituted by 0-5 R³ groups;
R³ is selected from fluorine, chlorine, bromine, methyl, ethyl, propyl, isopropyl, butyl, tert-butyl, cyclopropyl, cyclobutyl, cyclopentyl, cyclohexyl; and
R⁴ is selected from hydrogen, fluorine, chlorine, bromine, cyano, methyl, ethyl, propyl, isopropyl, butyl, tert-butyl, cyclopropyl, cyclobutyl, cyclopentyl, cyclohexyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine.

In some embodiments, m=0, 1, or 2.

In some embodiments, X is a carbon atom or an oxygen atom.

In some embodiments, R¹ and R² together with the nitrogen atom to which they are attached may form a substituted or unsubstituted heterocyclic group, selected from groups as shown below:

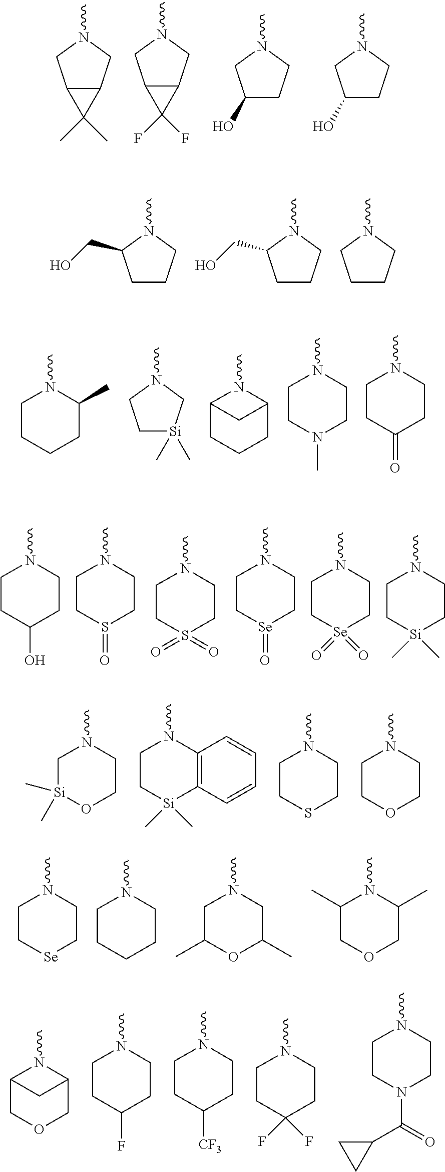

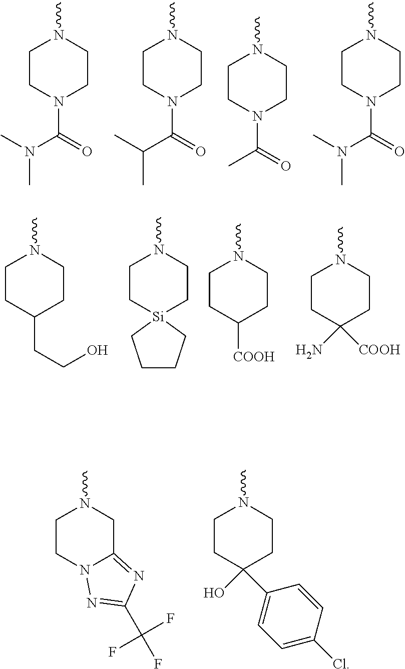

In some embodiments, Q is phenyl or pyridyl, and is further substituted by 0-5 R³ groups.

In some embodiments, R³ is fluorine, chlorine, or bromine;

In some embodiments, R⁴ is hydrogen, fluorine, chlorine, or bromine;

Furthermore, the compounds of formula (I) are selected from the following compounds, and their stereoisomers, pharmaceutically acceptable salts, solvates, deuterated forms, metabolites, or prodrugs:

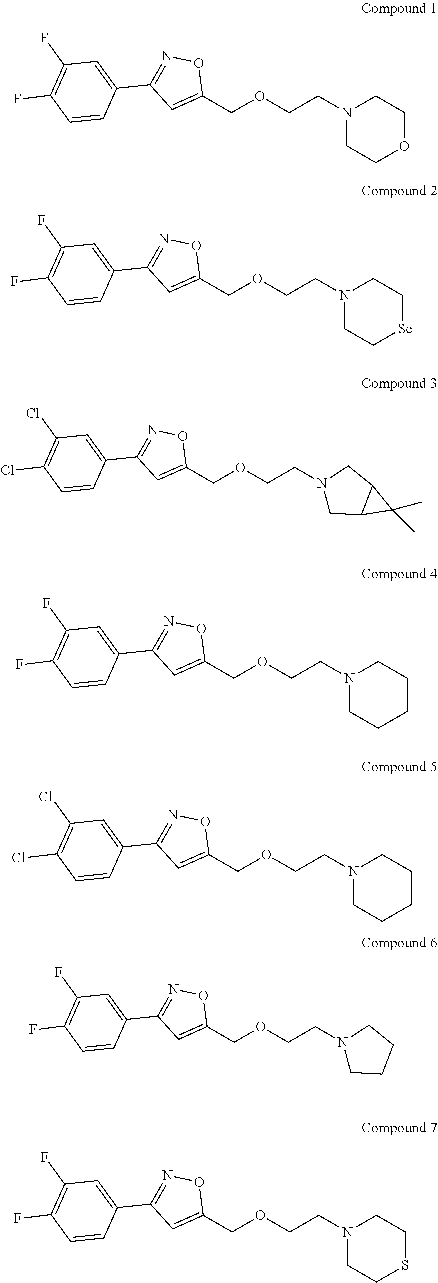

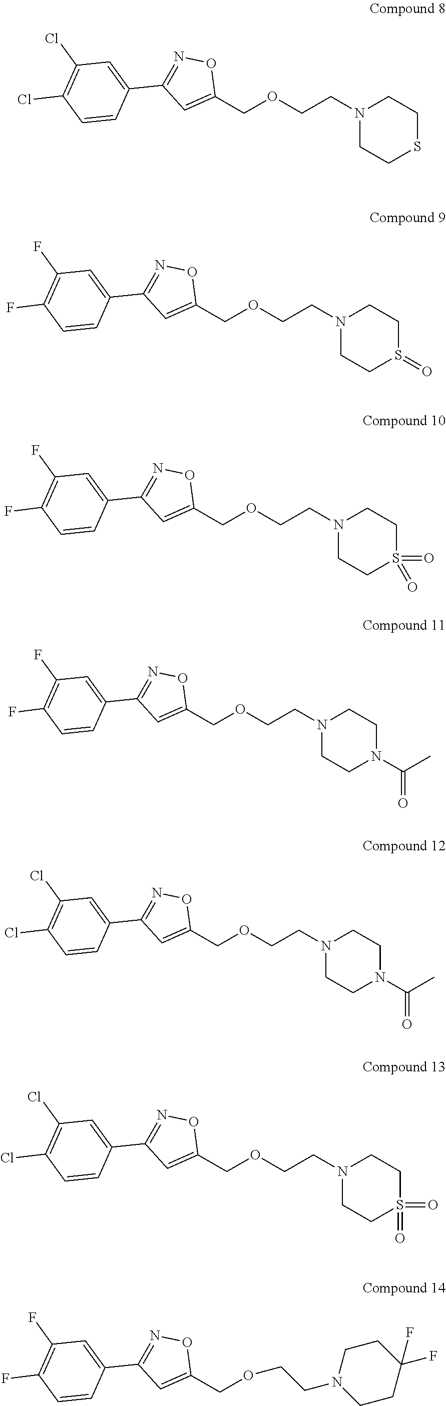

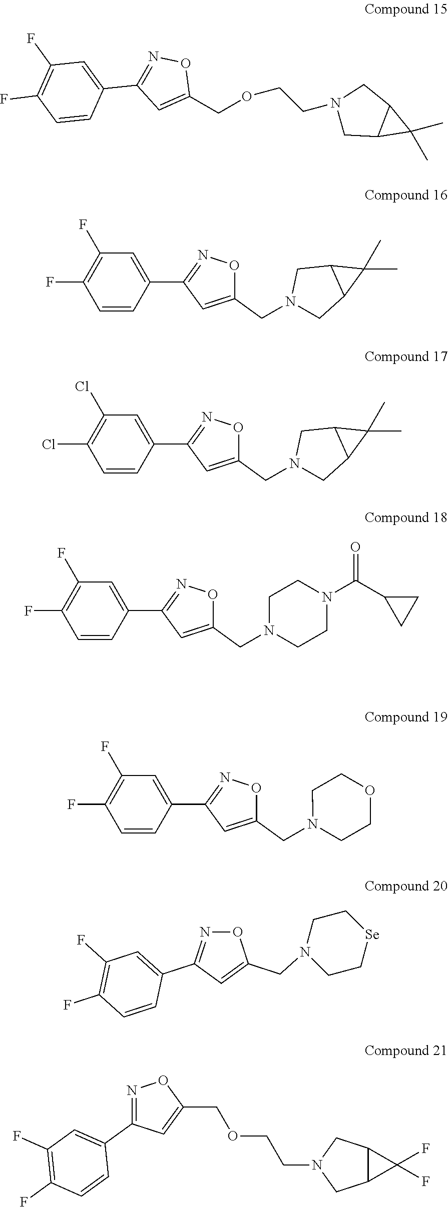

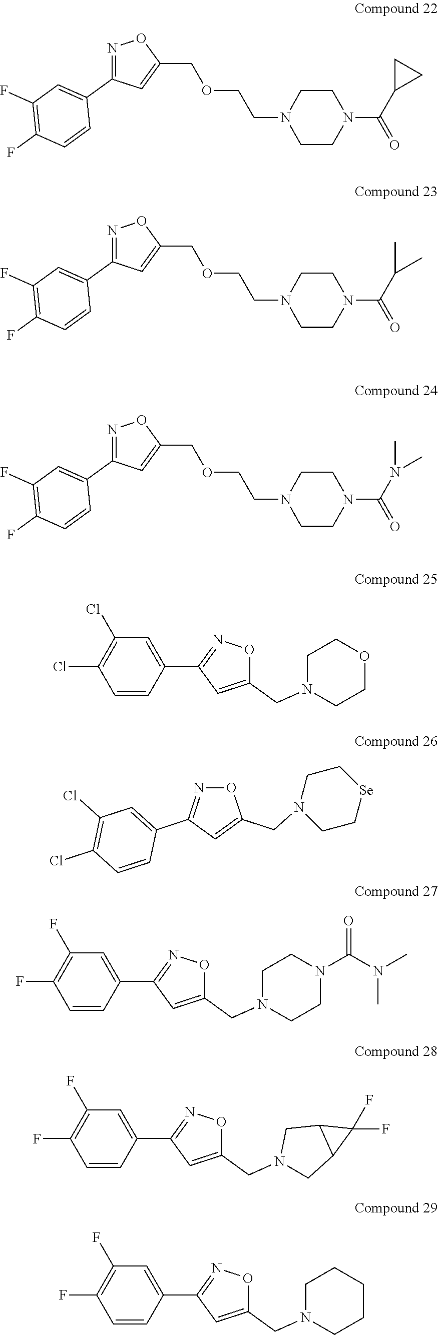

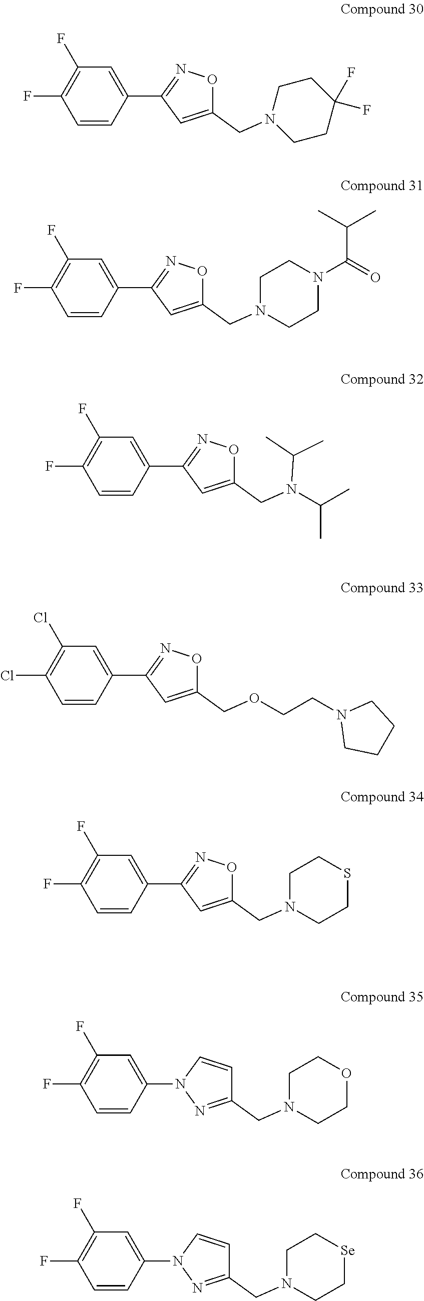

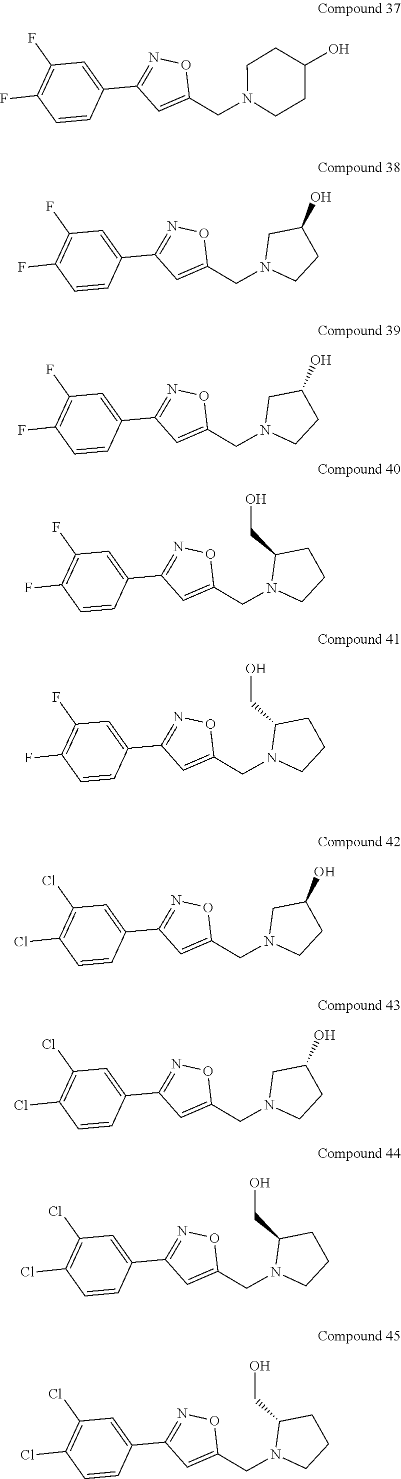

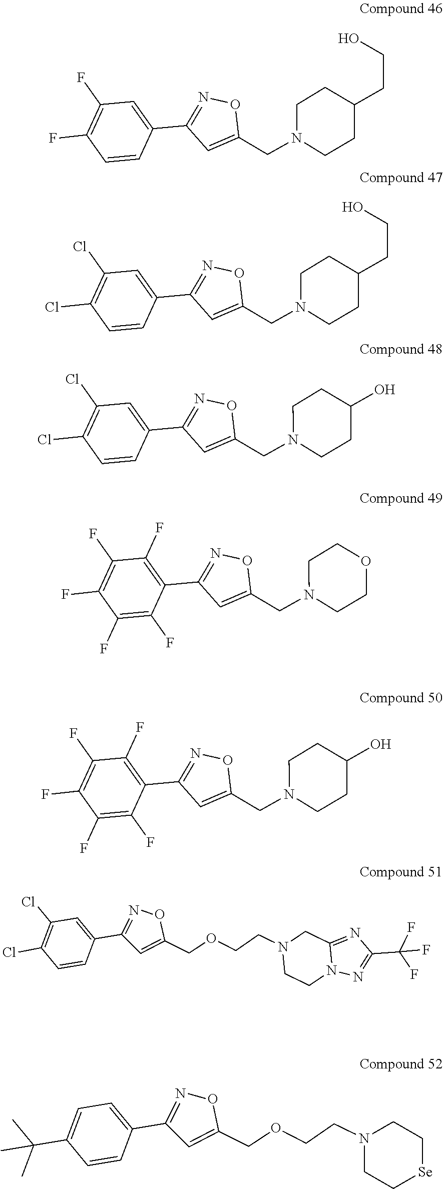

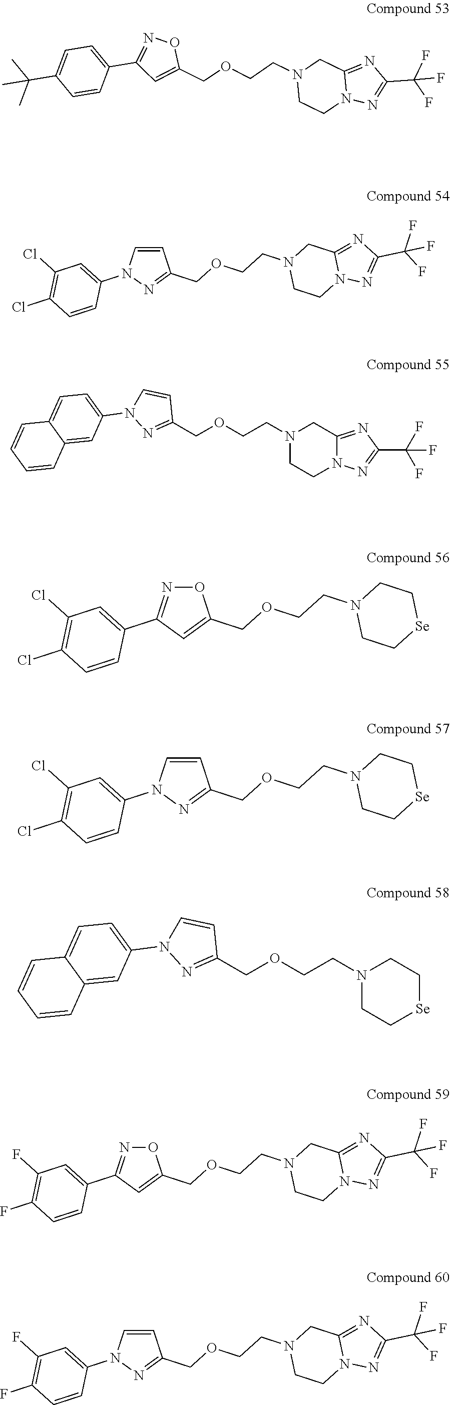

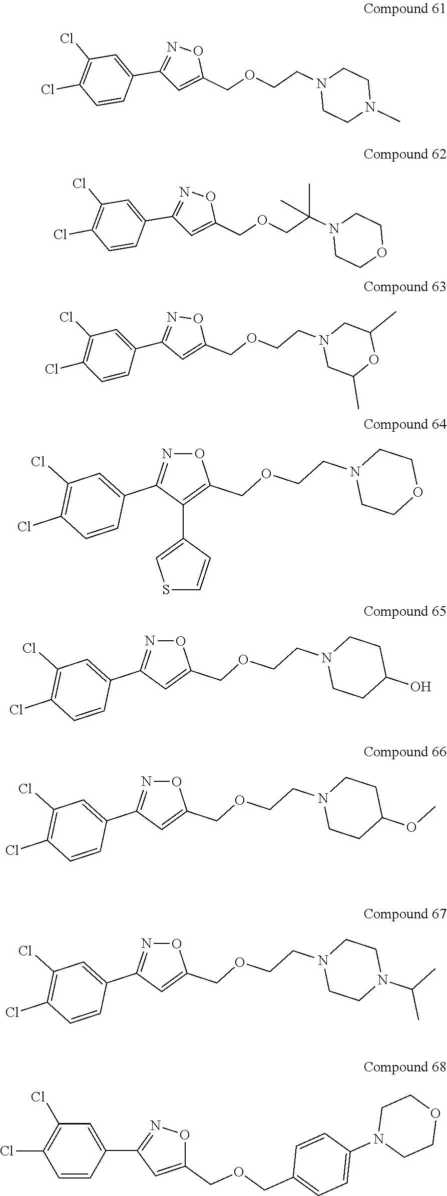

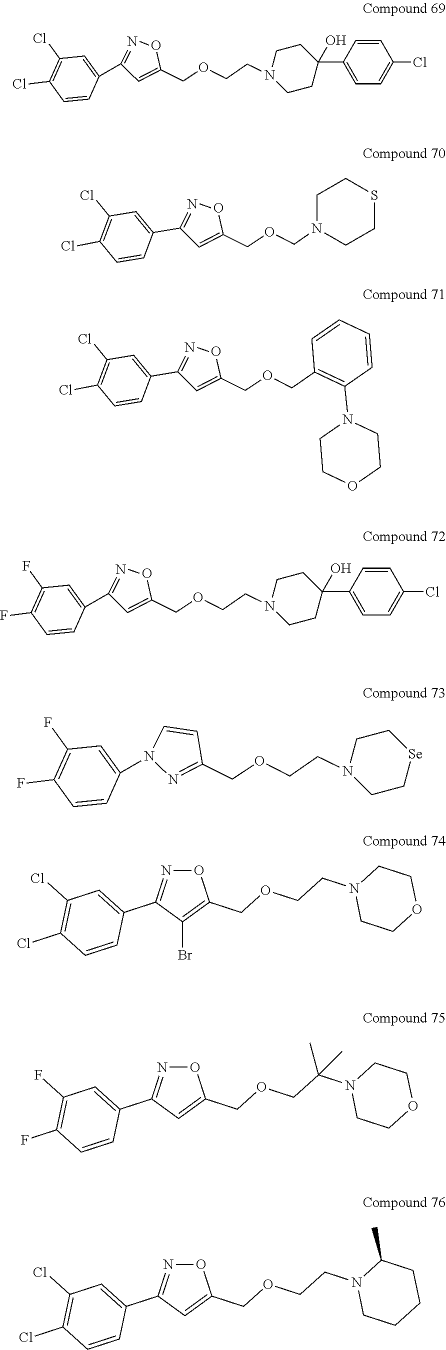

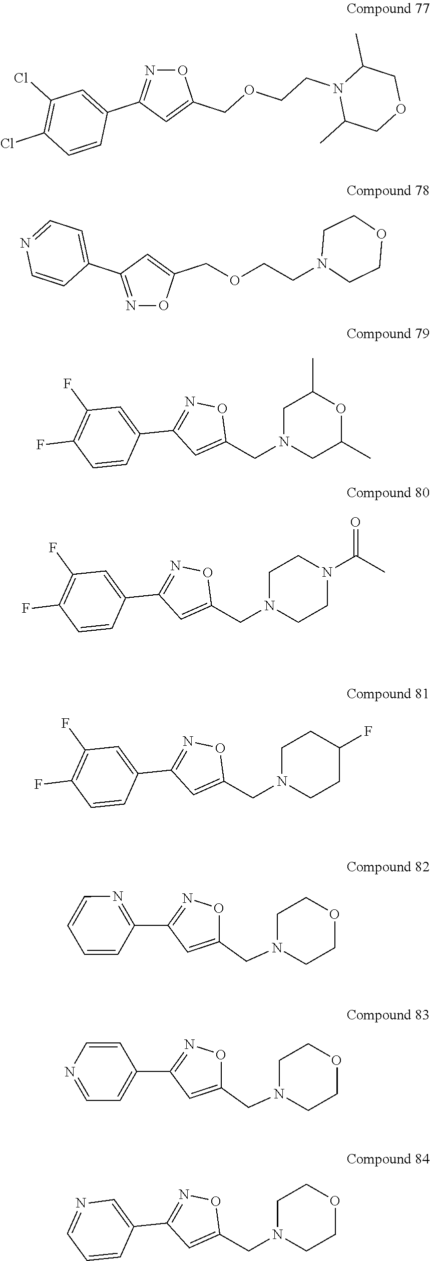

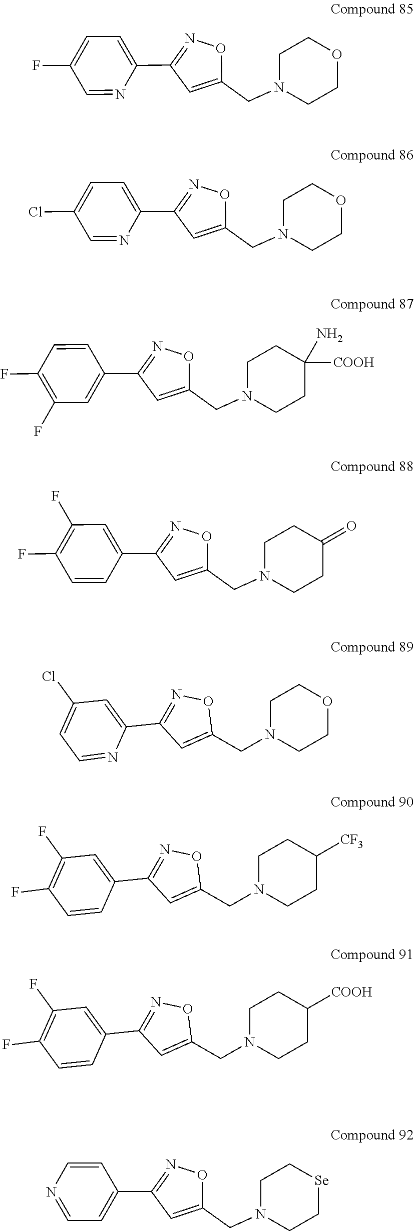

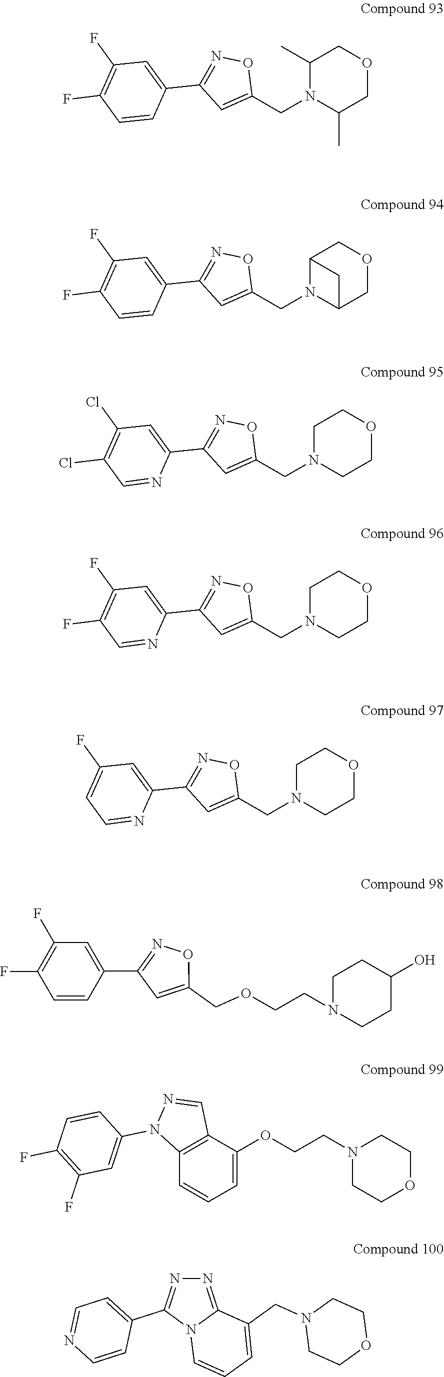

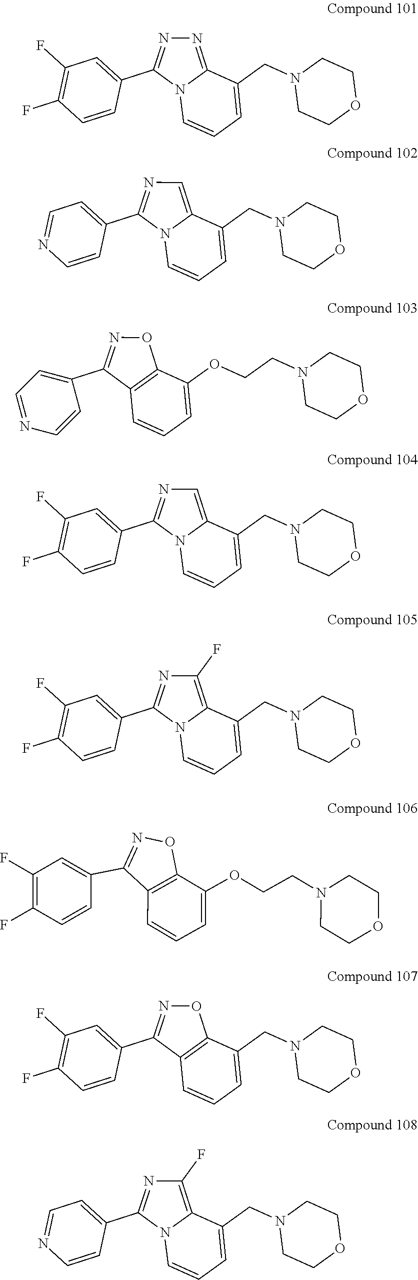

### II. A Pharmaceutical Composition

In some embodiments, the invention provides a pharmaceutical composition. The pharmaceutical composition includes a compound of formula (I) and its stereoisomers, pharmaceutically acceptable salts, solvates, deuterated forms, metabolites, or prodrugs, and at least one pharmaceutically acceptable excipient.

The compound is described above in Section I.

The disclosed pharmaceutical composition contains at least one pharmaceutically acceptable excipient. Non-limiting examples of suitable excipients may include diluents, binders, fillers, buffers, pH adjusters, disintegrants, dispersants, stabilizers, preservatives, and colorants. The amount and type of excipients can be selected based on known pharmaceutical principles.

The pharmaceutical composition can be mixed with one or more excipients to form solid, liquid, or semisolid dosage forms. The methods of formulating solid, liquid, or semisolid dosage forms are known in the art.

### III. Method of Making Sigma-1 Antagonists

In some embodiments, this application provides a method for preparing the above compounds or their stereoisomers, pharmaceutically acceptable salts, solvates, deuterated forms, metabolites, or prodrugs, which includes the following steps:

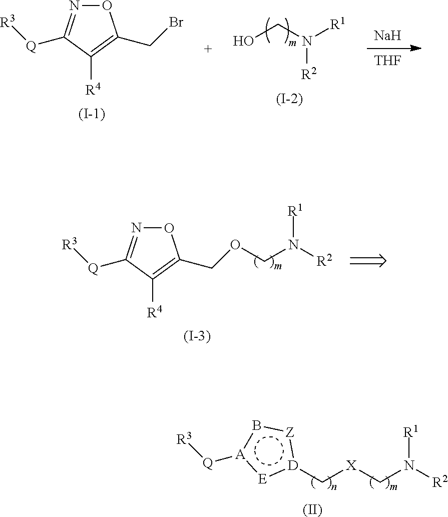

Compound of formula (I-1) may react with compound of formula (I-2) under the conditions of sodium hydride and tetrahydrofuran in a nucleophilic substitution reaction to obtain compound of formula (I-3), i.e., compound of formula (II);

Alternatively, compound of formula (I-1) may react with compound of formula (I-4) under the conditions of potassium carbonate/cesium carbonate in acetonitrile in a nucleophilic substitution reaction to obtain compound of formula (I-5), i.e., compound of formula (II);

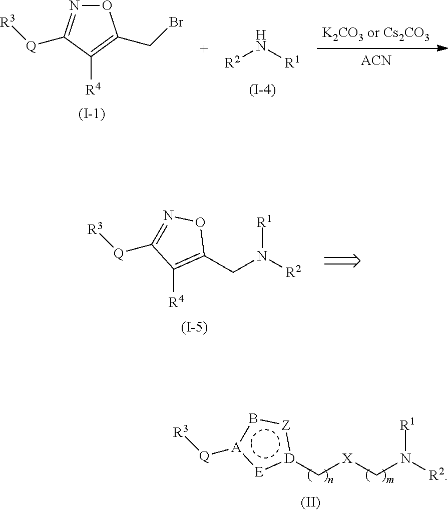

Alternatively, compound of formula (I-6) may react with compound of formula (I-7) under the conditions of sodium fluoride in dichloromethane in a cyclization reaction to obtain compound of formula (I-8), i.e., compound of formula (II);

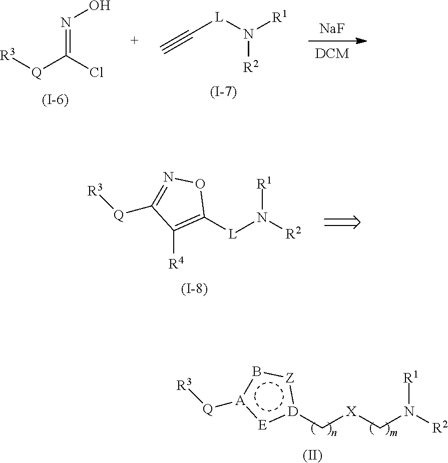

Alternatively, compound of formula (I-1) may react with compound of formula (I-9) under the conditions of potassium tert-butoxide in tetrahydrofuran to obtain compound of formula (I-10), which is then subjected to a heated reaction in a system of acetic acid/tetrahydrofuran/water to yield compound of formula (I-11);

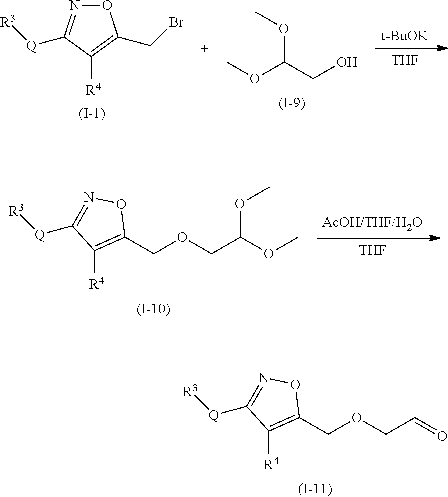

Compound of formula (I-11) may react with compound of formula (I-4) under the conditions of sodium triacetoxyborohydride in tetrahydrofuran/dichloromethane in a reductive amination reaction to obtain compound of formula (I-12), i.e., compound of formula (II);

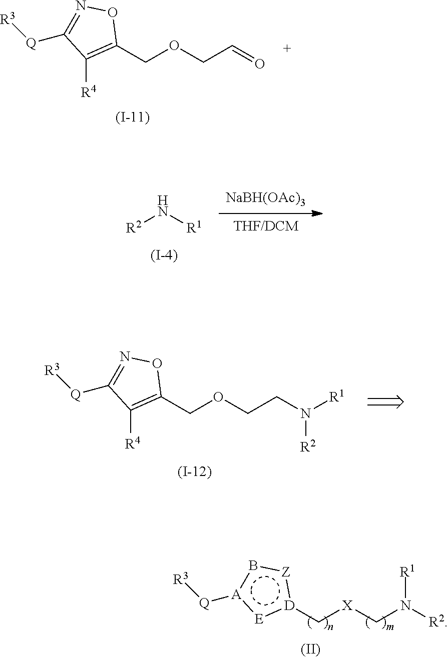

### IV. Method of Use

On another aspect, the invention provides a method for treating diseases or disorders related to the Sigma-1 receptor, wherein the method includes administering a pharmaceutical composition to a subject in need thereof, the pharmaceutical composition comprising a compound of formula (I).

Without being bound by any theory, compounds of formula (I) are believed to act primarily as antagonists mediating the activity of the Sigma-1 receptor. Binding at this site is thought to be therapeutic for conditions related to pain, among others.

These compounds can be administered through various routes. For example, compounds of formula (I) can be administered orally in solid or liquid dosage forms (tablets, gel caps, controlled-release capsules, powders, solutions, or suspensions in aqueous or non-aqueous liquids), parenterally (i.e., subcutaneous, intradermal, intravenous (i.e., as a solution, suspension, or emulsion in a carrier), intramuscular, intracranial, or intraperitoneal injections), or topically (i.e., transdermal or transmucosal administration, including but not limited to oral, rectal, vaginal, and sublingual).

In one embodiment, the compound can be administered in saline or together with the aforementioned pharmaceutically acceptable excipients. The compound can be used as a primary therapy or as an adjunctive therapy, administered following local interventions (surgery, radiation, local chemotherapy), or in combination with at least one other chemotherapy agent.

Suitable subjects may include but are not limited to humans and companion animals such as cats, dogs, rodents, and horses; research animals such as rabbits, sheep, pigs, dogs, primates, mice, rats, and other rodents; livestock such as cattle, cows, pigs, goats, sheep, horses, deer, chickens, and other poultry; zoo animals; and primates such as chimpanzees, monkeys, and gorillas. The age of the subjects is not limited. In a preferred embodiment, the subject can be human.

Generally, the compound of formula (I) will be administered in a therapeutically effective amount, which includes prophylactic amounts or lower dosages, for example, when used in combination with another formulation. The term “effective amount” as used herein refers to the dosage of the compound sufficient to provide a high enough circulating concentration to have a beneficial effect on the receptor. A skilled physician can determine the precise dosage based on the required dosage, side effects, and the patient's history.

Generally, compounds of formula (I) have an EC50 for binding affinity to the Sigma-1 receptor less than about 100 nM. In various embodiments, the EC50 of the compounds composed of formula (I) is less than about 100 nM, or less than 10 nM, or less than about 5 nM, or less than about 1 nM.

### Beneficial Effects

The compounds of this invention are capable of binding to the Sigma-1 receptor and possess good Sigma-1 receptor antagonistic activity, making them suitable for medicinal use with clinical application value. Additionally, the synthesis steps of the compounds in this application are simple, thereby providing good economic value.

### Term Definitions and Descriptions

Unless otherwise specified, the definitions of groups and terms used in this application, including those listed as examples, exemplary, preferred, in tables, or as specific compounds in embodiments, can be combined and interchanged among each other. Such subsequent group definitions and compound structures should be considered within the scope described in the specification.

The compounds described herein may possess asymmetric centers. The compounds of this invention containing asymmetric substituent atoms can be isolated in optically active or racemic forms. Unless a specific stereochemistry or isomeric form is explicitly mentioned, all chiral, achiral, racemic forms, and all geometric isomers of structures are applicable.

As used herein, the phrases “sigma-1 receptor” and “σ1 receptor” are interchangeable and used interchangeably.

The term “alkyl” used herein refers to lower alkyl groups having 1 to 6 carbon atoms in the main chain, and up to 20 carbon atoms in total. They can be straight-chain, branched-chain, or cyclic, including methyl, ethyl, propyl, isopropyl, butyl, hexyl, etc.

The term “aryl” used alone or as part of another group in this text denotes any optionally substituted conjugated planar ring or ring system containing delocalized electrons. These aryl groups are preferably monocyclic ring (e.g., furan or benzene), bicyclic ring, or tricyclic ring groups containing 5-14 atoms in the ring portion. The term “aromatic” includes the defined “aryl”.

The term “aryl” or “Ar” used alone or as part of another group in this text denotes any optionally substituted aromatic group, preferably a single or bi-ring group having 6 to 10 carbon atoms in the ring portion, such as phenyl, biphenyl, naphthyl, substituted phenyl, substituted biphenyl, or substituted naphthyl.

The term “carbocycle” or “carbocyclic” used alone or as part of another group denotes any optionally substituted, aromatic or non-aromatic, monocyclic or polycyclic group where all the atoms in the ring are carbon, preferably with 5 or 6 carbon atoms in each ring. Exemplary substituents include one or more of the following groups: alkyl, substituted alkyl, alkoxy, acyl, acyloxy, alkene, alkenoxy, aryl, aryloxy, amino, amido, acetal, aminocarbonyl, carbocyclic, cyano, ester, ether, halogen, heterocycle, hydroxyl, ketone, ketal, phosphate, nitro, and thio.

The term “heteroaryl” used alone or as part of another group denotes any optionally substituted aromatic group having at least one heteroatom in at least one ring, preferably with 5 or 6 atoms in each ring. Heteroaryl groups preferably have 1 or 2 oxygen atoms and/or 1 to 4 nitrogen atoms in the ring and are connected to the rest of the molecule through carbon. Exemplary groups include furanyl, benzofuranyl, oxazolyl, isoxazolyl, oxadiazolyl, benzoxazolyl, benzoxadiazolyl, pyrrolyl, pyrazolyl, imidazolyl, triazolyl, tetrazolyl, pyridyl, quinolyl, pyrazinyl, quinoxalinyl, indolyl, isoindolyl, indolizinyl, benzimidazolyl, indazolyl, benzotriazolyl, tetrazolopyrazinyl, carbazolyl, purinyl, quinolinyl, isoquinolinyl, imidazopyridinyl, etc. Exemplary substituents include one or more of the following groups: alkyl, substituted alkyl, alkoxy, acyl, acyloxy, alkene, alkenoxy, aryl, aryloxy, amino, amido, acetal, aminocarbonyl, carbocyclic, cyano, ester, ether, halogen, heterocycle, hydroxyl, ketone, ketal, phosphate, nitro, and thio.

The term “heterocycle” or “heterocyclic” used alone or as part of another group denotes any optionally substituted, fully saturated or unsaturated, monocyclic or bicyclic, aromatic or non-aromatic group having at least one heteroatom in at least one ring, preferably with 5 or 6 atoms in each ring. Heterocyclic groups preferably have 1 or 2 oxygen atoms and/or 1 to 4 nitrogen atoms in the ring and are connected to the rest of the molecule through carbon or a heteroatom. Exemplary heterocyclic groups include the heteroaryl compounds described above. Exemplary substituents include one or more of the following groups: alkyl, substituted alkyl, alkoxy, acyl, acyloxy, alkene, alkenoxy, aryl, aryloxy, amino, amido, acetal, aminocarbonyl, carbocyclic, cyano, ester, ether, halogen, heterocycle, hydroxyl, ketone, ketal, phosphate, nitro, and thio.

The term “protecting group” used in this text denotes a group that can protect a specific part of a molecule, where the protecting group can be removed after the protected reaction without interfering with the rest of the molecule. When the part is an oxygen atom (forming a protected hydroxyl group), exemplary protecting groups include ethers (e.g., allyl, triphenylmethyl (tributyl or Tr), benzyl, p-methoxybenzyl (PMB), p-methylphenyl (PMP)), acetals (e.g., methoxymethyl (MOM), β-methoxyethoxymethyl (MEM), tetrahydropyranyl (THP), ethoxyethyl (EE), methylthiomethyl (MTM), 2-methoxy-2-propyl (MOP), 2-trimethylsilylethoxymethyl (SEM)), esters (e.g., benzoate, carbonic anhydride, 2,2,2-trichloroethyl carbonate (Troc), 2-trimethylsilyl ethyl carbonate), silyl ethers (e.g., trimethylsilyl (TMS), triethylsilyl (TES), triisopropylsilyl (TIPS), triphenylsilyl (TPS), tert-butyldimethylsilyl (TBDMS), tert-butyldiphenylsilyl (TBDPS)), etc. When the part is a nitrogen atom (thus forming a protected amine), exemplary protecting groups include benzyl (e.g., p-methoxyphenyl (PMP), 3,4-dimethoxybenzyloxy (PMB)), esters (e.g., benzoate), carbonyl (e.g., p-methoxybenzyl carbonyl (Moz), tert-butoxycarbonyl (BOC), 9-fluorenylmethoxycarbonyl (FMOC)), acetyl, carbamate, n-silyl, etc. Various protecting groups and their synthetic methods can be referred to in “Greene's Protective Groups in Organic Synthesis” (4th edition) by P. G. M. Wuts and T. W. Greene, John Wiley & Sons, Inc.

The term “substituted hydrocarbon” part used herein refers to a hydrocarbon part that is substituted by at least one non-carbon atom, including parts where carbon chain atoms are replaced by heteroatoms such as nitrogen, oxygen, silicon, phosphorus, boron, or halogen, and parts where the carbon chain includes additional substituents. These substituents include alkyl, alkoxy, acyl, acyloxy, alkene, alkenoxy, aryl, aryloxy, amino, amido, acetal, aminocarbonyl, carbocyclic, cyano, ester, ether, halogen, heterocycle, hydroxyl, ketone, ketal, phosphate, nitro, and thio.

The terms “comprising,” “including,” and “having” are intended to be inclusive and mean that there may be additional elements other than the listed elements. After the detailed description of the invention, it will be apparent that modifications and variations can be made without departing from the scope defined in the claims.

Other features and advantages of the invention will be set forth in the following description, and, in part, will be apparent from the description, or may be learned by practice of the invention. The objectives and other advantages of the invention may be realized and attained by the structure particularly pointed out in the written description, claims, and drawings.

### EXAMPLES

The following will provide a further detailed description of the general formula compounds of this invention, their preparation methods, and applications in conjunction with specific embodiments. The following examples are provided for illustrative purposes and to explain this invention. They should not be interpreted as limiting the scope of protection for this invention. All techniques realized based on the content described above of this invention are covered within the scope intended to be protected by this invention.

Unless otherwise specified, the raw materials and reagents used in the following examples are commercially available products or can be prepared by known methods.

This application employs the following abbreviations:
ACN: Acetonitrile
AIBN: Azobisisobutyronitrile
BBr₃: Boron tribromide
BPO: Benzoyl peroxide
CCl₄: Carbon tetrachloride
Cs₂CO₃: Cesium carbonate
DCM: Dichloromethane
DCE: 1,2-Dichloroethane
DIEA: N,N-Diisopropylethylamine
DMF: N,N-Dimethylformamide
EA: Ethyl acetate
EtOH: Ethanol
FA: Formic acid
K₂CO₃: Potassium carbonate
LAH: Lithium aluminium hydride
NaH: Sodium hydride
Na₂SO₄: Sodium sulfate
NaBH₄: Sodium borohydride
NaBH(OAc)₃: Sodium triacetoxyborohydride
NaF: Sodium fluoride
NBS: N-Bromosuccinimide
NCS: N-Chlorosuccinimide
PBr₃: Phosphorus tribromide
PE: Petroleum ether
t-BuOK: Potassium tert-butoxide
TBAB: Tetra-n-butylammonium bromide
TCCA: Trichloroisocyanuric acid
TEMED: N,N,N′,N′-Tetramethylethylenediamine
TEMPO: 2,2,6,6-Tetramethylpiperidine 1-oxyl
THF: Tetrahydrofuran

Compounds are named according to conventional nomenclature rules in the field, and commercially available reagents use supplier catalog names.

¹H NMR data were collected and recorded using a Bruker Avance Neo 400 MHz liquid superconducting nuclear magnetic resonance spectrometer at 400 MHz. CDCl₃, MeOD, DMSO-d₆ were used as solvents, and TMS (δ=0) was used as the internal standard to report chemical shift δ values (ppm). The mass spectrum was collected and recorded using Waters ACQUITY UPLC, and detected using ACQUITY UPLC BEH C8, 50 mm*2.1 mm, 1.7 μm (20180306-C8-08) chromatographic column. Mobile phase A: 0.01% TFA/H₂O; mobile phase B: CH₃CN; flow rate: 0.2 mL/min; column temperature: 30° C.; detection wavelength: UV-210 nm. High-performance liquid chromatography (HPLC) was measured by a Thermo UltiMate 3000 liquid chromatograph and a Venusil ASB C18 (4.6*250 mm, 5 μm) column was used for detection. Mobile phase A: phosphoric acid aqueous solution with pH=1.5; mobile phase B: CH₃CN; flow rate: 1.0 mL/min; column temperature: 35° C.; detection wavelength: UV-215 nm; injection volume: 2 μL; gradient elution conditions: The whole process is eluted at a flow rate of 1.0 mL/min, first eluting with 95% A and 5% B for 10 min, then eluting with 20% A and 80% B for 5 min, and finally eluting with 95% A and 5% B. 5% B elutes for 5 min. The percentage is the volume percentage of the mobile phase in the elution solution.

### Example 1: Preparation of Intermediate I

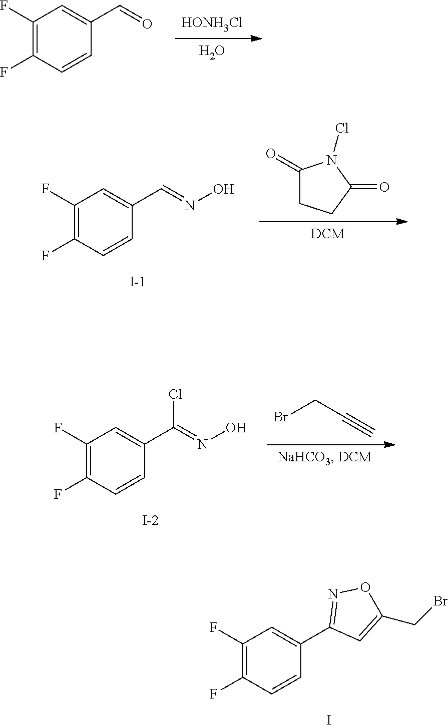

Hydroxylamine hydrochloride (366.91 g, 5.28 mol, 1.5 eq) was weighed and dissolved in 5 L of purified water. 3,4-Difluorobenzaldehyde (500.00 g, 3.52 mol, 1.0 eq) was added, and after 5 hours, the molecular weight of I-1 was detected by LC-MS.

The reaction mixture was filtered under vacuum and washed with purified water. The filter cake was dissolved in DCM and separated. The organic phase was dried over anhydrous Na₂SO₄, filtered, and kept as a reaction mixture.

N-Chlorosuccinimide (466.54 g, 3.52 mol, 1.0 eq) was added to the reaction mixture. After 8 hours, TLC showed no remaining 1-1. The reaction mixture was washed with purified water, separated, and the organic phase was dried over anhydrous Na₂SO₄ and concentrated under reduced pressure. Crystallization with petroleum ether yielded 330 g of I-2.

1-2 (300.00 g, 1.57 mol, 1.0 eq) was dissolved in 3 L of DCM. NaHCO₃ (263.79 g, 3.14 mol, 2.0 eq) and propargyl bromide (196.28 g, 1.65 mol, 1.05 eq) were added. After 24 hours, the molecular weight of intermediate I was detected by LC-MS. The reaction mixture was filtered and concentrated under reduced pressure. Column chromatography (PE-EA=20:1) was used for elution to obtain 360 g of intermediate I, a pale yellow solid. HPLC purity was: 98.82%; MS m/z (ESI): 275.16[M+H]⁺; ¹H NMR (400 MHz, DMSO-d₆) δ 7.65-7.56 (m, 1H), 7.56-7.47 (m, 1H), 7.44-7.33 (m, 1H), 7.27 (s, 1H), 4.69 (s, 2H).

The preparation of compounds listed below is carried out with reference to Name Structure Data

| Name | Structure | Data |
|---|---|---|
| Inter- mediate II | 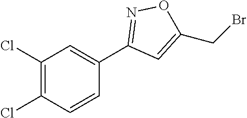 [SMILES: Clc1ccc(-c2cc(CBr)on2)cc1Cl] | ¹H NMR (400 MHZ, DMSO- d₆) δ 7.82 (d, J = 2.1 Hz, 1H), 7.66 (dd, J = 7.5, 2.0 Hz, 1H), 7.39 (d, J = 7.5 Hz, 1H), 7.26 (s, 1H), 4.69 (s, 2H). MS m/z(ESI): 307.52 [M + H]⁺. HPLC purity: 97.81%. |
| Inter- mediate III | 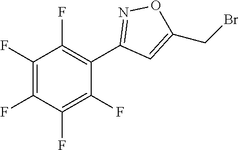 [SMILES: Fc1c(F)c(F)c(-c2cc(CBr)on2)c(F)c1F] | 1H NMR (400 MHZ, DMSO- d₆) δ 7.18 (s, 1H), 4.56 (s, 2H). MS m/z(ESI): 329.26 [M + H]⁺. HPLC purity: 98.21%. |
| Inter- mediate IV | 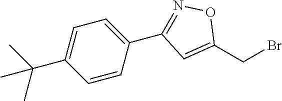 [SMILES: CC(C)(C)c1ccc(-c2cc(CBr)on2)cc1] | 1H NMR (400 MHZ, CDCl₃) δ 7.74 (d, J = 8.8 Hz, 2H), 7.49 (d, J = 8.8 Hz, 2H), 6.62 (s, 1H), 4.52 (s, 2H), 1.36 (s, 9H). MS m/z(ESI): 296.0 [M + H]⁺. HPLC purity: 97.1%. |
| Inter- mediate V | 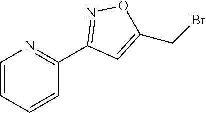 [SMILES: BrCc1cc(-c2ccccn2)no1] | MS m/z(ESI): 241.1 [M + H]⁺. HPLC purity: 94.7%. |
| Inter- mediate VI | 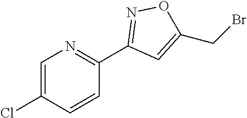 [SMILES: Clc1ccc(-c2cc(CBr)on2)nc1] | MS m/z(ESI): 275.2 [M + H]⁺. |
| Inter- mediate VII | 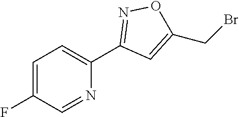 [SMILES: Fc1ccc(-c2cc(CBr)on2)nc1] | MS m/z(ESI): 257.2 [M + H]⁺. |

### Example 2: Preparation of Intermediate VIII

### Preparation of Intermediate VIII-2

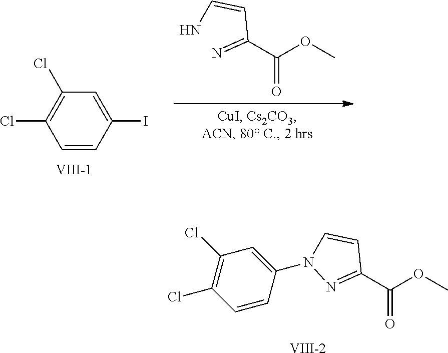

Methyl 1H-pyrazole-3-carboxylate (924 mg, 7.33 mmol, 1.00 eq) and VIII-1 (2.00 g, 7.33 mmol, 1.00 eq) were dissolved in acetonitrile (20.0 mL), followed by the addition of Cs₂CO₃ (4.78 g, 14.7 mmol, 2.00 eq), CuI (279 mg, 1.47 mmol, 0.20 eq), and N,N,N′,N′-tetramethylethylenediamine (170 mg, 1.47 mmol, 221 uL, 0.20 eq). The reaction mixture was stirred at 80° C. for 2 hours. LCMS showed the target product content to be 79.2% (m/z=271.1, M+H⁺). The mixture was concentrated under reduced pressure to obtain a crude product. The crude product was diluted with 20.0 mL of water and extracted with 20.0 mL of ethyl acetate, followed by washing the organic phase with 60.0 mL (20.0 mL×3) of saturated brine. The organic phase was dried over Na₂SO₄, filtered, and concentrated. Column chromatography purification (SiO₂, PE/EA=3:1, R_{f}=0.45) yielded a pale yellow solid intermediate VIII-2 (1.17 g, 4.04 mmol, yield 55.1%, purity 93.7%).

LCMS: m/z=271.1, [M+H]⁺.

¹H NMR (400 MHz, CDCl₃) b 7.94 (d, J=2.4 Hz, 1H), 7.92 (d, J=2.4 Hz, 1H), 7.54-7.62 (m, 2H), 7.03 (d, J=2.4 Hz, 1H), 3.98 (s, 3H).

### Preparation of Intermediate VIII-3

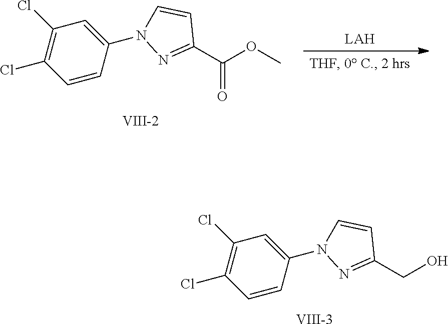

Intermediate VIII-2 (1.17 g, 4.04 mmol, 93.7% purity, 1.00 eq) was dissolved in THE (10.0 mL) and at 0° C., LAH (2.5 M, 2.10 mL, 1.30 eq) was added. The mixture was stirred for 2 hours. TLC monitoring (petroleum ether/ethyl acetate=3:1) confirmed the complete reaction of intermediate VIII-2 (R_{f}=0.45) and the formation of a new spot (R_{f}=0.01). At 0° C., the reaction was quenched with 0.2 mL of water, dropwise addition of NaOH (0.20 mL), followed by the addition of H₂O (0.60 mL). The reaction mixture was filtered and concentrated to obtain a pale yellow solid intermediate VIII-3 (0.99 g, 3.80 mmol, yield 94.0%, purity 93.4%).

LCMS: m/z=243.0, [M+H]⁺.

¹H NMR: (400 MHz, CDCl₃) δ 7.85 (d, J=2.4 Hz, 1H), 7.83 (s, 1H), 7.51 (s, 1H), 6.49 (d, J=2.8 Hz, 1H), 4.78 (s, 2H).

### Preparation of Intermediate VIII

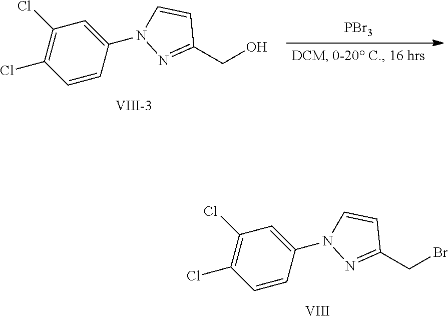

Intermediate VIII-3 (200 mg, 768 μmol, 93.4% purity, 1.00 eq) was dissolved in DCM (2.00 mL), and at 0° C., PBr₃ (208 mg, 768 μmol, 1.00 eq) was added dropwise. The mixture was stirred at 20° C. for 16 hours, and LCMS showed the target product content to be 91.9% (m/z=307.0, M+H+2). At 0° C., the reaction was quenched by the addition of NaHCO₃ (2.00 mL). The mixture was filtered, and additional NaHCO₃ (5.00 mL) and DCM (5.00 mL) were used for extraction. The organic phase was washed with 5.00 mL of saturated brine, dried over Na₂SO₄, filtered, and concentrated under reduced pressure to obtain a grey-white solid intermediate VIII (230 mg, 720 μmol, yield 93.7%, purity 95.8%).

LCMS: m/z=307.0, [M+H⁺2]

¹H NMR: (400 MHz, CDCl₃) δ 7.84 (t, J=1.6 Hz, 2H), 7.52 (d, J=1.2 Hz, 2H), 6.56 (d, J=2.8 Hz, 1H), 4.56 (s, 2H).

The preparation of compounds listed below is carried out with reference to Example 2.

| Name | Structure | Data |
|---|---|---|
| Inter- mediate IX | 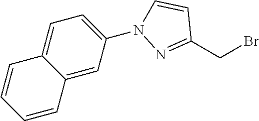 [SMILES: BrCc1ccn(-c2ccc3ccccc3c2)n1] | 1H NMR (400 MHZ, CDCl₃) δ 8.09 (d, J = 2.0 Hz, 1H), 8.02 (d, J = 2.4 Hz, 1H), 7.94 (d, J = 9.2 Hz, 1H), 7.86-7.88 (m, 3H), 7.51-7.54 (m, 2H), 6.59 (d, J = 2.4 Hz, 1H), 4.63 (s, 1H). MS m/z(ESI): 287.1 [M + H]⁺. HPLC purity: 99.5%. |
| Inter- mediate X | 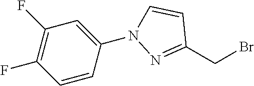 [SMILES: Fc1ccc(-n2ccc(CBr)n2)cc1F] | MS m/z(ESI): 270.5 [M + H]⁺. |

### Example 3: Preparation of Compound 1

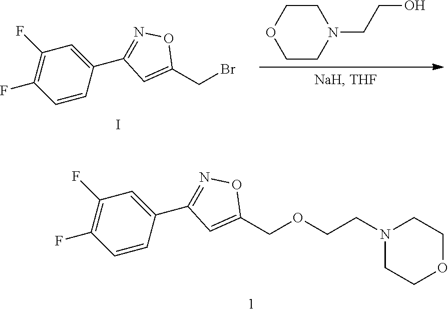

2-Morpholinoethanol (286 mg, 2.18 mmol, 1.2 eq) was weighed and dissolved in 15 mL of THF. NaH (146 mg, 3.64 mmol, 2.0 eq) and intermediate I (500 mg, 1.82 mmol, 1.0 eq) were added. After 5 hours, the molecular weight of Compound 1 was detected by LC-MS. The reaction mixture was washed with saturated brine and extracted with ethyl acetate (EA). The organic phase was dried over anhydrous Na₂SO₄, concentrated under reduced pressure, and purified by column chromatography (PE:EA=1:1) to yield 460 mg of Compound 1 as a white solid. HPLC purity: 98.77%; MS m/z (ESI): 325.26 [M+H]⁺; ¹H NMR (400 MHz, DMSO-d₆) δ 7.66-7.48 (m, 2H), 7.42-7.34 (m, 1H), 6.80 (s, 1H), 4.67 (s, 2H), 3.95-3.35 (m, 7H), 2.77-12.23 (m, 7H).

The preparation of compounds listed below is carried out with reference to Example 3

| Name | Structure | Data |
|---|---|---|
| Compound 2 | 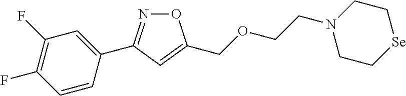 [SMILES: Fc1ccc(-c2cc(COCCN3CC[Se]CC3)on2)cc1F] | ¹H NMR (400 MHz, DMSO-d6) δ 7.57-7.49 (m, 1H), 7.47-7.40 (m, 1H), 6.82 (s, 0H), 4.70 (s, 1H), 3.47 (t, J = 6.5 Hz, 1H), 3.14- 2.99 (m, 1H), 2.76-2.63 (m, 1H), 2.59-2.44 (m, 3H). MS m/z(ESI): 389.15 [M + H]⁺. |
| | | HPLC purity: 95.02%. |
| Compound 3 | 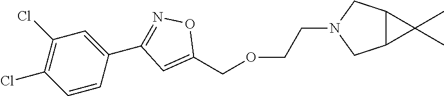 [SMILES: CC1(C)C2CN(CCOCc3cc(-c4ccc(Cl)c(Cl)c4)no3)CC21] | ¹H NMR (400 MHz, DMSO-d₆) δ 7.76 (d, J = 1.9 Hz, 1H), 7.67- 7.61 (m, 1H), 7.49 (d, J = 8.3 Hz, 1H), 6.87 (s, 1H), 4.01-3.41 (m, 2H), 2.95-2.72 (m, 4H), 2.71- 2.47 (m, 2H), 1.84-1.52 (m, 2H), 0.97 (d, J = 20.1 Hz, 5H). |
| | | MS m/z(ESI): 381.28 [M + H]⁺. |
| | | HPLC purity: 97.12%. |
| Compound 4 | 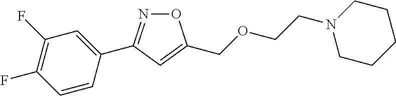 [SMILES: Fc1ccc(-c2cc(COCCN3CCCCC3)on2)cc1F] | ¹H NMR (400 MHz, DMSO-d₆) δ 7.67-7.51 (m, 2H), 7.43-7.27 (m, 1H), 6.79 (s, 1H), 4.69 (s, 2H), 3.81-3.39 (m, 2H), 2.99-2.79 (m, 2H), 2.61-2.26 (m, 5H), 1.64- 1.46 (m, 5H), 1.40 (m, J = 12.2, 7.0, 5.7, 1.5 Hz, 2H). |
| | | MS m/z(ESI): 323.30 [M + H]⁺. |
| | | HPLC purity: 98.03%. |
| Compound 5 | 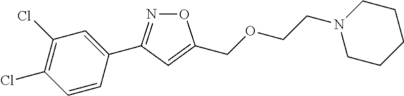 [SMILES: Clc1ccc(-c2cc(COCCN3CCCCC3)on2)cc1Cl] | ¹H NMR (400 MHz, DMSO-d₆) δ 7.77 (d, J = 1.9 Hz, 1H), 7.63 (dd, J = 8.4, 1.9 Hz, 1H), 7.49 (d, J = 8.3 Hz, 1H), 6.87 (s, 1H), 4.69 (s, 2H), 3.68-3.60 (m, 2H), 2.92- 2.84 (m, 2H), 2.52-2.41 (m, 4H), 1.63-1.35 (m, 7H). |
| | | MS m/z(ESI): 355.27 [M + H]⁺. |
| | | HPLC purity: 97.56%. |
| Compound 6 | 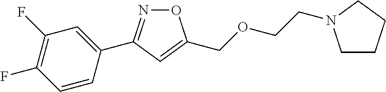 [SMILES: Fc1ccc(-c2cc(COCCN3CCCC3)on2)cc1F] | ¹H NMR (400 MHz, DMSO-d₆) δ 7.64-7.50 (m, 2H), 7.43-7.33 (m, 1H), 6.79 (s, 1H), 4.67 (s, 2H), 3.62-3.54 (m, 2H), 3.00- 2.90 (m, 2H), 2.90-2.80 (m, 4H), 2.01-1.83 (m, 4H). |
| | | MS m/z(ESI): 309.23 [M + H]⁺. |
| | | HPLC purity: 96.91%. |
| Compound 7 | 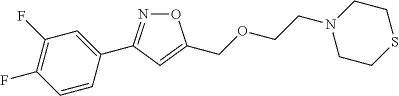 [SMILES: Fc1ccc(-c2cc(COCCN3CCSCC3)on2)cc1F] | ¹H NMR (400 MHz, DMSO-d₆) δ 7.62-7.50 (m, 2H), 7.43-7.33 (m, 1H), 6.81 (s, 1H), 4.71 (s, 2H), 3.68-3.60 (m, 2H), 2.83- 2.67 (m, 9H), 2.55 (t, J = 6.4 Hz, 2H). MS m/z(ESI): 341.23 [M + H]⁺. |
| | | HPLC purity: 99.07%. |
| Compound 8 | 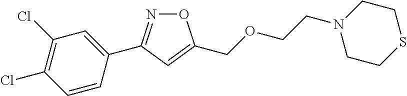 [SMILES: Clc1ccc(-c2cc(COCCN3CCSCC3)on2)cc1Cl] | ¹H NMR (400 MHz, DMSO-d₆) δ 7.77 (d, J = 1.9 Hz, 1H), 7.63 (dd, J = 8.4, 1.9 Hz, 1H), 7.46 (d, J = 8.4 Hz, 1H), 6.87 (s, 1H), 4.71 (s, 2H), 3.64 (t, J = 6.4 Hz, 2H), 2.83- 2.67 (m, 9H), 2.56 (t, J = 6.5 |
| | | Hz, 2H). |
| | | MS m/z(ESI): 373.20 [M + H]⁺. |
| | | HPLC purity: 99.37%. |
| Compound 9 | 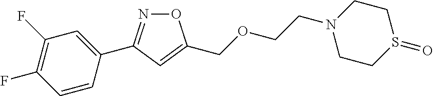 [SMILES: O=S1CCN(CCOCc2cc(-c3ccc(F)c(F)c3)no2)CC1] | ¹H NMR (400 MHz, DMSO-d₆) δ 7.62-7.50 (m, 2H), 7.43-7.33 (m, 1H), 6.81 (s, 1H), 4.68 (s, 2H), 3.74-3.65 (m, 2H), 2.97- 2.90 (m, 4H), 2.89-2.80 (m, 2H), 2.70-2.63 (m, 4H). MS m/z(ESI): 357.26 [M + H]⁺. |
| | | HPLC purity: 96.06%. |
| Compound 10 | 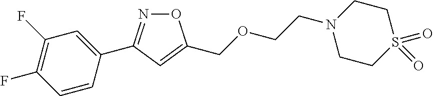 [SMILES: O=S1(=O)CCN(CCOCc2cc(-c3ccc(F)c(F)c3)no2)CC1] | ¹H NMR (400 MHz, DMSO-d₆) δ 7.65-7.51 (m, 2H), 7.43-7.33 (m, 1H), 6.82 (s, 1H), 4.70 (s, 2H), 3.73-3.55 (m, 2H), 3.15- 3.08 (m, 4H), 3.04-2.95 (m, 6H). MS m/z(ESI): 373.24 [M + H]⁺. |
| | | HPLC purity: 97.17%. |
| Compound 11 | 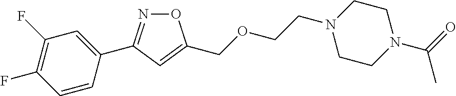 [SMILES: CC(=O)N1CCN(CCOCc2cc(-c3ccc(F)c(F)c3)no2)CC1] | ¹H NMR (400 MHz, DMSO-d₆) δ 7.62-7.51 (m, 2H), 7.43-7.33 (m, 1H), 6.82 (s, 1H), 4.71 (s, 2H), 3.60-3.42 (m, 7H), 2.63- 2.50 (m, 7H), 2.01 (s, 3H). MS m/z(ESI): 366.29 [M + H]⁺. HPLC purity: 98.22%. |
| Compound 12 | 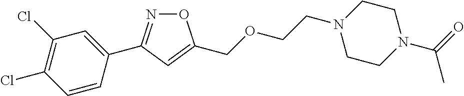 [SMILES: CC(=O)N1CCN(CCOCc2cc(-c3ccc(Cl)c(Cl)c3)no2)CC1] | ¹H NMR (400 MHz, DMSO-d₆) δ 7.76 (d, J = 2.2 Hz, 1H), 7.64 (dd, J = 8.4, 2.3 Hz, 1H), 7.52 (d, J = 8.3 Hz, 1H), 6.87 (s, 1H), 4.71 (s, 2H), 3.65-3.57 (m, 2H), 3.56- 3.42 (m, 5H), 2.62-2.51 (m, 7H), 2.01 (s, 3H). |
| | | MS m/z(ESI): 398.21 [M + H]⁺. |
| | | HPLC purity: 97.91%. |
| Compound 13 | 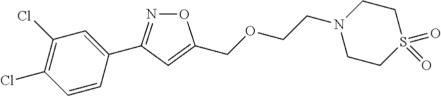 [SMILES: O=S1(=O)CCN(CCOCc2cc(-c3ccc(Cl)c(Cl)c3)no2)CC1] | ¹H NMR (400 MHz, DMSO-d₆) δ 7.76 (d, J = 2.2 Hz, 1H), 7.64 (dd, J = 8.4, 2.3 Hz, 1H), 7.49 (d, J = 8.3 Hz, 1H), 6.87 (s, 1H), 4.69 (s, 2H), 3.75-3.67 (m, 2H), 3.12 (dd, J = 5.6, 4.4 Hz, 4H), 3.06- 2.91 (m, 7H). |
| | | MS m/z(ESI): 405.18 [M + H]⁺. |
| | | HPLC purity: 98.05%. |
| Compound 14 | 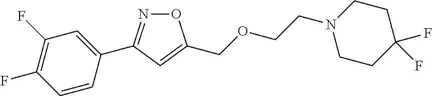 [SMILES: Fc1ccc(-c2cc(COCCN3CCC(F)(F)CC3)on2)cc1F] | ¹H NMR (400 MHz, DMSO-d₆) δ 7.62-7.51 (m, 2H), 7.43-7.33 (m, 1H), 6.81 (s, 1H), 4.68 (s, 2H), 3.74-3.66 (m, 2H), 3.00- 2.92 (m, 2H), 2.81-2.65 (m, 4H), 2.32-2.19 (m, 3H), 2.18 (dd, J = 5.6, 5.0 Hz, 1H). |
| | | MS m/z(ESI): 359.26 [M + H]⁺. |
| | | HPLC purity: 95.03%. |
| Compound 15 | 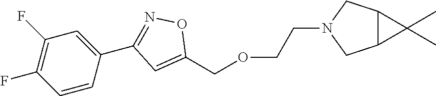 [SMILES: CC1(C)C2CN(CCOCc3cc(-c4ccc(F)c(F)c4)no3)CC21] | ¹H NMR (400 MHz, DMSO-d₆) δ 7.62-7.50 (m, 1H), 7.43-7.33 (m, 0H), 6.82 (s, 0H), 4.70 (d, J = 0.7 Hz, 1H), 3.78-3.60 (m, 1H), 3.01-2.87 (m, 1H), 2.72-2.64 (m, 1H), 2.57-2.44 (m, 1H), 1.67-1.55 (m, 1H), 0.99 (s, 1H), |
| | | 0.94 (s, 1H). |
| | | MS m/z(ESI): 349.31 [M + H]⁺. |
| | | HPLC purity: 96.67%. |
| Compound 21 | 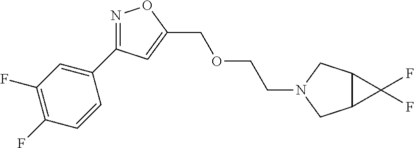 [SMILES: Fc1ccc(-c2cc(COCCN3CC4C(C3)C4(F)F)on2)cc1F] | ¹H NMR (400 MHz, DMSO-d₆) δ 7.62-7.50 (m, 2H), 7.43-7.33 (m, 1H), 6.81 (s, 1H), 4.75-4.62 (m, 2H), 3.70 (t, J = 6.5 Hz, 2H), 3.14-3.06 (m, 2H), 2.73-2.65 (m, 2H), 2.51-2.31 (m, 4H). MS m/z(ESI): 357.27 [M + H]⁺. HPLC purity: 95.16%. |
| Compound 22 | 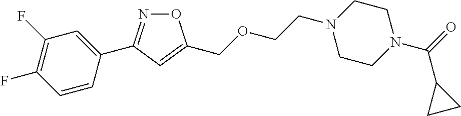 [SMILES: O=C(C1CC1)N1CCN(CCOCc2cc(-c3ccc(F)c(F)c3)no2)CC1] | ¹H NMR (400 MHz, DMSO-d₆) δ 7.61-7.50 (m, 2H), 7.43-7.32 (m, 1H), 6.81 (s, 1H), 4.69 (s, 2H), 3.58-3.45 (m, 6H), 2.62- 2.49 (m, 6H), 1.69-1.57 (m, 1H), 0.91-0.84 (m, 4H). MS m/z(ESI): 392.33 [M + H]⁺. HPLC purity: 96.28%. |
| Compound 23 | 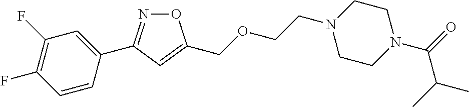 [SMILES: CC(C)C(=O)N1CCN(CCOCc2cc(-c3ccc(F)c(F)c3)no2)CC1] | ¹H NMR (400 MHz, DMSO-d₆) δ 7.61-7.50 (m, 2H), 7.43-7.33 (m, 1H), 6.81 (s, 1H), 4.69 (s, 2H), 3.60-3.50 (m, 2H), 3.54- 3.41 (m, 4H), 2.80-2.66 (m, 1H), 2.62-2.49 (m, 6H), 1.07 (d, J = 7.1 Hz, 6H). |
| | | MS m/z(ESI): 394.35 [M + H]⁺. |
| | | HPLC purity: 97.73%. |
| Compound 24 | 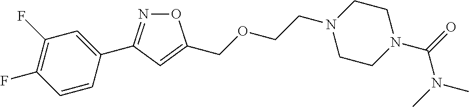 [SMILES: CN(C)C(=O)N1CCN(CCOCc2cc(-c3ccc(F)c(F)c3)no2)CC1] | ¹H NMR (400 MHz, DMSO-d₆) δ 7.60-7.50 (m, 2H), 7.43-7.32 (m, 1H), 6.92 (s, 1H), 4.69 (s, 2H), 3.58-3.50 (m, 2H), 3.49- 3.38 (m, 4H), 2.95 (s, 5H), 2.58- 2.49 (m, 2H), 2.45-2.37 (m, 4H). |
| | | MS m/z(ESI): 395.34 [M + H]⁺. |
| | | HPLC purity: 97.03%. |
| Compound 33 | 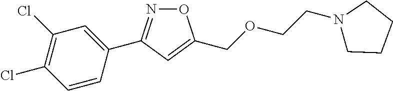 [SMILES: Clc1ccc(-c2cc(COCCN3CCCC3)on2)cc1Cl] | ¹H NMR (400 MHz, DMSO-d₆) δ 7.82 (d, J = 2.0 Hz, 1H), 7.65 (dd, J = 7.5, 2.0 Hz, 1H), 7.39 (d, J = 7.5 Hz, 1H), 7.19 (s, 1H), 4.63 (s, 2H), 3.66 (t, J = 7.2 Hz, 2H), 3.28- 3.16 (m, 4H), 2.98 (t, J = 7.2 |
| | | Hz, 2H), 2.02-1.90 (m, 4H). |
| | | MS m/z(ESI): 341.23 [M + H]⁺. |
| | | HPLC purity: 96.91%. |
| Compound 54 | 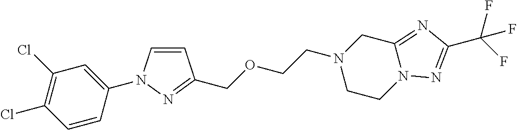 [SMILES: FC(F)(F)c1nc2n(n1)CCN(CCOCc1ccn(-c3ccc(Cl)c(Cl)c3)n1)C2] | ¹H NMR (400 MHz, CDCl₃) δ 7.86 (t, J = 2.4 Hz, 2H), 7.52 (s, 2H), 6.50 (d, J = 2.4 Hz, 1H), 4.64 (s, 2H), 4.29 (d, J = 4.4 Hz, 2H), 3.97 (s, 2H), 3.77 (t, J = 4.8 Hz, 2H), 3.17 (d, J = 4.4 Hz, 2H), 2.96 (t, J = 4.0 Hz, 2H). MS m/z(ESI): 460.9 [M + H]⁺. HPLC purity: 99.4%. |
| Compound 55 | 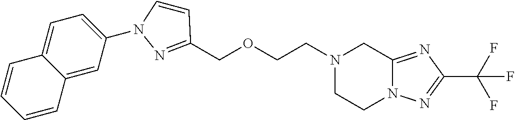 [SMILES: FC(F)(F)c1nc2n(n1)CCN(CCOCc1ccn(-c3ccc4ccccc4c3)n1)C2] | ¹H NMR (400 MHz, CDCl₃) δ 8.21 (d, J = 2.0 Hz, 1H), 7.98 (s, 1H), 7.94 (d, J = 12.4 Hz, 1H), 7.90 (t, J = 6.0 Hz, 3H), 7.54-7.56 (m, 2H), 7.07 (s, 1H), 4.00 (s, 3H). MS m/z(ESI): 253.2 [M + H]⁺. HPLC purity: 97.4%. |
| Compound 56 | 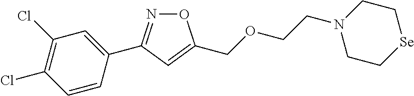 [SMILES: Clc1ccc(-c2cc(COCCN3CC[Se]CC3)on2)cc1Cl] | ¹H NMR (400 MHz, CDCl₃) δ 7.92 (d, J = 2.0 Hz, 1H), 7.65-7.67 (m, 2H), 7.55 (d, J = 8.0 Hz, 2H), 6.59 (s, 1H), 4.69 (s, 2H),3.81- 3.85 (m, 2H), 2.74-3.13 (m, 10H). MS m/z(ESI): 421.1 [M + H]⁺. |
| | | HPLC purity: 93.3%. |
| Compound 57 | 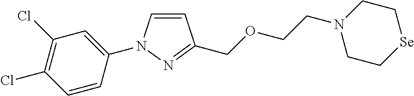 [SMILES: Clc1ccc(-n2ccc(COCCN3CC[Se]CC3)n2)cc1Cl] | ¹H NMR (400 MHz, CDCl₃) δ 7.51- 7.88 (m, 2H), 7.51 (s, 2H), 6.50 (t, J = 1.6 Hz, 1H), 4.62 (s, 2H), 3.81 (s, 2H), 3.20 (s, 4H), 2.91 (s, 6H). MS m/z(ESI): 419.8, [M + H]⁺. HPLC purity: 98.5%. |
| Compound 60 | 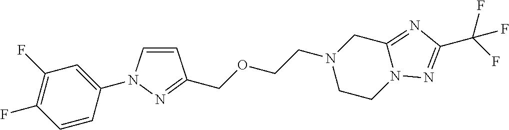 [SMILES: Fc1ccc(-n2ccc(COCCN3CCn4nc(C(F)(F)F)nc4C3)n2)cc1F] | ¹H NMR (400 MHz, CDCl₃) δ 7.83 (t, J = 2.0 Hz, 1H), 7.39-7.58 (m, 1H), 7.37-7.39 (m, 1H), 7.24- 7.28 (m, 1H), 6.46-6.53 (m, 1H), 4.64 (s, 2H), 4.25 (s, 2H), 3.93 (s, 2H), 3.75 (t, J = 4.8 Hz, 2H), 3.13 (d, J = 5.2 Hz, 2H), 2.89 (d, J = 4.4 Hz, 2H).. MS m/z(ESI): 429.1 [M + H]⁺. |
| | | HPLC purity: 97.9%. |
| Compound 61 | 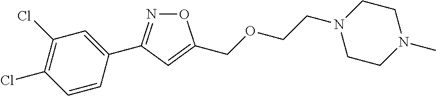 [SMILES: CN1CCN(CCOCc2cc(-c3ccc(Cl)c(Cl)c3)no2)CC1] | ¹H NMR (400 MHz, CDCl₃) δ 7.88 (d, J = 2.0 Hz, 1H), 7.62-7.64 (m, 1H), 7.52 (d, J = 8.4 Hz, 1H), 6.54 (s, 1H), 4.66 (s, 1H), 3.69 (t, J = 5.6 Hz, 2H), 2.63 (t, J = 5.6 Hz, 2H), 2.54 (s, 8H), 2.28 (s, 3H). |
| | | MS m/z(ESI): 370.1 [M + H]⁺. |
| | | HPLC purity: 99.7%. |
| Compound 62 | 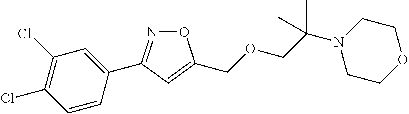 [SMILES: CC(C)(COCc1cc(-c2ccc(Cl)c(Cl)c2)no1)N1CCOCC1] | ¹H NMR (400 MHz, CDCl₃) δ 7.92 (d, J = 2.0 Hz, 1H), 7.65-7.68 (m, 1H), 7.55 (d, J = 8.4 Hz, 1H), 6.53 (s, 1H), 4.67 (s, 2H), 3.72- 3.76 (m, 4H), 3.45 (s, 2H), 2.62 (s, 4H), 1.09 (s, 6H). MS m/z(ESI): 385.9 [M + H]⁺. HPLC purity: 99.2%. |
| Compound 63 | 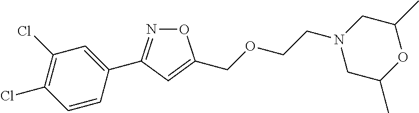 [SMILES: CC1CN(CCOCc2cc(-c3ccc(Cl)c(Cl)c3)no2)CC(C)O1] | ¹H NMR (400 MHz, CDCl₃) δ 7.91 (d, J = 2.0 Hz, 1H), 7.64-7.66 (m, 1H), 7.55 (d, J = 8.4 Hz, 1H), 6.55 (s, 1H), 4.69 (s, 2H), 3.75 (s, 4H), 2.66-2.83 (m, 4H), 1.82- 1.86 (m, 2H), 1.17 (d, J = 6.4 Hz, 6H). MS m/z(ESI): 385.2 [M + H]⁺. |
| | | HPLC purity: 98.9%. |
| Compound 66 | 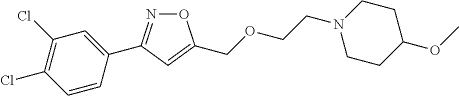 [SMILES: COC1CCN(CCOCc2cc(-c3ccc(Cl)c(Cl)c3)no2)CC1] | ¹H NMR (400 MHz, CDCl₃) δ 7.91 (d, J = 1.6 Hz, 1H), 7.65-7.68 (m, 1H), 7.55 (d, J = 8.4 Hz, 1H), 6.63 (s, 1H), 4.69 (s, 2H), 4.00- 4.02 (m, 2H), 3.59 (s, 1H), 3.50- 3.54 (m, 2H), 3.34-3.35 (m, 3H), 3.25-3.26 (m, 2H), 3.05-3.11 |
| | | (m, 2H), 2.34-2.17 (m, 2H), |
| | | 1.99-2.05 (m, 2H). |
| | | MS m/z(ESI): 385.1 [M + H]⁺. |
| | | HPLC purity: 97.9%. |
| Compound 67 | 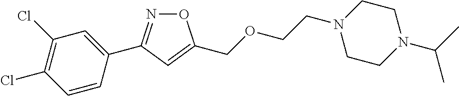 [SMILES: CC(C)N1CCN(CCOCc2cc(-c3ccc(Cl)c(Cl)c3)no2)CC1] | ¹H NMR (400 MHz, CDCl₃) δ 7.91 (d, J = 1.6 Hz, 1H), 7.64-7.67 (m, 1H), 7.55 (d, J = 8.4 Hz, 1H), 6.62 (s, 1H), 4.70 (s, 2H), 3.95- 3.97 (m, 2H), 3.43-3.72 (m, 10H), 3.24-3.32 (m, 3H), 1.40 (d, J = 6.8 Hz, 6H). |
| | | MS m/z(ESI): 398.2 [M + H]⁺. |
| | | HPLC purity: 96.7%. |
| Compound 68 | 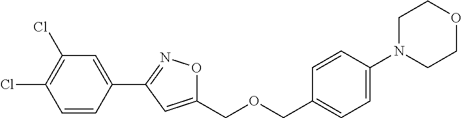 [SMILES: Clc1ccc(-c2cc(COCc3ccc(N4CCOCC4)cc3)on2)cc1Cl] | ¹H NMR (400 MHz, CDCl₃) δ 7.91 (d, J = 2.0 Hz, 1H), 7.64-7.67 (m, 1H), 7.54 (d, J = 8.4 Hz, 1H), 7.29 (d, J = 8.4 Hz, 1H), 6.92 (d, J = 8.4 Hz, 1H), 6.54 (s, 1H), 4.64 (s, 2H), 4.58 (s, 2H), 3.87 (t, J = 4.8 Hz, 4H), 3.18 (t, J = 4.8 Hz, 4H). |
| | | MS m/z(ESI): 419.0 [M + H]⁺. |
| | | HPLC purity: 98.9%. |
| Compound 69 | 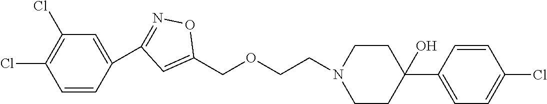 [SMILES: OC1(c2ccc(Cl)cc2)CCN(CCOCc2cc(-c3ccc(Cl)c(Cl)c3)no2)CC1] | ¹H NMR (400 MHz, CDCl₃) δ 7.93 (d, J = 1.6 Hz, 1H), 7.65-7.67 (m, 1H), 7.55 (d, J = 8.4 Hz, 1H), 7.36-7.43 (m, 4H), 6.64 (s, 1H), 4.71 (s, 2H), 4.04-4.05 (m, 2H), 3.67 (d, J = 11.2 Hz, 2H), 3.33- 3.41 (m, 4H), 2.58-2.65 (m, |
| | | 2H), 1.90-1.94 (m, 2H). |
| | | MS m/z(ESI): 481.3 [M + H]⁺. |
| | | HPLC purity: 96.9%. |
| Compound 71 | 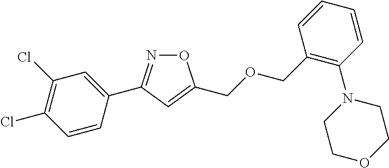 [SMILES: Clc1ccc(-c2cc(COCc3ccccc3N3CCOCC3)on2)cc1Cl] | ¹H NMR (400 MHz, CDCl₃) δ 7.91 (d, J = 2.0 Hz, 1H), 7.65-7.67 (m, 1H), 7.55 (d, J = 8.4 Hz, 1H), 7.48-7.50 (m, 1H), 7.31-7.36 (m, 1H), 7.12-7.18 (m, 2H), 6.56 (s, 1H), 4.76 (s, 2H), 4.72 (s, 2H), 3.82 (t, J = 4.4 Hz, 4H), 2.94 (t, J = 4.4 Hz, 4H). MS m/z(ESI): 419.0 [M + H]⁺. HPLC purity: 98.0%. |
| Compound 72 | 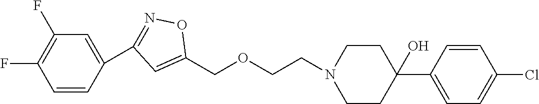 [SMILES: OC1(c2ccc(Cl)cc2)CCN(CCOCc2cc(-c3ccc(F)c(F)c3)no2)CC1] | ¹H NMR (400 MHz, CDCl₃) δ 8.36 (s, 1H), 7.54-7.68 (m, 1H), 7.45- 7.54 (m, 1H), 7.39 (d, J = 26.4 Hz, 2H), 7.32 (d, J = 15.2 Hz, 2H), 6.60 (s, 1H), 4.70 (s, 2H), 3.97-3.99 (m, 2H), 3.40-3.41 (m, 2H), 3.12-3.15 (m, 4H), 2.47- |
| | | 2.54 (m, 2H), 1.85 (d, J = 13.6 |
| | | Hz, 2H). |
| | | MS m/z(ESI): 449.3 [M + H]⁺. |
| | | HPLC purity: 93.1%. |
| Compound 75 | 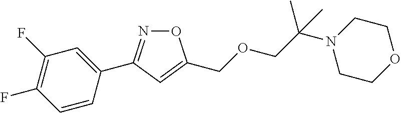 [SMILES: CC(C)(COCc1cc(-c2ccc(F)c(F)c2)no1)N1CCOCC1] | ¹H NMR (400 MHz, CDCl₃) δ 7.64-7.69 (m, 1H), 7.53-7.56 (m, 1H), 7.23-7.25 (m, 1H), 6.51 (s, 1H), 4.67 (s, 2H), 3.72 (s, 4H), 3.45 (s, 2H), 2.62 (s, 4H), 1.09 (s, 6H). MS m/z(ESI): 353.3 [M + H]⁺. HPLC purity: 95.3%. |

### Example 4: Preparation of Compound 74

### Preparation of Compound 74a

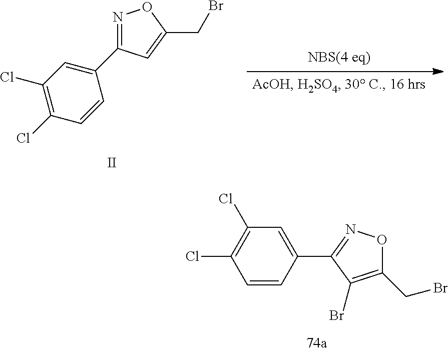

Intermediate II (1.00 g, 3.26 mmol, 1.00 eq) was dissolved in acetic acid, and NBS (2.32 g, 13.0 mmol, 4.00 eq) and H₂SO₄ (1.92 g, 19.5 mmol, 1.04 mL, 6.00 eq) were added. The mixture was stirred at 30° C. for 16 hours. LCMS showed the target molecule content to be 98.2% (m/z=385.8, M+H⁺). The reaction was quenched with NaHCO₃ (20.0 mL) at 10° C., diluted with 20.0 mL of water, and extracted with 20.0 mL of ethyl acetate. The organic phase was washed with 60.0 mL (20.0 mL×3) of saturated brine, dried over Na₂SO₄, filtered, and concentrated under reduced pressure. Column chromatography purification (SiO₂, PE/EA=10/1, R_{f}=0.46) of the concentrate yielded a pale-yellow oily compound 74a (1.12 g, 2.90 mmol, yield 79.5%, purity 100%).

LCMS m/z=385.8, M+H⁺

¹H NMR: 400 MHz, CDCl₃ δ 8.21 (d, J=2.0 Hz, 1H), 7.98 (s, 1H), 7.94 (d, J=12.4 Hz, 1H), 7.90 (t, J=6.0 Hz, 3H), 7.54-7.56 (m, 2H), 7.07 (s, 1H), 4.00 (s, 3H).

### Preparation of Compound 74

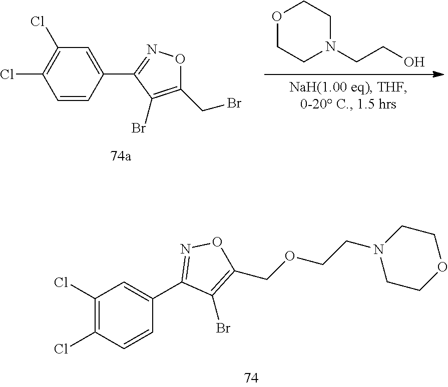

2-Morpholinoethanol (20.4 mg, 155 μmol, 19.0 μL, 1.20 eq) was dissolved in THE (0.50 mL) and at 0° C., NaH (5.70 mg, 142 μmol, 60.0% content, 1.00 eq) was added. The reaction was stirred for 0.5 hours. Compound 74a (50.0 mg, 129 μmol, 1.00 eq) was then added. The mixture was stirred at 20° C. for 1 hour. LCMS showed the formation of 82.5% of the target product (m/z=437.0, M+H⁺). The reaction mixture was quenched by slowly adding NH₄Cl (2.00 mL) at 10° C. and stirred for 0.5 hours, diluted with 2.00 mL H₂O, and extracted with 2.00 mL of ethyl acetate. The organic phase was washed with 6.00 mL of saturated brine (2.00 mL×3), dried over Na₂SO₄, filtered, and concentrated under reduced pressure to obtain a concentrate. Purification (FA condition, column: Phenomenex Luna C18 150×25 mm×10 um; mobile phase: [water (FA)-ACN]; B %: 21%-51%, 10 min) yielded a yellow solid compound 74 (22.0 mg, 50.2 μmol, yield 38.8%, purity 99.7%).

LCMS m/z=437.0, M+H⁺

¹H NMR: 400 MHz, CDCl₃ δ 7.99 (d, J=2.4 Hz, 1H), 7.72-7.74 (m, 1H), 7.59 (d, J=8.4 Hz, 1H), 4.71 (s, 2H), 3.79 (t, J=4.8 Hz, 6H), 2.74 (t, J=5.6 Hz, 2H), 2.65 (s, 4H).

### Example 5: Preparation of Compound 64

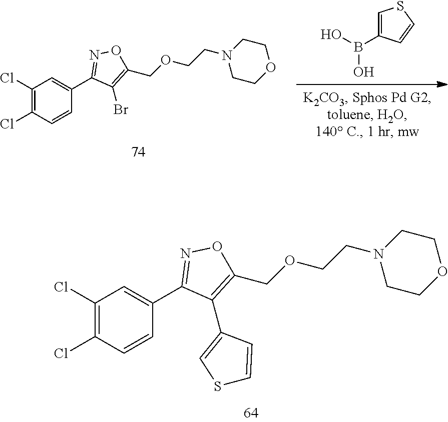 [SMILES: Clc1ccc(-c2noc(COCCN3CCOCC3)c2Br)cc1Cl ; OB(O)c1ccsc1 ; "K2CO3, Sphos Pd G2,toluene, H2O,140° C., 1 hr, mw" ; Clc1ccc(-c2noc(COCCN3CCOCC3)c2-c2ccsc2)cc1Cl]

Compound 74 (100 mg, 229 μmol, 1.00 eq), 3-thiopheneboronic acid (32.2 mg, 252 μmol, 1.10 eq), Sphos Pd G2 (16.5 mg, 22.9 μmol, 0.10 eq), and K₂CO₃ (110 mg, 802 μmol, 3.50 eq) were added to a microwave reaction tube, along with toluene (1.00 mL) and water (0.20 mL). The mixture was heated in a microwave to 140° C. for 1 hour. LCMS showed the formation of 29.0% of the target product (m/z=439.1, M+H⁺). The reaction mixture was filtered and concentrated under reduced pressure to obtain a concentrate. Purification (FA condition, column: Phenomenex Luna C18 150×25 mm×10 μm; mobile phase: [water (FA)-ACN]; B %: 26%-56%, 58 min) yielded a yellow oily compound 64 (14.3 mg, 31.5 μmol, yield 13.7%, purity 96.8%).

LCMS: m/z=439.1, M+H⁺

¹H NMR: 400 MHz, CDCl₃ δ 7.65 (d, J=2.0 Hz, 1H), 7.44 (d, J=8.4 Hz, 1H), 7.39-7.41 (m, 1H), 7.35-7.36 (m, 1H), 7.28-7.30 (m, 1H), 6.91-6.92 (m, 1H), 4.62 (s, 2H), 3.72-3.76 (m, 6H), 2.65 (s, 2H), 2.54 (s, 4H).

### Example 6: Preparation of Compound 73

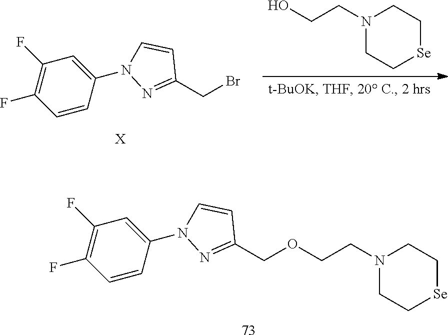

2-Selenomorpholinoethanol (71.0 mg, 366 μmol, 1.00 eq) was dissolved in THE (1.00 mL), followed by the addition of t-BuOK (1 M, 549 μL, 1.50 eq), and then intermediate X (100 mg, 366 μmol, 1.00 eq) was added. The reaction was stirred at 20° C. for 2 hours. LC-MS showed the formation of 58.4% of the target product (m/z=388.0, M+H⁺). The mixture was diluted with 2.00 mL H₂O, extracted with 2.00 mL EtOAc, and the organic phase was washed with 6.00 mL of saturated brine (2.00 mL×3), dried over Na₂SO₄, filtered, and concentrated under reduced pressure to obtain a concentrate. Purification (FA condition, column: Phenomenex Luna C18 150×25 mm×10 μm; mobile phase: [water (FA)-ACN]; B %: 7%-37%, 10 min) yielded a yellow gel-like compound 73 (87.7 mg, 207 μmol, yield 28.3%, purity 91.4%).

LCMS: m/z=388.0, M+H⁺

¹H NMR: 400 MHz, CDCl₃ δ 7.81 (d, J=2.0 Hz, 1H), 7.55-7.60 (m, 1H), 7.36-7.37 (m, 1H), 7.23-7.25 (m, 1H), 6.50 (d, J=2.4 Hz, 1H), 4.61 (s, 2H), 3.66 (t, J=6.4 Hz, 2 Hz), 2.96 (t, J=5.2 Hz, 4H), 2.69-2.75 (m, 6H).

### Example 7: Preparation of Compound 37

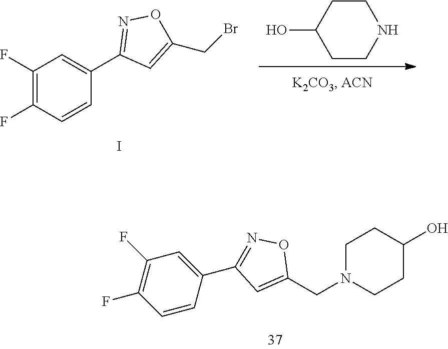

4-Hydroxypiperidine (221 mg, 2.18 mmol, 1.2 eq) was weighed and dissolved in 30 mL of ACN. Potassium carbonate (K₂CO₃, 503 mg, 3.64 mmol, 2.0 eq) and intermediate I (500 mg, 1.82 mmol, 1.2 eq) were added. After 5 hours, the molecular weight of compound 37 was detected by LC-MS. The reaction mixture was washed with saturated brine and extracted with ethyl acetate (EA). The organic phase was dried over anhydrous Na₂SO₄, concentrated under reduced pressure, and purified by column chromatography (PE:EA=1:1) to obtain 400 mg of compound 37, a yellow solid. HPLC purity: 96.86%; MS m/z (ESI): 295.23[M+H]⁺;

¹H NMR (400 MHz, DMSO-d₆) δ 8.02-7.90 (m, 1H), 7.82-7.67 (m, 1H), 7.64-7.52 (m, 1H), 4.58 (d, J=4.0 Hz, 1H), 3.69 (s, 2H), 3.52-3.40 (m, 1H), 2.85-2.61 (m, 1H), 2.25-2.10 (m, 2H), 1.86-1.58 (m, 2H), 1.52-1.31 (m, 2H).

The preparation of compounds listed below is carried out with reference to Example 7

| Name | Structure | Data |
|---|---|---|
| Compound 16 | 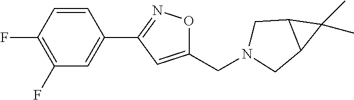 [SMILES: CC1(C)C2CN(Cc3cc(-c4ccc(F)c(F)c4)no3)CC21] | ¹H NMR (400 MHz, DMSO- d₆) δ 7.62-7.51 (m, 2H), 7.43-7.32 (m, 1H), 6.71 (s, 1H), 3.84 (s, 2H), 3.03- 2.90 (m, 2H), 2.47-2.34 (m, 2H), 1.82-1.70 (m, 2H), 0.98 (d, J = 19.9 Hz, |
| | | 5H). |
| | | MS m/z(ESI): 305.25 |
| | | [M + H]⁺. |
| | | HPLC purity: 97.49%. |
| Compound 17 | 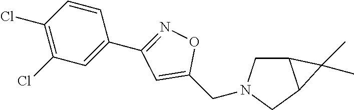 [SMILES: CC1(C)C2CN(Cc3cc(-c4ccc(Cl)c(Cl)c4)no3)CC21] | ¹H NMR (400 MHz, DMSO- d₆) δ 7.78 (d, J = 2.3 Hz, 1H), 7.63 (dd, J = 8.5, 2.2 Hz, 1H), 7.45 (d, J = 8.4 Hz, 1H), 6.76 (s, 1H), 3.84 (s, 2H), 3.03-2.90 (m, 2H), 2.47-2.34 (m, 2H), 1.83- |
| | | 1.71 (m, 2H), 0.98 (d, J = |
| | | 19.9 Hz, 5H). |
| | | MS m/z(ESI): 337.23 |
| | | [M + H]⁺. |
| | | HPLC purity: 98.04%. |
| Compound 18 | 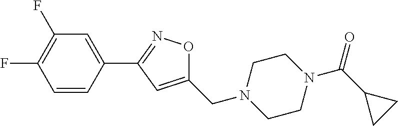 [SMILES: O=C(C1CC1)N1CCN(Cc2cc(-c3ccc(F)c(F)c3)no2)CC1] | ¹H NMR (400 MHz, DMSO- d₆) δ 7.62-7.49 (m, 2H), 7.43-7.33 (m, 1H), 6.72 (s, 1H), 3.95 (s, 2H), 3.56- 3.42 (m, 4H), 2.67-2.56 (m, 2H), 2.55-2.44 (m, 2H), 1.64 (p, J = 6.4 Hz, 1H), 0.95-0.77 (m, 4H). |
| | | MS m/z(ESI): 348.30 |
| | | [M + H]⁺. |
| | | HPLC purity: 96.93%. |
| Compound 19 | 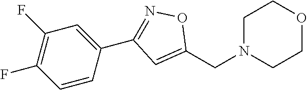 [SMILES: Fc1ccc(-c2cc(CN3CCOCC3)on2)cc1F] | ¹H NMR (400 MHz, DMSO- d₆) δ 7.61-7.49 (m, 2H), 7.43-7.33 (m, 1H), 6.70 (s, 1H), 3.93 (s, 2H), 3.62- 3.54 (m, 4H), 2.56-2.41 (m, 4H). |
| | | MS m/z(ESI): 281.19 |
| | | [M + H]⁺. |
| | | HPLC purity: 99.52%. |
| Compound 20 | 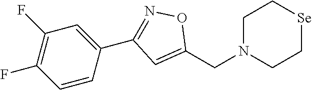 [SMILES: Fc1ccc(-c2cc(CN3CC[Se]CC3)on2)cc1F] | ¹H NMR (400 MHz, DMSO- d₆) δ 7.59-7.38 (m, 4H), 6.71 (s, 1H), 3.73 (s, 2H), 2.69-2.61 (m, 5H), 2.59- 2.51 (m, 5H). MS m/z(ESI): 345.13 |
| | | [M + H]⁺. |
| | | HPLC purity: 99.03%. |
| Compound 25 | 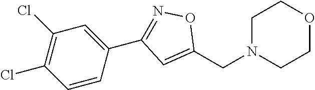 [SMILES: Clc1ccc(-c2cc(CN3CCOCC3)on2)cc1Cl] | ¹H NMR (400 MHz, DMSO- d₆) δ 7.74 (d, J = 2.2 Hz, 1H), 7.69-7.61 (m, 1H), 7.46 (d, J = 8.4 Hz, 1H), 6.75 (s, 1H), 3.93 (s, 2H), 3.62-3.54 (m, 4H), 2.56- |
| | | 2.41 (m, 5H). |
| | | MS m/z(ESI): 313.16 |
| | | [M + H]⁺. |
| | | HPLC purity: 98.73%. |
| Compound 26 | 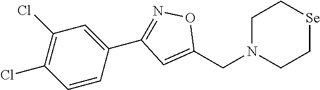 [SMILES: Clc1ccc(-c2cc(CN3CC[Se]CC3)on2)cc1Cl] | ¹H NMR (400 MHz, DMSO- d₆) δ 7.72 (d, J = 2.3 Hz, 1H), 7.68-7.60 (m, 1H), 7.42 (d, J = 8.3 Hz, 1H), 6.76 (s, 1H), 3.73 (s, 2H), 2.69-2.61 (m, 4H), 2.59- |
| | | 2.51 (m, 4H). |
| | | MS m/z(ESI): 377.30 |
| | | [M + H]⁺. |
| | | HPLC purity: 97.51%. |
| Compound 27 | 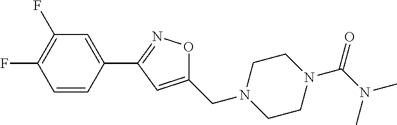 [SMILES: CN(C)C(=O)N1CCN(Cc2cc(-c3ccc(F)c(F)c3)no2)CC1] | ¹H NMR (400 MHz, DMSO- d₆) δ 7.62-7.49 (m, 2H), 7.43-7.33 (m, 1H), 6.79 (s, 1H), 3.92 (s, 2H), 3.52- 3.41 (m, 4H), 2.95 (s, 5H), 2.69-2.59 (m, 2H), 2.56- 2.45 (m, 2H). MS m/z(ESI): 351.30 |
| | | [M + H]⁺. |
| | | HPLC purity: 97.63%. |
| Compound 28 | 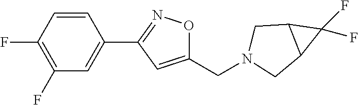 [SMILES: Fc1ccc(-c2cc(CN3CC4C(C3)C4(F)F)on2)cc1F] | ¹H NMR (400 MHz, DMSO- d₆) δ 7.61-7.50 (m, 2H), 7.43-7.33 (m, 1H), 6.72 (s, 1H), 3.84 (s, 2H), 3.15- 3.06 (m, 2H), 2.53-2.33 (m, 4H). MS m/z(ESI): 313.15 |
| | | [M + H]⁺. |
| | | HPLC purity: 98.07%. |
| Compound 29 | 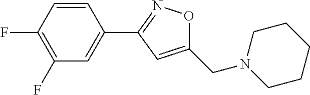 [SMILES: Fc1ccc(-c2cc(CN3CCCCC3)on2)cc1F] | ¹H NMR (400 MHz, DMSO- d₆) δ 7.61-7.52 (m, 2H), 7.43-7.32 (m, 1H), 6.71 (s, 1H), 3.91 (s, 2H), 2.60- 2.46 (m, 4H), 1.62-1.51 (m, 4H), 1.46-1.34 (m, 2H). |
| | | MS m/z(ESI): 279.23 |
| | | [M + H]⁺. |
| | | HPLC purity: 97.35%. |
| Compound 30 | 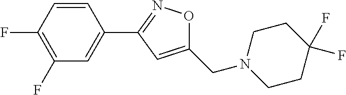 [SMILES: Fc1ccc(-c2cc(CN3CCC(F)(F)CC3)on2)cc1F] | ¹H NMR (400 MHz, DMSO- d₆) δ 7.62-7.47 (m, 2H), 7.43-7.33 (m, 1H), 6.98 (s, 1H), 3.91 (s, 2H), 2.44 (t, J = 5.8 Hz, 4H), 1.91- 1.83 (m, 1H), 1.83-1.73 (m, 3H). |
| | | MS m/z(ESI): 315.21 |
| | | [M + H]⁺. |
| | | HPLC purity: 98.54%. |
| Compound 31 | 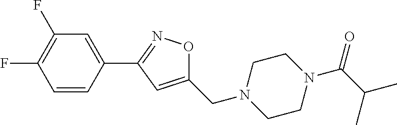 [SMILES: CC(C)C(=O)N1CCN(Cc2cc(-c3ccc(F)c(F)c3)no2)CC1] | ¹H NMR (400 MHz, DMSO- d₆) δ 7.63-7.47 (m, 2H), 7.43-7.33 (m, 1H), 7.00 (s, 1H), 3.91 (s, 2H), 3.40 (t, J = 5.2 Hz, 4H), 2.97- 2.85 (m, 1H), 2.55 (t, J = 5.2 Hz, 4H), 1.00 (d, J = 6.8 Hz, 6H). |
| | | MS m/z(ESI): 350.28 |
| | | [M + H]⁺. |
| | | HPLC purity: 96.76%. |
| Compound 32 | 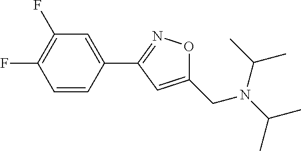 [SMILES: CC(C)N(Cc1cc(-c2ccc(F)c(F)c2)no1)C(C)C] | ¹H NMR (400 MHz, DMSO- d₆) δ 7.62-7.56 (m, 1H), 7.56-7.47 (m, 1H), 7.43- 7.33 (m, 1H), 6.98 (s, 1H), 3.91 (s, 2H), 3.11-2.96 (m, 2H), 1.07 (d, J = 6.8 Hz, 12H). MS m/z(ESI): 295.23 [M + H]⁺. HPLC purity: 98.33%. |
| Compound 34 | 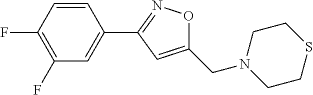 [SMILES: Fc1ccc(-c2cc(CN3CCSCC3)on2)cc1F] | ¹H NMR (400 MHz, DMSO- d₆) δ 7.62-7.47 (m, 2H), 7.43-7.33 (m, 1H), 6.98 (s, 1H), 3.91 (s, 2H), 2.97 (t, J = 5.3 Hz, 4H), 2.73 (t, J = 5.3 Hz, 4H). MS m/z(ESI): 297.17 |
| | | [M + H]⁺. |
| | | HPLC purity: 98.42%. |
| Compound 35 | 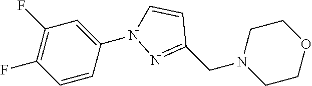 [SMILES: Fc1ccc(-n2ccc(CN3CCOCC3)n2)cc1F] | ¹H NMR (400 MHz, DMSO- d₆) δ 7.85-7.72 (m, 2H), 7.54-7.45 (m, 1H), 7.15- 7.05 (m, 1H), 6.49 (d, J = 7.4 Hz, 1H), 3.57 (dd, J = 8.4, 4.0 Hz, 6H), 2.41 (t, J = |
| | | 4.4 Hz, 4H). |
| | | MS m/z(ESI): 280.25 |
| | | [M + H]⁺. |
| | | HPLC purity: 97.52%. |
| Compound 36 | 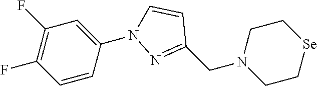 [SMILES: Fc1ccc(-n2ccc(CN3CC[Se]CC3)n2)cc1F] | ¹H NMR (400 MHz, DMSO- d₆) δ 7.95-7.84 (m, 2H), 7.33-7.25 (m, 1H), 7.22- 7.12 (m, 1H), 6.60 (d, J = 7.5 Hz, 1H), 3.55 (s, 2H), 2.40 (t, J = 7.1 Hz, 4H), |
| | | 1.40 (t, J = 7.1 Hz, 4H). |
| | | MS m/z(ESI): 344.19 |
| | | [M + H]⁺. |
| | | HPLC purity: 96.81%. |
| Compound 38 | 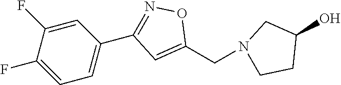 [SMILES: O[C@H]1CCN(Cc2cc(-c3ccc(F)c(F)c3)no2)C1] | ¹H NMR (400 MHz, DMSO- d₆) δ 7.63-7.54 (m, 1H), 7.54-7.47 (m, 1H), 7.43- 7.33 (m, 1H), 6.97 (d, J = 1.9 Hz, 1H), 5.02 (d, J = 5.0 Hz, 1H), 4.27 (d, J = 12.4 |
| | | Hz, 1H), 3.87-3.73 (m, |
| | | 1H), 3.49-3.36 (m, 2H), |
| | | 2.99 (dd, J = 9.5, 7.0 Hz, |
| | | 1H), 2.87-2.76 (m, 1H), |
| | | 2.12 (dd, J = 9.4, 7.0 Hz, |
| | | 1H), 1.81-1.68 (m, 1H), |
| | | 1.66-1.52 (m, 1H). |
| | | MS m/z(ESI): 281.20 |
| | | [M + H]⁺. |
| | | HPLC purity: 98.15%. |
| Compound 39 | 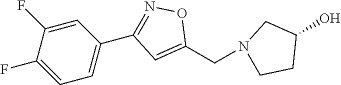 [SMILES: O[C@@H]1CCN(Cc2cc(-c3ccc(F)c(F)c3)no2)C1] | ¹H NMR (400 MHz, DMSO- d₆) δ 7.63-7.47 (m, 2H), 7.43-7.33 (m, 1H), 6.99 (d, J = 2.2 Hz, 1H), 5.02 (d, J = 5.0 Hz, 1H), 4.26 (d, J = 12.3 Hz, 1H), 3.87-3.73 |
| | | (m, 1H), 3.50-3.36 (m, |
| | | 2H), 3.29 (dd, J = 9.5, 7.0 |
| | | Hz, 1H), 2.86-2.74 (m, |
| | | 2H), 1.90-1.76 (m, 1H), |
| | | 1.65-1.51 (m, 1H). |
| | | MS m/z(ESI): 281.20 |
| | | [M + H]⁺. |
| | | HPLC purity: 98.83%. |
| Compound 40 | 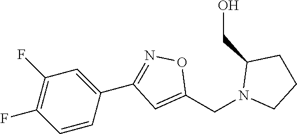 [SMILES: OC[C@H]1CCCN1Cc1cc(-c2ccc(F)c(F)c2)no1] | ¹H NMR (400 MHz, DMSO- d₆) δ 7.63-7.56 (m, 1H), 7.56-7.47 (m, 1H), 7.43- 7.33 (m, 1H), 6.99 (d, J = 1.8 Hz, 1H), 5.08 (t, J = 5.0 Hz, 1H), 4.51 (d, J = 12.3 Hz, 1H), 3.59-3.40 (m, 2H), 3.32 (dd, J = 12.5, 1.9 Hz, 1H), 3.16-3.02 (m, |
| | | 1H), 2.39-2.27 (m, 1H), |
| | | 2.10-2.00 (m, 1H), 1.95- |
| | | 1.81 (m, 1H), 1.67-1.49 |
| | | (m, 2H), 1.44-1.28 (m, |
| | | 1H). |
| | | MS m/z(ESI): 295.23 |
| | | [M + H]⁺. |
| | | HPLC purity: 98.15%. |
| Compound 41 | 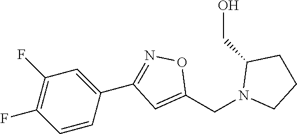 [SMILES: OC[C@@H]1CCCN1Cc1cc(-c2ccc(F)c(F)c2)no1] | ¹H NMR (400 MHz, DMSO- d₆) δ 7.63-7.56 (m, 1H), 7.56-7.47 (m, 1H), 7.43- 7.33 (m, 1H), 6.98 (d, J = 2.2 Hz, 1H), 4.57 (t, J = 5.5 Hz, 1H), 4.14 (d, J = 12.4 Hz, 1H), 3.62-3.51 (m, 2H), 3.37-3.26 (m, 1H), 3.07-2.97 (m, 1H), 2.27- |
| | | 2.15 (m, 1H), 2.12-2.00 |
| | | (m, 1H), 1.93-1.57 (m, |
| | | 3H), 1.40-1.25 (m, 1H). |
| | | MS m/z(ESI): 295.23 |
| | | [M + H]⁺. |
| | | HPLC purity: 98.56%. |
| Compound 42 | 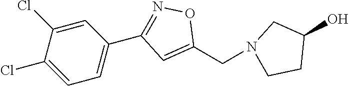 [SMILES: O[C@H]1CCN(Cc2cc(-c3ccc(Cl)c(Cl)c3)no2)C1] | ¹H NMR (400 MHz, DMSO- d₆) δ 7.82 (d, J = 2.1 Hz, 1H), 7.64 (dd, J = 7.5, 2.0 Hz, 1H), 7.39 (d, J = 7.5 Hz, 1H), 6.97 (d, J = 1.9 Hz, 1H), 5.02 (d, J = 5.0 Hz, |
| | | 1H), 4.26 (d, J = 12.5 Hz, |
| | | 1H), 3.87-3.73 (m, 1H), |
| | | 3.48-3.36 (m, 2H), 2.99 |
| | | (dd, J = 9.5, 6.9 Hz, 1H), |
| | | 2.87-2.76 (m, 1H), 2.12 |
| | | (dd, J = 9.4, 7.0 Hz, 1H), |
| | | 1.81-1.67 (m, 1H), 1.66- |
| | | 1.52 (m, 1H). |
| | | MS m/z(ESI): 313.17 |
| | | [M + H]⁺. |
| | | HPLC purity: 98.23%. |
| Compound 43 | 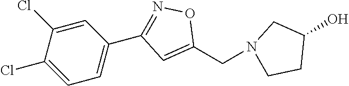 [SMILES: O[C@@H]1CCN(Cc2cc(-c3ccc(Cl)c(Cl)c3)no2)C1] | ¹H NMR (400 MHz, DMSO- d₆) δ 7.82 (d, J = 2.1 Hz, 1H), 7.64 (dd, J = 7.5, 2.1 Hz, 1H), 7.39 (d, J = 7.5 Hz, 1H), 6.98 (d, J = 2.1 Hz, 1H), 5.02 (d, J = 5.0 Hz, |
| | | 1H), 4.25 (d, J = 12.5 Hz, |
| | | 1H), 3.87-3.73 (m, 1H), |
| | | 3.50-3.37 (m, 2H), 3.29 |
| | | (dd, J = 9.4, 7.0 Hz, 1H), |
| | | 2.86-2.74 (m, 2H), 1.90- |
| | | 1.76 (m, 1H), 1.65-1.51 |
| | | (m, 1H). |
| | | MS m/z(ESI): 313.17 |
| | | [M + H]⁺. |
| | | HPLC purity: 98.78%. |
| Compound 44 | 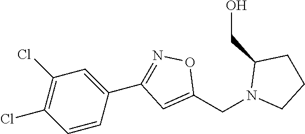 [SMILES: OC[C@H]1CCCN1Cc1cc(-c2ccc(Cl)c(Cl)c2)no1] | ¹H NMR (400 MHz, DMSO- d₆) δ 7.93 (d, J = 2.1 Hz, 1H), 7.76 (dd, J = 7.5, 2.0 Hz, 1H), 7.43 (d, J = 7.5 Hz, 1H), 7.05 (d, J = 2.0 Hz, 1H), 5.24 (t, J = 5.0 Hz, 1H), 4.32 (d, J = 12.4 Hz, 1H), 3.60-3.50 (m, 2H), 3.39 (dd, J = 12.3, 1.9 Hz, |
| | | 1H), 3.21-3.11 (m, 1H), |
| | | 2.47-2.37 (m, 1H), 2.10- |
| | | 2.00 (m, 1H), 1.95-1.85 |
| | | (m, 1H), 1.62-1.52 (m, |
| | | 2H), 1.42-1.32 (m, 1H). |
| | | MS m/z(ESI): 327.22 |
| | | [M + H]⁺. |
| | | HPLC purity: 98.43%. |
| Compound 45 | 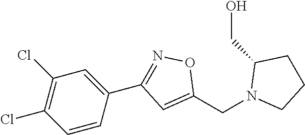 [SMILES: OC[C@@H]1CCCN1Cc1cc(-c2ccc(Cl)c(Cl)c2)no1] | ¹H NMR (400 MHz, DMSO- d₆) δ 7.96 (d, J = 2.0 Hz, 1H), 7.85 (dd, J = 7.5, 2.0 Hz, 1H), 7.47 (d, J = 7.5 Hz, 1H), 6.95 (d, J = 2.1 Hz, 1H), 4.80 (t, J = 5.5 Hz, 1H), 4.10 (d, J = 12.4 Hz, 1H), 3.70-3.60 (m, 2H), 3.35-3.25 (m, 1H), 3.08- |
| | | 2.98 (m, 1H), 2.28-2.18 |
| | | (m, 1H), 2.13-2.03(m, |
| | | 1H), 1.63-1.53 (m, 3H), |
| | | 1.31-1.21 (m, 1H). |
| | | MS m/z(ESI): 327.22 |
| | | [M + H]⁺. |
| | | HPLC purity: 98.11%. |
| Compound 46 | 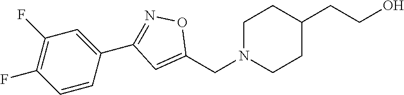 [SMILES: OCCC1CCN(Cc2cc(-c3ccc(F)c(F)c3)no2)CC1] | ¹H NMR (400 MHz, DMSO- d₆) δ 7.92-7.82 (m, 1H), 7.74-7.64 (m, 1H), 7.40- 7.30 (m, 1H), 6.90 (s, 1H), 4.53 (t, J = 5.0 Hz, 1H), 3.97 (s, 2H), 3.55-3.45 |
| | | (m, 2H), 2.96-2.86 (m, |
| | | 2H), 2.00-1.90 (m, 2H), |
| | | 1.95-1.85 (m, 2H), 1.66- |
| | | 1.56 (m, 2H), 1.42-1.32 |
| | | (m, 2H), 1.20-1.10 (m, |
| | | 1H). |
| | | MS m/z(ESI): 323.27 |
| | | [M + H]⁺. |
| | | HPLC purity: 98.24%. |
| Compound 47 | 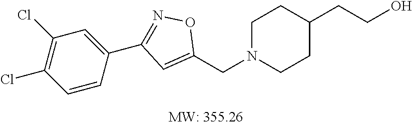 [SMILES: OCCC1CCN(Cc2cc(-c3ccc(Cl)c(Cl)c3)no2)CC1] | ¹H NMR (400 MHz, DMSO- d₆) δ 8.05. (d, J = 2.0 Hz, 1H), 7.84 (dd, J = 7.5, 2.0 Hz, 1H), 7.52 (d, J = 7.5 Hz, 1H), 7.06 (s, 1H), 4.47 (t, J = 5.0 Hz, 1H), 3.85 (s, 2H), 3.60-3.50 (m, 2H), 2.80- 2.70 (m, 2H), 2.00-1.90 |
| | | (m, 2H), 1.85-1.75 (m, |
| | | 2H), 1.68-1.58 (m, 2H), |
| | | 1.48-1.38 (m, 2H), 1.32- |
| | | 1.22 (m, 1H). |
| | | MS m/z(ESI): 355.23 |
| | | [M + H]⁺. |
| | | HPLC purity: 98.67%. |
| Compound 48 | 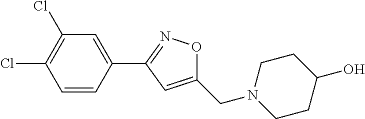 [SMILES: OC1CCN(Cc2cc(-c3ccc(Cl)c(Cl)c3)no2)CC1] | ¹H NMR (400 MHz, DMSO- d₆) δ 7.97 (d, J = 2.1 Hz, 1H), 7.71 (dd, J = 7.5, 2.1 Hz, 1H), 7.32 (d, J = 7.5 Hz, 1H), 6.87 (s, 1H), 3.85 (s, 2H), 3.62-3.52 (m, 1H), 3.16-3.06 (m, 3H), 2.20- 2.10 (m, 2H), 1.75-1.65 |
| | | (m, 2H), 1.55-1.45 (m, |
| | | 2H). |
| | | MS m/z(ESI): 327.22 |
| | | [M + H]⁺. |
| | | HPLC purity: 98.85%. |
| Compound 49 | 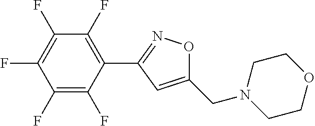 [SMILES: Fc1c(F)c(F)c(-c2cc(CN3CCOCC3)on2)c(F)c1F] | ¹H NMR (400 MHz, DMSO- d₆) δ 6.95 (s, 1H), 3.86 (s, 2H), 3.66 (t, J = 4.7 Hz, 4H), 2.35 (t, J = 4.7 Hz, 4H). MS m/z(ESI): 335.18 [M + H]⁺. HPLC purity: 97.13%. |
| Compound 50 | 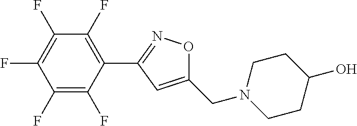 [SMILES: OC1CCN(Cc2cc(-c3c(F)c(F)c(F)c(F)c3F)no2)CC1] | ¹H NMR (400 MHz, DMSO- d₆) δ 6.85 (s, 1H), 3.87 (s, 2H), 3.50-3.40 (m, 1H), 3.10-3.00 (m, 3H), 2.30- 2.20 (m, 2H), 1.80-1.70 (m, 2H), 1.40-1.30 (m, 2H). MS m/z(ESI): 349.24 |
| | | [M + H]⁺. |
| | | HPLC purity: 96.03%. |

### Example 8: Preparation of Compound 65

### Preparation of Compound 65a

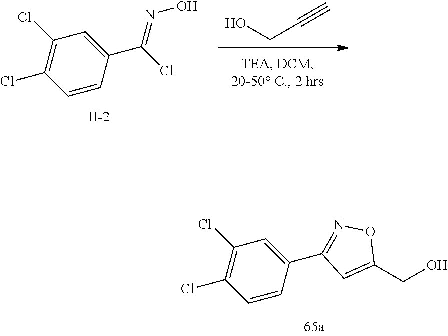

Intermediate II-2 (1.00 g, 4.45 mmol, 1.00 eq) was dissolved in DCM (10.0 mL), and triethylamine (TEA, 676 mg, 6.68 mmol, 930 μL, 1.50 eq) was added. At 20° C., 2-propyn-1-ol (299 mg, 5.35 mmol, 315 μL, 1.20 eq) was added. The reaction was stirred at 50° C. for 2 hours, and LCMS detected the target molecular weight. At 10° C., NaOH (aq, 2.00 mL) was added to the reaction mixture and stirred for 0.5 hours. The reaction mixture was diluted with 3.00 mL water, extracted with 3.00 mL DCM, and the organic phase was washed with 6.00 mL of saturated brine (2.00 mL×3). The mixture was dried over Na₂SO₄, filtered, and concentrated under reduced pressure to obtain a concentrate. Column chromatography purification (SiO₂, PE/EA=1/1, R_{f}=0.45) yielded a grey-white solid compound 65a (170 mg, 615 μmol, yield 13.8%, purity 88.3%).

LCMS: m/z=244.0, M+H⁺

### Preparation of Compound 65b

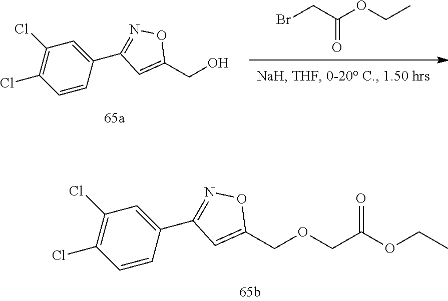

Compound 65a (170 mg, 696 μmol, 1.00 eq) was dissolved in THE (2.00 mL), and at 0° C., NaH (30.6 mg, 766 μmol, 60.0% purity, 1.10 eq) was added. The mixture was stirred for 0.5 hours, then ethyl bromoacetate (127 mg, 766 μmol, 84.7 μL, 1.10 eq) was added. The reaction was stirred at 20° C. for 1 hour, and LCMS showed the formation of 72.6% of the target product (m/z=330.1, M+H⁺). At 10° C., the reaction was quenched with NH₄Cl (2.00 mL). The mixture was stirred at 10° C. for 0.5 hours, diluted with 2 mL water, and extracted with 2 mL of ethyl acetate. The organic phase was washed with 6.00 mL of saturated brine (2.00 mL×3), dried over Na₂SO₄, filtered, and concentrated under reduced pressure to obtain a concentrate. Purification by preparative thin-layer chromatography (SiO₂, PE/EA=2/1, R_{f}=0.45) yielded a yellow solid compound 65b (170 mg, 511 μmol, yield 73.4%, purity 99.3%).

LCMS: m/z=330.1, M+H⁺

### Preparation of Compound 65c

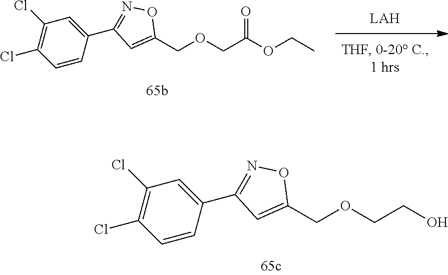 [SMILES: CCOC(=O)COCc1cc(-c2ccc(Cl)c(Cl)c2)no1 ; OCCOCc1cc(-c2ccc(Cl)c(Cl)c2)no1]

Compound 65b (100 mg, 302 μmol, 1.00 eq) was dissolved in THE (1.00 mL), and at 0° C., lithium aluminium hydride (LAH, 2.5 M, 157 μL, 1.30 eq) was added. The reaction was stirred at 20° C. for 1 hour, and LCMS showed the formation of 93.7% of the target product (m/z=288.3, M+H⁺). At 0° C., the reaction was quenched with H₂O (0.05 mL), followed by dropwise addition of NaOH (0.05 mL), and then H₂O (0.15 mL) was added. The reaction mixture was filtered and concentrated under reduced pressure to obtain a yellow oily compound 65c (94.0 mg, 286 μmol, yield 94.4%, purity 87.7%).

LCMS: m/z=288.3, M+H⁺

### Preparation of Compound 65d

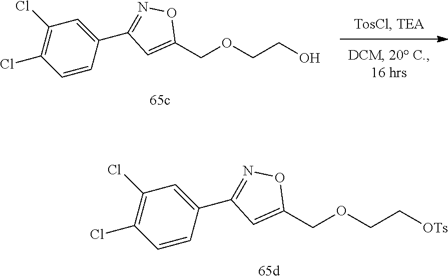 [SMILES: OCCOCc1cc(-c2ccc(Cl)c(Cl)c2)no1 ; Cc1ccc(S(=O)(=O)OCCOCc2cc(-c3ccc(Cl)c(Cl)c3)no2)cc1]

Compound 65c (50.0 mg, 173 μmol, 1.00 eq) was dissolved in DCM (0.50 mL), and triethylamine (TEA, 52.6 mg, 520 μmol, 72.4 μL, 3.00 eq) and tosyl chloride (TosCl, 36.3 mg, 190 μmol, 1.10 eq) were added. The reaction was stirred at 20° C. for 16 hours, and LCMS showed the formation of the target product (m/z=442.0, M+H⁺). The reaction mixture was diluted with 3.00 mL water and extracted with 3.00 mL DCM. The organic phase was washed with 6.00 mL of saturated brine (2.00 mL×3), dried over Na₂SO₄, filtered, and concentrated under reduced pressure to obtain a concentrate. Purification by preparative thin-layer chromatography (SiO₂, PE/EA=1/1, R_{f}=0.58) yielded a yellow oily compound 65d (40.0 mg, 77.9 μmol, yield 44.9%, purity 86.2%).

LCMS: m/z=442.0, M+H⁺

### Preparation of Compound 65

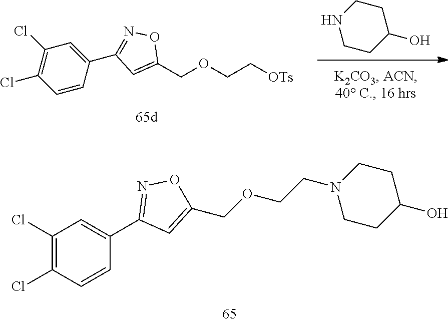 [SMILES: Cc1ccc(S(=O)(=O)OCCOCc2cc(-c3ccc(Cl)c(Cl)c3)no2)cc1 ; OC1CCNCC1 ; OC1CCN(CCOCc2cc(-c3ccc(Cl)c(Cl)c3)no2)CC1]

Compound 65d (10.0 mg, 22.6 μmol, 1.00 eq) was dissolved in ACN (0.50 mL), and K₂CO₃ (9.37 mg, 67.8 μmol, 3.00 eq) and 4-hydroxypiperidine (2.52 mg, 24.8 μmol, 1.10 eq) were added. The mixture was stirred at 40° C. for 16 hours. LC-MS showed the formation of 67.0% of the target product (m/z=371.1, M+H⁺). The reaction mixture was filtered and concentrated under reduced pressure to obtain a concentrate.

Purification (FA condition, column: Phenomenex Luna C18 150×25 mm×10 μm; mobile phase: [water (FA)-ACN]; B %: 18%-48%, 10 min) yielded a yellow gel-like compound 65 (2.26 mg, 5.75 μmol, yield 25.4%, purity 94.5%).

LCMS: m/z=371.1, M+H⁺

¹H NMR: 400 MHz, CDCl₃ b 7.91 (d, J=2.0 Hz, 1H), 7.64-7.67 (m, 1H), 7.54 (d, J=8.4 Hz, 1H), 6.59 (s, 1H), 4.69 (s, 2H), 3.82-3.85 (m, 3H), 3.02-3.04 (m, 2H), 2.88 (t, J=5.2 Hz, 2H), 2.24 (s, 2H), 2.03-2.06 (m, 2H), 1.73-1.76 (m, 2H).

### Example 9: Preparation of Compound 81

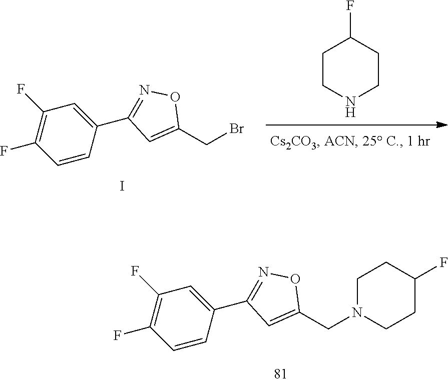

Intermediate I (200 mg, 444 μmol, 1.00 eq) was dissolved in ACN (3.00 mL), and 4-fluoropiperidine (75.2 mg, 729 μmol, 1.00 eq) and Cs₂CO₃ (475 mg, 1.46 mmol, 2.00 eq) were added. The mixture was stirred at 25° C. for 1 hour, and LCMS showed the complete reaction of Intermediate I and detected a new main peak. The mixture was filtered through diatomaceous earth, the filter cake was washed with ACN, and the combined organic phases were concentrated under reduced pressure to obtain a concentrate. Purification (column: Waters Xbridge 150×25 mm×5 μm; mobile phase: [water (FA)-ACN]; gradient: 8%-38% B over 12 min) yielded a white solid compound 81 (203 mg, 592 μmol, yield 81.1%, purity 99.9%, FA).

LCMS: m/z=297.4, M+H⁺

¹H NMR: (400 MHz, CDCl₃) δ 7.65-7.66 (m, 1H), 7.52-7.55 (m, 1H), 7.24-7.27 (m, 1H), 6.47 (s, 1H), 4.64-4.80 (m, 1H), 3.76 (s, 2H), 2.68-2.71 (m, 2H), 2.55-2.59 (m, 2H), 1.89-1.97 (m, 4H).

The preparation of compounds listed below is carried out with reference to Example 9

| Name | Structure | Data |
|---|---|---|
| Compound 79 | 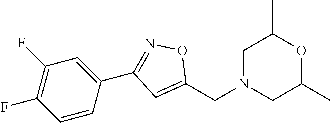 [SMILES: CC1CN(Cc2cc(-c3ccc(F)c(F)c3)no2)CC(C)O1] | ¹H NMR (400 MHz, MeOD) δ 7.65-7.66 (m, 1H), 7.53- 7.55 (m, 1H), 7.24-7.27 (m, 1H), 6.47(s, 1H), 3.69-3.76 (m, 4H), 2.78 (d, J = 10.4 Hz, 2H), 1.92 (t, J = 10.8 Hz, 2H), 1.17 (d, J = 6.4 Hz, 1H). |
| | | MS m/z(ESI): 309.7 [M + H]⁺. |
| | | HPLC purity: 99.7%. |
| Compound 80 | 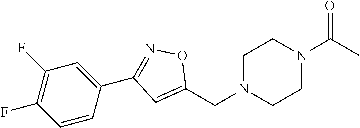 [SMILES: CC(=O)N1CCN(Cc2cc(-c3ccc(F)c(F)c3)no2)CC1] | ¹H NMR (400 MHz, MeOD) δ 7.64-7.66 (m, 1H), 7.53- 7.54 (m, 1H), 7.24-7.27 (m, 1H), 6.46 (s, 1H), 3.77 (s, 2H), 3.65-3.68 (m, 2H), 3.49-3.52 (m, 2H), 2.52- 2.58 (m, 4H), 2.09 (s, 3H). MS m/z(ESI): 322.3 [M + H]⁺. |
| | | HPLC purity: 99.8%. |
| Compound 82 | 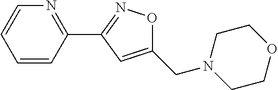 [SMILES: c1ccc(-c2cc(CN3CCOCC3)on2)nc1] | ¹H NMR (400 MHz, CDCl₃) δ 8.68 (d, J = 4.80 Hz, 1H), 8.08 (d, J = 4.00 Hz, 1H), 7.75-7.85 (m, 1H), 7.30- 7.40 (m, 1H), 6.88 (s, 1H), 3.60-3.90 (m, 6H), 2.61 (s, |
| | | 1H). |
| | | MS m/z(ESI): 246.1 [M + H]⁺. |
| | | HPLC purity: 99.2%. |
| Compound 85 | 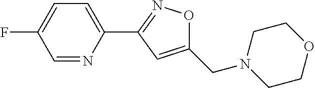 [SMILES: Fc1ccc(-c2cc(CN3CCOCC3)on2)nc1] | ¹H NMR (400 MHz, CDCl₃) δ 8.52 (s, 1H), 8.08-8.12 (m, 1H), 7.50-7.54 (m, 1H), 6.81 (s, 1H), 3.73 (t, J = 6.0 Hz, 6H), 2.57-2.59 (m, 4H) MS m/z(ESI): 264.3 [M + H]⁺. |
| | | HPLC purity: 99.6%. |
| Compound 86 | 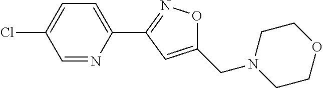 [SMILES: Clc1ccc(-c2cc(CN3CCOCC3)on2)nc1] | ¹H NMR (400 MHz, MeOD) δ 8.70 (d, J = 2.4 Hz, 1H), 8.11 (d, J = 8.4 Hz, 1H), 8.00-8.03 (m, 1H), 7.38 (s, 1H), 4.77 (s, 2H), 4.08 (s, 2H), 3.85 (s, 2H), 3.35 (s, |
| | | 2H), 3.31 (s, 2H). |
| | | MS m/z(ESI): 280.3 [M + H]⁺. |
| | | HPLC purity: 98.8%. |
| Compound 88 | 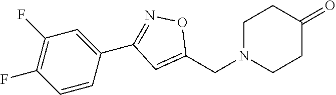 [SMILES: O=C1CCN(Cc2cc(-c3ccc(F)c(F)c3)no2)CC1] | ¹H NMR (400 MHz, CDCl₃) δ 7.66-7.69 (m, 1H), 7.54- 7.56 (m, 1H), 7.26-7.28 (m, 1H), 6.51 (s, 1H), 3.89 (s, 2H), 2.90 (t, J = 5.6 Hz, 4H), 2.52 (t, J = 6.0 Hz, |
| | | 4H). |
| | | MS m/z(ESI): 293.1, |
| | | [M + H]⁺. |
| | | HPLC purity: 98.5%. |
| Compound 90 | 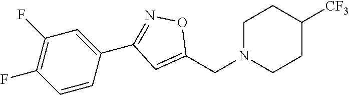 [SMILES: Fc1ccc(-c2cc(CN3CCC(C(F)(F)F)CC3)on2)cc1F] | ¹H NMR (400 MHz, CDCl₃) δ 7.76-7.81 (m, 1H), 7.66- 7.67 (m, 1H), 7.38-7.41 (m, 1H), 6.81 (s, 1H), 3.80 (s, 2H), 3.04-3.07 (m, 2H), 2.16-2.23 (m, 3H), 1.87- |
| | | 1.90 (m, 2H), 1.62-1.66 (m, |
| | | 2H). |
| | | MS m/z(ESI): 347.4 [M + H]⁺. |
| | | HPLC purity: 98.8%. |
| Compound 93 | 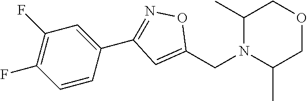 [SMILES: CC1COCC(C)N1Cc1cc(-c2ccc(F)c(F)c2)no1] | ¹H NMR (400 MHz, CDCl₃) δ 7.63-7.68 (m, 1H), 7.53- 7.55 (m, 1H), 7.21-7.26 (m, 1H), 6.48 (s, 1H), 4.00-4.03 (m, 1H), 3.68-3.75 (m, 3H), 3.40-3.44 (m, 2H), 2.91- 2.95 (m, 2H), 1.07 (d, J = |
| | | 6.4 Hz, 6H). |
| | | MS m/z(ESI): 309.4 [M + H]⁺. |
| | | HPLC purity: 99.4%. |
| Compound 94 | 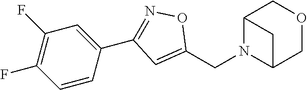 [SMILES: Fc1ccc(-c2cc(CN3C4COCC3C4)on2)cc1F] | ¹H NMR (400 MHz, CDCl₃) δ 7.66-7.69 (m, 1H), 7.54- 7.56 (m, 1H), 7.26-7.28 (m, 1H), 6.51 (s, 1H), 3.89 (s, 2H), 2.90 (t, J = 5.6 Hz, 4H), 2.52 (t, J = 6.0 Hz, |
| | | 4H). |
| | | MS m/z(ESI): 293.3 [M + H]⁺. |
| | | HPLC purity: 99.6%. |

### Example 10: Preparation of Compound 91

### Preparation of Compound 91a

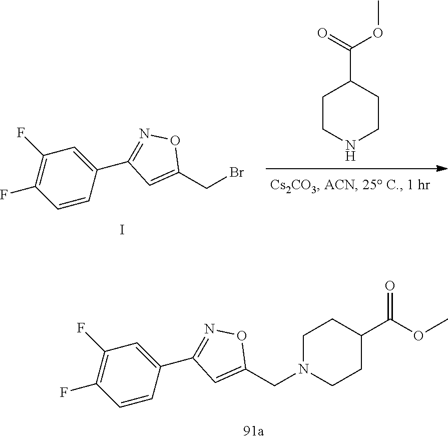

Intermediate I (400 mg, 1.46 mmol, 1.00 eq) was dissolved in ACN (4.00 mL), and Cs₂CO₃ (951 mg, 2.92 mmol, 2.00 eq) and 4-piperidinecarboxylate methyl ester (251 mg, 1.75 mmol, 1.20 eq) were added. The mixture was stirred at 25° C. for 1 hour, and LCMS showed the complete reaction of Intermediate I (m/z=337.3, M+H⁺) and the formation of the target product's main peak. At 20° C., the reaction was quenched by adding 4.00 mL of water, extracted with dichloromethane (DCM, 4.00 mL×3), and the combined organic phase was washed with saturated brine (4.00 mL×2), dried over Na₂SO₄, filtered, and concentrated to obtain a concentrate. The crude white solid compound 91a (500 mg, crude product) was used directly for the next step without further purification.

LCMS: m/z=337.3, M+H⁺

### Preparation of Compound 91

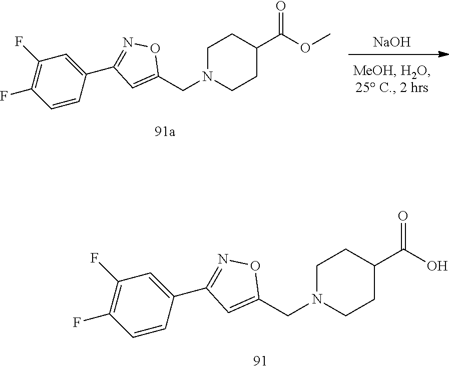 [SMILES: COC(=O)C1CCN(Cc2cc(-c3ccc(F)c(F)c3)no2)CC1 ; "NaOH" ; O=C(O)C1CCN(Cc2cc(-c3ccc(F)c(F)c3)no2)CC1]

Compound 91a (409 mg, 1.22 mmol, 1.00 eq) was dissolved in methanol (5.00 mL) and water (2.50 mL), and sodium hydroxide (146 mg, 3.65 mmol, 3.00 eq) was added. The mixture was stirred at 25° C. for 2 hours. LCMS showed that compound 91a (m/z=323.3, M+H⁺) reacted completely and the main peak of the target product was formed. At 20° C., the reaction was quenched by adding 3.00 mL of water, then extracted with DCM (3.00 mL×3), and the combined organic phase was washed with saturated brine (3.00 mL×2), dried over Na₂SO₄, filtered, and concentrated to obtain a concentrate. Purification (column: Phenomenex Luna C18 15025 mm 10 μm; mobile phase: [water (FA)-ACN]; B %: 7%-37%, 2 min) yielded a white solid compound 91 (136 mg, 406 μmol, yield 33.4%, purity 96.3%).

LCMS: m/z=323.3, M+H⁺

¹H NMR: (400 MHz, CDCl₃) δ 11.7 (s, 1H), 7.94-8.13 (m, 1H), 7.75-7.75 (m, 1H), 7.58-7.63 (m, 1H), 7.04 (s, 1H), 3.74 (s, 2H), 2.82 (d, J=10.4 Hz, 2H), 2.15-2.20 (m, 3H), 1.79-1.82 (m, 2H), 1.56-1.59 (m, 2H).

### Example 11: Preparation of Compound 87

### Preparation of Compound 87a

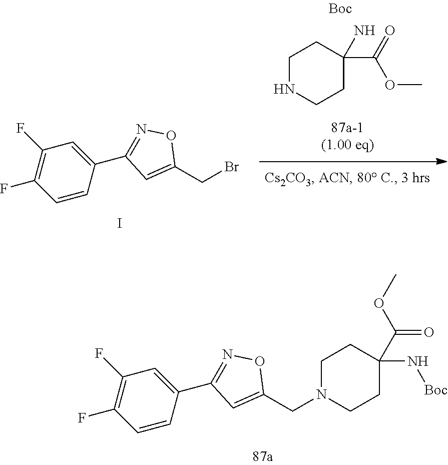

Compound I (300 mg, 1.09 mmol, 1.00 eq) and compound 87a-1 (283 mg, 1.09 mmol, 1.00 eq) were dissolved in ACN (3.00 mL), and Cs₂CO₃ (713 mg, 2.19 mmol, 2.00 eq) was added. The mixture was stirred at 80° C. for 3 hours, and LCMS showed that compound I reacted completely, with the formation of a new peak m/z (MS=452.7, M+H⁺). The reaction mixture was filtered and concentrated under reduced pressure to obtain a yellow gel-like substance compound 87a (500 mg, crude product).

LCMS: MS=452.7, M+H⁺

### Preparation of Compound 87b

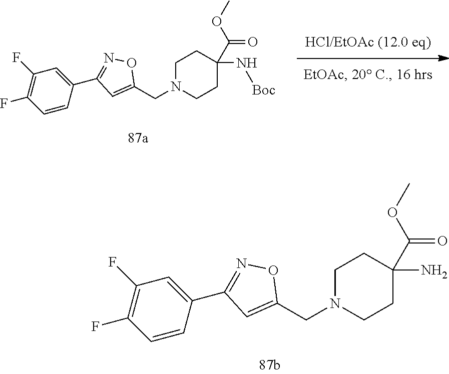 [SMILES: COC(=O)C1(NC(=O)OC(C)(C)C)CCN(Cc2cc(-c3ccc(F)c(F)c3)no2)CC1 ; COC(=O)C1(N)CCN(Cc2cc(-c3ccc(F)c(F)c3)no2)CC1]

Compound 87a (500 mg, 1.11 mmol, 1.00 eq) was dissolved in EtOAc (5.00 mL), and HCl in EtOAc (4 M, 3.32 mL, 12.0 eq) was added. The mixture was stirred at 20° C. for 16 hours. LCMS showed that compound 87a reacted completely, with the formation of a new target product peak m/z (MS=352.4, M+H⁺). Concentrating the reaction mixture under reduced pressure yielded a white solid compound 87b (450 mg, crude product, HCl).

LCMS: MS=352.4, M+H⁺

### Preparation of Compound 87

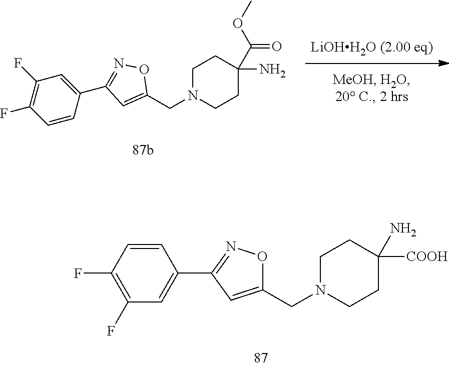 [SMILES: COC(=O)C1(N)CCN(Cc2cc(-c3ccc(F)c(F)c3)no2)CC1 ; NC1(C(=O)O)CCN(Cc2cc(-c3ccc(F)c(F)c3)no2)CC1]

Compound 87b (450 mg, 1.28 mmol, 1.00 eq) was dissolved in MeOH (3.60 mL) and H₂O (0.90 mL), and LiOH·H₂O (107 mg, 2.56 mmol, 2.00 eq) was added. The mixture was stirred at 20° C. for 2 hours, and LCMS showed that compound 87b reacted completely, with the formation of a new target product peak m/z (MS=338.3, M+H⁺). The reaction mixture was filtered and concentrated under reduced pressure to obtain a concentrate. Purification (column: Phenomenex Luna C18 200*40 mm*10 μm; mobile phase: [water (HCl)-ACN]; B %: 3%-33%, 10 min) yielded a white solid compound 87 (200 mg, 584 μmol, yield 45.6%, purity 98.5%).

LCMS: MS=338.3, M+H⁺

¹H NMR: (400 MHz, DMSO-d₆) δ 7.94-7.99 (m, 1H), 7.77 (t, J=4.8 Hz, 1H), 7.59-7.61 (m, 1H), 7.06 (s, 1H), 3.79 (s, 2H), 2.65 (s, 4H), 2.06-2.12 (m, 2H), 1.80-1.83 (m, 2H).

### Example 12: Preparation of Compound 51

### Preparation of Compound 51a

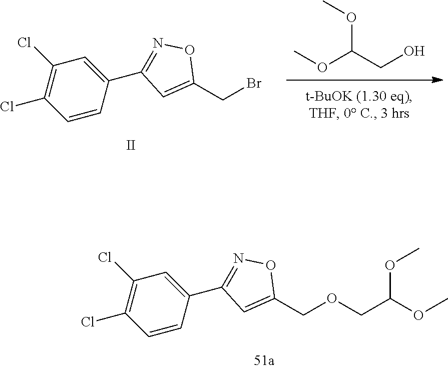

Intermediate II (0.10 g, 322 μmol, 98.8% purity, 1.00 eq) was dissolved in THF (2.00 mL), and 2,2-dimethoxyethanol (68.3 mg, 644 μmol, 2.00 eq) and t-BuOK (1.00 M, 966 μL, 3.00 eq) were added. The mixture was stirred at 0° C. for 3 hours, and TLC (PE:EA=3:1) monitored the complete reaction of compound II (R_{f}=0.65) and the formation of many new spots (R_{f}=0.35). At 20° C., the reaction was quenched by adding 4.00 mL H₂O, extracted with EtOAc (8.00 mL×3), and washed with saturated brine (10.0 mL×2). The combined organic phases were dried over Na₂SO₄, filtered, and concentrated under reduced pressure to obtain a concentrate. Purification (SiO₂, PE:EA=3:1) yielded a pale-yellow liquid compound 51a (56.0 mg, 164 μmol, yield 50.9%, purity 97.2%).

¹H NMR: 400 MHz, CDCl₃ δ 7.91 (d, J=2.0 Hz, 1H), 7.64-7.67 (m, 1H), 7.55 d, J=8.4 Hz, 1H), 6.58 (s, 1H), 4.73 (s, 2H), 4.54-4.57 (m, 1H), 3.64 (d, J=5.2 Hz, 1H), 3.41-3.45 (m, 6H).

### Preparation of Compound 51b

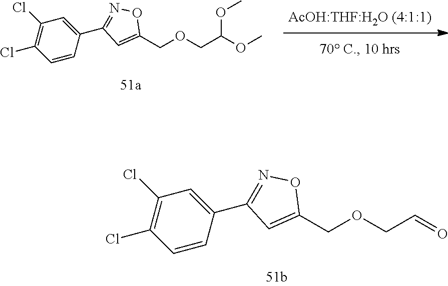 [SMILES: COC(COCc1cc(-c2ccc(Cl)c(Cl)c2)no1)OC ; O=CCOCc1cc(-c2ccc(Cl)c(Cl)c2)no1]

Compound 51a (0.05 g, 151 μmol, 1.00 eq) was dissolved in AcOH (0.40 mL), THF (0.10 mL), and water (0.10 mL). The mixture was stirred at 70° C. for 10 hours, and LCMS showed that compound 51a reacted completely, producing a new target peak (m/z=286.1, M+H⁺). The reaction mixture was concentrated under pressure to obtain a yellow oily compound 51b (0.05 g, crude product).

LCMS: m/z=286.1, M+H⁺

### Preparation of Compound 51

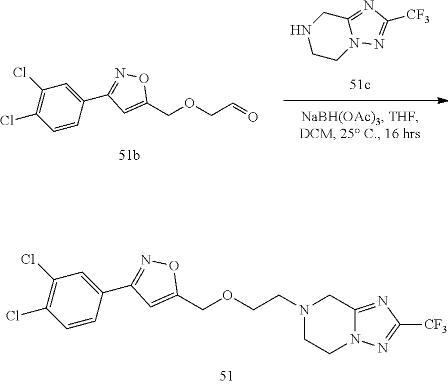 [SMILES: FC(F)(F)c1nc2n(n1)CCNC2 ; O=CCOCc1cc(-c2ccc(Cl)c(Cl)c2)no1 ; FC(F)(F)c1nc2n(n1)CCN(CCOCc1cc(-c3ccc(Cl)c(Cl)c3)no1)C2]

Compound 51b (0.04 g, 139 μmol, 1.00 eq) was dissolved in THE (0.50 mL) and DCM (0.50 mL), and compound 51c (32.2 mg, 167 μmol, 1.20 eq) and NaBH(OAc)₃ (59.3 mg, 279 μmol, 2.00 eq) were added. The mixture was stirred at 20° C. for 16 hours. LCMS showed that compound 51b reacted completely, with the formation of a new target peak (m/z=462.2, M+H⁺). The crude product was purified (column: Phenomenex Luna C18 150×25 mm×10 μm; mobile phase: [water (FA)-ACN]; B %: 57%-87%, 10 min) to obtain a yellow oily compound 51 (24.0 mg, 50.8 μmol, yield 36.4%, purity 97.9%).

LCMS: m/z=462.2, M+H⁺

¹H NMR: 400 MHz, CDCl₃ δ 7.91 (t, J=2.0 Hz, 1H), 7.64-7.66 (m, 1H), 7.55 (d, J=8.4 Hz, 1H), 6.56 (s, 1H), 4.69 (s, 2H), 4.28 (t, J=5.6 Hz, 2H), 3.96 (s, 2H), 3.81 (t, J=5.2 Hz, 2H), 3.16-3.15 (m, 2H), 2.94-2.96 (m, 2H).

The preparation of compounds listed below is carried out with reference to Example 12

| Name | Structure | Data |
|---|---|---|
| Compound 52 | 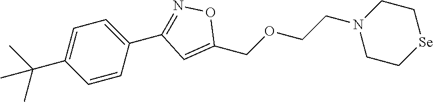 [SMILES: CC(C)(C)c1ccc(-c2cc(COCCN3CC[Se]CC3)on2)cc1] | ¹H NMR (400 MHz, CDCl₃) δ 7.73-7.76 (m, 2H), 7.48- 7.51 (m, 2H), 6.56 (s, 1H), 4.67 (s, 2H), 3.68-3.71 (m, 2H), 2.94-2.96 (m, 4H), 2.68-2.75 (m, 6H), 1.36 (s, 9H). |
| | | MS m/z(ESI): 409.2 [M + H]⁺. |
| Compound 53 | 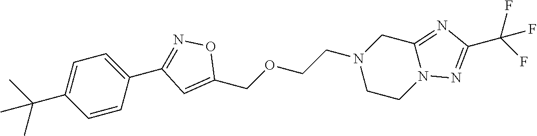 [SMILES: CC(C)(C)c1ccc(-c2cc(COCCN3CCn4nc(C(F)(F)F)nc4C3)on2)cc1] | ¹H NMR (400 MHz, CDCl₃) δ 7.74 (d, J = 8.4 Hz, 2H), 7.49 (d, J = 8.4 Hz, 2H), 6.56 (s, 1H), 4.69 (s, 2H), 4.26-4.28 (m, 2H), 3.94 (s, 2H), 3.78-3.81 (m, 2H), 3.13-3.16 (m, 2H), 2.92- 2.94 (m, 2H), 1.36 (s, 9H). MS m/z(ESI): 450.3 [M + H]⁺. |
| | | HPLC purity: 91.7%. |
| Compound 59 | 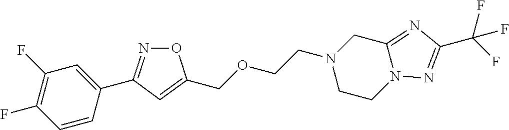 [SMILES: Fc1ccc(-c2cc(COCCN3CCn4nc(C(F)(F)F)nc4C3)on2)cc1F] | ¹H NMR (400 MHz, CDCl₃) δ 7.63-7.69 (m, 1H), 7.51- 7.56 (m, 1H), 7.26-7.30 (m, 1H), 6.53 (s, 1H), 4.69 (s, 2H), 4.27-4.29 (m, 2H), 3.95 (s, 2H), 3.79-3.82 (m, 2H), 3.14-3.17 (m, 2H), 2.93-2.96 (m, 2H). MS m/z(ESI): 430.0 [M + H]⁺. |
| | | HPLC purity: 96.5%. |
| Compound 76 | 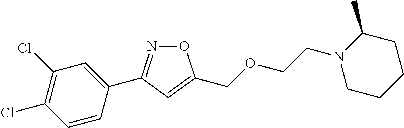 [SMILES: C[C@H]1CCCCN1CCOCc1cc(-c2ccc(Cl)c(Cl)c2)no1] | ¹H NMR (400 MHz, CDCl₃) δ 7.92 (d, J = 2.0 Hz, 1H), 7.64-7.69 (m, 1H), 7.54 (d, J = 8.4 Hz, 1H), 6.59 (s, 1H), 4.69 (s, 2H), 3.79- 3.82 (m, 2H), 3.03-3.11 (m, 2H), 2.46-2.75 (m, 3H), 1.69-1.74 (m, 4H), 1.35-1.49 (m, 2H), 1.19 (d, |
| | | J = 6.0 Hz, 3H) |
| | | MS m/z(ESI): 369.1 [M + H]⁺. |
| | | HPLC purity: 98.9%. |
| Compound 77 | 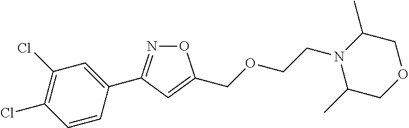 [SMILES: CC1COCC(C)N1CCOCc1cc(-c2ccc(Cl)c(Cl)c2)no1] | ¹H NMR (400 MHz, CDCl₃) δ 7.92 (d, J = 1.6 Hz, 1H), 7.64-7.66 (m, 1H), 7.55 (d, J = 8.4 Hz, 1H), 6.60 (s, 1H), 4.69 (s, 2H), 3.83- 4.01 (m, 4H), 3.61-3.65 (m, 2H), 3.36 (s, 3H), 2.88- 3.01 (m, 1H), 1.23-1.28 |
| | | (m, 6H). |
| | | MS m/z(ESI): 385.2 [M + H]⁺. |
| | | HPLC purity: 98.5%. |

### Example 13: Preparation of Compound 78

### Preparation of Compound 78b

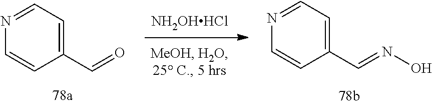 [SMILES: O=Cc1ccncc1 ; "NH2OH·HCl" ; O/N=C/c1ccncc1]

Compound 78a (1.00 g, 9.34 mmol, 877 μL, 1.00 eq) was dissolved in MeOH (5.00 mL) and H₂O (5.00 mL), and NH₂OH·HCl (778 mg, 11.2 mmol, 1.20 eq) was added. The mixture was stirred at 25° C. for 5 hours, and TLC (PE:EA=1:1, R_{f}=0.45) monitored the complete reaction of compound 78a and the formation of a new spot. The reaction mixture was filtered and concentrated under reduced pressure to obtain a white solid 78b (1.20 g, crude product).

¹H NMR: (400 MHz, DMSO-d₆) δ 10.3 (s, 1H), 8.87 (d, J=6.8 Hz, 2H), 8.41 (s, 1H), 8.12 (d, J=6.4 Hz, 2H).

### Preparation of Compound 78c

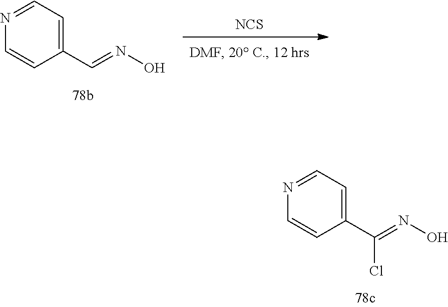 [SMILES: O/N=C/c1ccncc1 ; "NCS" ; O/N=C(\Cl)c1ccncc1]

Compound 78b (500 mg, 4.09 mmol, 1.00 eq) was dissolved in DMF (5.00 mL), and NCS (546 mg, 4.09 mmol, 1.00 eq) was added. The mixture was stirred at 20° C. for 12 hours, and TLC (PE:EA=1:1, R_{f}=0.55) monitored the complete reaction of compound 78b and the formation of a new spot. The reaction mixture was filtered and concentrated under reduced pressure to obtain a white solid compound 78c (700 mg, crude product).

¹H NMR: (400 MHz, DMSO-d₆) δ 11.0 (s, 1H), 8.85 (d, J=6.4 Hz, 2H), 8.11 (d, J=6.4 Hz, 2H).

### Preparation of Compound 78d

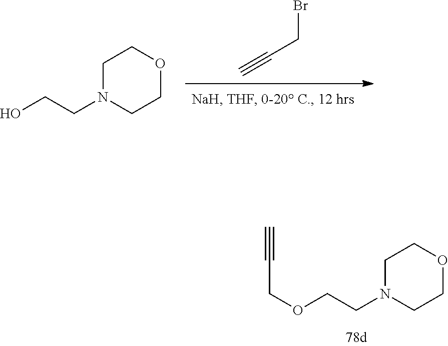

2-Morpholinoethanol (2.12 g, 16.1 mmol, 1.97 mL, 1.20 eq) was dissolved in THE (20.0 mL), and NaH (645 mg, 16.1 mmol, 60.0% content, 1.20 eq) was added dropwise at 0° C., stirring for 0.5 hours. At 20° C., 3-bromopropyne (2.00 g, 13.4 mmol, 1.45 mL, 1.00 eq) was added dropwise, stirring for 11.5 hours. LCMS showed the complete reaction of 2-morpholinoethanol and the formation of a new target product peak (m/z=170.1, M+H⁺). At 0° C., 10.0 mL H₂O was added to quench the reaction, and the mixture was extracted with EtOAc (10.0 mL×3), washed with saturated brine (10.0 mL×3), and the combined organic phases were dried over Na₂SO₄, filtered, and concentrated under reduced pressure to obtain a yellow liquid compound 78d (2.20 g, 13.0 mmol, yield 96.7%).

LCMS: m/z=170.1, M+H⁺

### Preparation of Compound 78

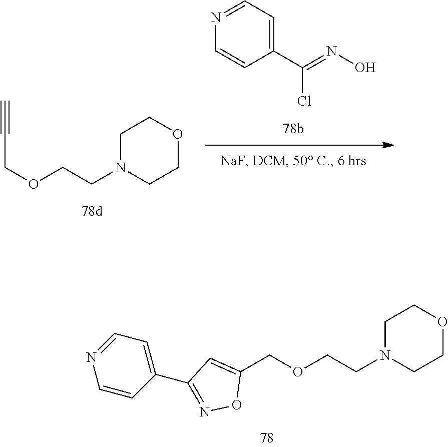 [SMILES: C#CCOCCN1CCOCC1 ; O/N=C(\Cl)c1ccncc1 ; c1cc(-c2cc(COCCN3CCOCC3)on2)ccn1]

Compound 78d (300 mg, 1.77 mmol, 1.00 eq) was dissolved in DCM (3.00 mL), and NaF (245 mg, 5.85 mmol, 245 μL, 3.30 eq) and compound 78b (333 mg, 2.13 mmol, 1.20 eq) were added. The mixture was stirred at 50° C. for 6 hours, and LCMS showed the complete reaction of compound 78d, with the target product's main peak (m/z=290.1, M+H⁺) being formed. The reaction mixture was concentrated under reduced pressure to obtain a crude product, which was purified (column: Phenomenex C18 250×50 mm×10 um; mobile phase: [water (ammonia hydroxide v/v)-ACN]; B %: 8%-38%, 8 mins) to yield a yellow solid compound 78 (110 mg, 374 μmol, yield 21.1%, purity 98.4%).

LCMS: m/z=290.1, M+H⁺

¹H NMR: (400 MHz, CDCl₃) δ 78.75 (d, J=5.6 Hz, 2H), 7.69 (d, J=6.0 Hz, 2H), 6.64 (s, 1H), 4.71 (s, 2H), 3.73-3.76 (m, 6H), 2.54-2.68 (n, 6H).

The preparation of compounds listed below is carried out with reference to Example 13

| Name | Structure | Data |
|---|---|---|
| Compound 83 | 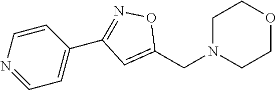 [SMILES: c1cc(-c2cc(CN3CCOCC3)on2)ccn1] | ¹H NMR (400 MHz, MeOD) δ 8.68 (d, J = 4.0 Hz, 1H), 7.89 (d, J = 6.0 Hz, 2H), 6.95 (s, 1H), 3.82 (s, 2H), 3.72 (t, J = 4.8 Hz, 4H), 2.58 (t, J = 4.4 Hz, 4H). |
| | | MS m/z(ESI): 246.1 [M + H]⁺. |
| | | HPLC purity: 98.2%. |
| Compound 84 | 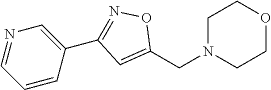 [SMILES: c1cncc(-c2cc(CN3CCOCC3)on2)c1] | ¹H NMR (400 MHz, CDCl₃) δ 9.01 (s, 1H), 8.70 (t, j = 1.6 Hz, 1H), 8.14-8.17 (m, 1H), 7.39-7.43 (m, 1H), 6.58 (s, 1H), 3.76 (t, j = 6.0 Hz, 6H), 2.58-2.62 (m, 4H). |
| | | MS m/z(ESI): 246.1 [M + H]⁺. |
| | | HPLC purity: 99.4%. |
| Compound 89 | 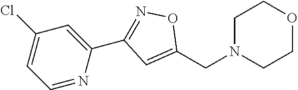 [SMILES: Clc1ccnc(-c2cc(CN3CCOCC3)on2)c1] | ¹H NMR (MeOD, 400 MHz) δ 8.61 (d, J = 5.2 Hz, 1H), 8.09 (d, J = 2.0 Hz, 1H), 7.55 (dd, J = 5.6 Hz, 1H), 6.94 (s, 1H), 3.81 (s, 2H), 3.71 (t, J = 4.4 Hz, 4H), |
| | | 2.58 (t, J = 4.4 Hz, 4H). |
| | | MS m/z(ESI): 280.1 [M + H]⁺. |
| | | HPLC purity: 100%. |
| Compound 92 | 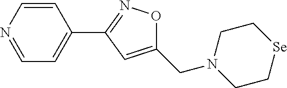 [SMILES: c1cc(-c2cc(CN3CC[Se]CC3)on2)ccn1] | ¹H NMR (400 MHz, CDCl₃) δ 8.75 (d, J = 3.2 Hz, 2H), 7.69 (t, J = 4.4 Hz, 2H), 6.61 (s, 1H), 3.84 (s, 2H), 3.01 (s, 4H), 2.80 (s, 4H). MS m/z(ESI): 310.0 [M + H]⁺. |
| | | HPLC purity: 97.6%. |
| Compound 95 | 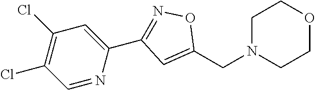 [SMILES: Clc1cnc(-c2cc(CN3CCOCC3)on2)cc1Cl] | ¹H NMR (400 MHz, CDCl₃) δ 8.68 (d, J = 2.4 Hz, 1H), 8.26 (d, J = 2.0 Hz, 1H), 6.57 (s, 1H), 3.74-3.77 (m, 6H), 2.58 (t, J = 4.4 Hz, 1H). |
| | | MS m/z(ESI): 314.1 [M + H]⁺. |
| | | HPLC purity: 98.5%. |

### Example 14: Preparation of Compound 98

### Preparation of Compound 98b

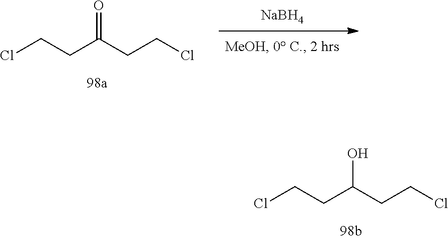 [SMILES: O=C(CCCl)CCCl ; "NaBH4" ; OC(CCCl)CCCl]

Compound 98a (500 mg, 3.23 mmol, 1.00 eq) was dissolved in MeOH (5.00 mL) and NaBH₄ (244 mg, 6.45 mmol, 2.00 eq) was added. The reaction was stirred at 0° C. for 2 hours, and TLC (PE:EA=2:1, R_{f}=0.50) monitored the complete reaction of compound 98a and the formation of a new spot. 5.00 mL H₂O was added to quench the reaction, and the mixture was extracted with ethyl acetate (5.00 mL×3), washed with saturated brine (5.00 mL×3), and dried over Na₂SO₄, filtered, and concentrated under reduced pressure to obtain a yellow oily compound 98b (450 mg, 2.87 mmol, yield 88.8%).

¹H NMR: (400 MHz, CDCl₃) δ 4.13-4.18 (m, 1H), 3.68-3.78 (m, 4H), 1.89-1.99 (m, 4H).

### Preparation of Compound 98c

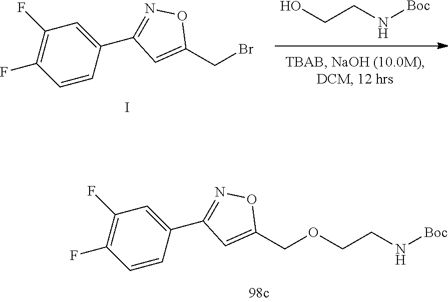

Intermediate I (2.00 g, 7.30 mmol, 1.00 eq) and tert-butyl (2-hydroxyethyl)carbamate (1.41 g, 8.76 mmol, 1.36 mL, 1.20 eq) were dissolved in DCM (20.0 mL), and TBAB (1.65 g, 5.11 mmol, 0.70 eq) and NaOH (10.0 M, 1.46 mL, 2.00 eq) were added. The reaction was stirred at 20° C. for 12 hours, and LCMS showed the complete reaction of Intermediate I and the formation of a new target molecule (m/z=377.3, M+Na⁺). The reaction mixture was extracted with 30.0 mL DCM, washed with saturated brine (30.0 mL), and the combined organic phase was dried over Na₂SO₄, filtered, and concentrated under reduced pressure to obtain a concentrate, which was purified by column chromatography (SiO₂, PE/EA=10/1 to 3/1, R_{f}=0.50) to yield a light-yellow oily compound 98c (1.00 g, 2.82 mmol, yield 38.7%).

LCMS: m/z=377.3, M+Na⁺

¹H NMR: (400 MHz, CDCl₃) δ 7.63-7.69 (m, 1H), 7.52-7.56 (m, 1H), 7.23-7.29 (m, 1H), 6.53 (s, 1H), 4.89 (s, 1H), 4.66 (s, 1H), 3.64 (t, J=4.8 Hz, 2H), 3.37-3.38 (m, 2H), 1.45 (s, 9H).

### Preparation of Compound 98d

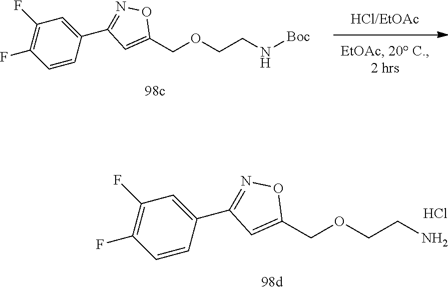 [SMILES: CC(C)(C)OC(=O)NCCOCc1cc(-c2ccc(F)c(F)c2)no1 ; NCCOCc1cc(-c2ccc(F)c(F)c2)no1 ; Cl]

Compound 98c (800 mg, 2.26 mmol, 1.00 eq) was dissolved in EtOAc (8.00 mL) and HCl/EtOAc (4.00 M, 5.64 mL, 10.0 eq) was added. The reaction was stirred at 20° C. for 2 hours, and LCMS showed the complete reaction of compound 98c, with a new target main peak (m/z=255.5, M+H⁺) generated. The reaction mixture was concentrated under reduced pressure to obtain a white solid compound 98d (750 mg, crude, HCl).

LCMS: m/z=255.5, M+H⁺

### Preparation of Compound 98

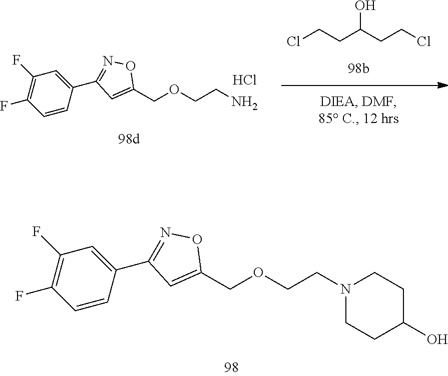 [SMILES: OC(CCCl)CCCl ; NCCOCc1cc(-c2ccc(F)c(F)c2)no1 ; Cl ; OC1CCN(CCOCc2cc(-c3ccc(F)c(F)c3)no2)CC1]

Compound 98d (200 mg, 688 μmol, 1.00 eq, HCl) was dissolved in DMF (2.00 mL), and DIEA (444 mg, 3.44 mmol, 599 μL, 5.00 eq) and compound 98b (130 mg, 825 μmol, 1.20 eq) were added. The mixture was stirred at 85° C. for 12 hours. LCMS showed the complete reaction of compound 98d, with a target main peak (m/z=339.3, M+H⁺) generated. The reaction mixture was filtered and concentrated under reduced pressure to obtain a crude product, which was then purified by preparative separation (column: Phenomenex Luna C18 150×30 mm×5 μm; mobile phase: [water (HCl)-ACN]; gradient: 15%-45% B over 10 min) to yield a yellow oily compound 98 (100 mg, 281 μmol, yield 40.8%, purity 95.0%).

LCMS: m/z=339.3, M+H⁺

¹H NMR: (400 MHz, MeOD) δ7.78-7.83 (m, 1H), 7.68-7.71 (m, 1H), 7.38-7.45 (m, 1H), 6.96 (s, 1H), 4.78-4.80 (m, 2H), 4.07-4.10 (m, 0.5H), 3.91-3.96 (m, 2H), 3.81-3.86 (m, 0.5H), 3.61-3.64 (m, 1H), 3.35-3.43 (m, 4H), 3.06-3.13 (m, 1H), 1.74-2.15 (m, 4H).

### Example 15: Preparation of Compound 99

### Preparation of Compound 99b

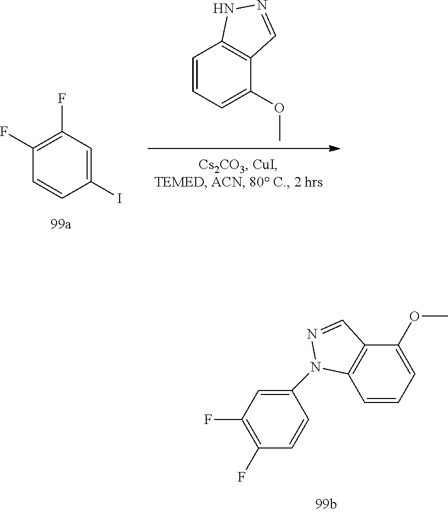 [SMILES: Fc1ccc(I)cc1F ; COc1cccc2[nH]ncc12 ; COc1cccc2c1cnn2-c1ccc(F)c(F)c1]

Compound 99a (1.15 g, 4.79 mmol, 1.00 eq), 4-methoxy-1H-indole (710 mg, 4.79 mmol, 1.00 eq), CuI (274 mg, 1.44 mmol, 0.30 eq), TEMED (111 mg, 958 μmol, 145 μL, 0.20 eq), and Cs₂CO₃ (3.12 g, 9.58 mmol, 2.00 eq) were dissolved in ACN (15.0 mL), and the mixture was purged with N₂ three times. Under nitrogen protection, the reaction was stirred at 85° C. for 2 hours. LCMS showed the complete reaction of compound 99a, with a new target main peak (m/z=261.1, M+H⁺) generated. The reaction mixture was added to 50.0 mL H₂O for phase separation, and the organic phase was separated. The aqueous phase was washed with 60.0 mL (30.0 mL×2) dichloromethane, dried over Na₂SO₄, filtered, and concentrated under reduced pressure to obtain a crude product, which was purified by column chromatography (SiO₂, PE:EA=10:1, R_{f}=0.46) to yield a yellow solid compound 99b (400 mg, 1.53 mmol, yield 31.9%, purity 99.3%).

LCMS: m/z=261.1, M+H⁺

¹H NMR: 400 MHz, CDCl₃ δ 8.27 (s, 1H), 7.50-7.51 (m, 1H), 7.47-7.50 (m, 1H), 7.27-7.40 (m, 3H), 6.59 (d, J=8.0 Hz, 1H), 4.01 (s, 3H).

### Preparation of Compound 99c

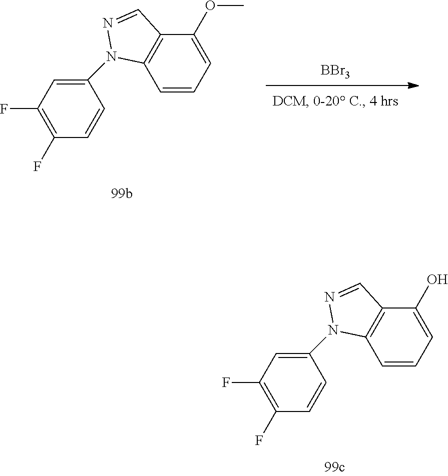 [SMILES: COc1cccc2c1cnn2-c1ccc(F)c(F)c1 ; "BBr3" ; Oc1cccc2c1cnn2-c1ccc(F)c(F)c1]

Compound 99b (100 mg, 384 μmol, 1.00 eq) was dissolved in DCM (1.00 mL), and BBr₃ (289 mg, 1.15 mmol, 111 μL, 3.00 eq) was added at 0° C. The reaction was stirred at 20° C. for 4 hours. LCMS showed the complete reaction of compound 99b, with a new target main peak (m/z=247.3, M+H⁺) generated. At 0° C., H₂O (3.00 mL) was added to the reaction mixture, which was then extracted, filtered, and concentrated under reduced pressure to obtain a white solid compound 99c (90.0 mg, 362 μmol, yield 94.2%, purity 99.1%).

LCMS: m/z=247.3, M+H⁺

### Preparation of Compound 99

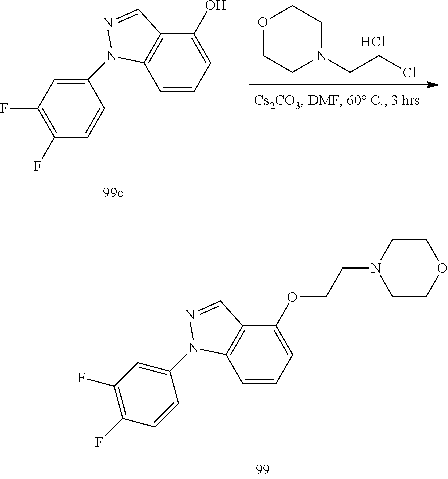 [SMILES: Oc1cccc2c1cnn2-c1ccc(F)c(F)c1 ; ClCCN1CCOCC1 ; Cl ; Fc1ccc(-n2ncc3c(OCCN4CCOCC4)cccc32)cc1F]

Compound 99c (100 mg, 406 μmol, 1.00 eq), N-(2-chloroethyl)morpholine hydrochloride (90.7 mg, 487 μmol, 1.20 eq), and Cs₂CO₃ (397 mg, 1.22 mmol, 3.00 eq) were dissolved in DMF (2.00 mL), and the atmosphere was replaced with nitrogen three times. The mixture was stirred under nitrogen protection at 60° C. for 3 hours. LCMS showed the complete reaction of compound 99c, with a new target main peak (m/z=360.4, M+H⁺) generated. DMF (2.00 mL) was removed under reduced pressure, and the concentrate was purified (column: Phenomenex Luna C18 150×25 mm×10 μm; mobile phase: [water (FA)-ACN]; B %: 11%-41%, 10 min) to obtain a white solid compound 99 (28.3 mg, 267 μmol, yield 65.6%, purity 95.8%).

LCMS: m/z=360.4, M+H⁺

¹H NMR: 400 MHz, CDCl₃ δ 8.24 (s, 1H), 7.36-7.59 (m, 1H), 7.34-7.34 (m, 1H), 7.27-7.34 (m, 3H), 6.58 (d, J=7.6 Hz, 1H), 4.36 (d, J=5.6 Hz, 2H), 3.774-3.78 (m, 4H), 2.96 (d, J=3.78 Hz, 2H), 2.69-2.72 (m, 4H).

### Example 16: Preparation of Compound 100

### Preparation of Compound 100b

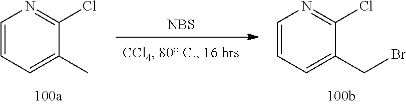

Compound 100a (5.00 g, 39.2 mmol, 4.27 mL, 1.00 eq) was dissolved in CCl₄ (50.0 mL), and BPO (94.9 mg, 392 μmol, 0.01 eq) and NBS (6.28 g, 35.3 mmol, 0.90 eq) were added. The mixture was stirred at 80° C. for 16 hours, and LCMS showed the formation of the target compound m/z (m/z=207.9, M+H⁺). The reaction mixture was concentrated under reduced pressure, and the concentrate was purified by column chromatography (SiO₂, PE:EA=10:1, R_{f}=0.56) to obtain a yellow oily compound 100b (3.30 g, 11.7 mmol, yield 29.9%, purity 73.2%).

LCMS: m/z=207.9, M+H⁺

### Preparation of Compound 100c

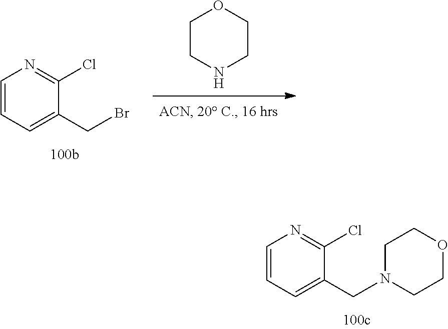 [SMILES: Clc1ncccc1CBr ; C1COCCN1 ; Clc1ncccc1CN1CCOCC1]

Compound 100b (3.30 g, 11.7 mmol, 1.00 eq) was dissolved in ACN (20.0 mL) and morpholine (2.04 g, 23.4 mmol, 2.06 mL, 2.00 eq) was added. The mixture was stirred at 20° C. for 2 hours, and LCMS showed the formation of the target compound (m/z=213.1, M+H⁺). The reaction mixture was diluted with 20 mL water and extracted with 20.0 mL ethyl acetate, washed with saturated saline (30.0 mL), dried over Na₂SO₄, filtered, and concentrated under reduced pressure to obtain a yellow oily crude product 100c (1.70 g, 7.40 mmol, yield 63.3%, purity 92.6%) without purification, directly proceeding to the next step.

LCMS: m/z=213.1, M+H⁺

### Preparation of Compound 100d

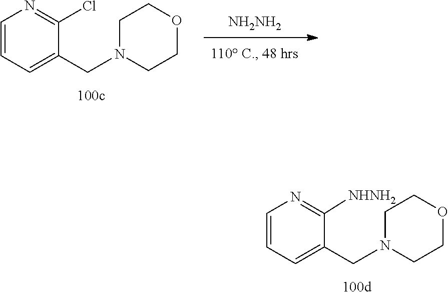 [SMILES: Clc1ncccc1CN1CCOCC1 ; "NH2NH2" ; NNc1ncccc1CN1CCOCC1]

At 20° C., compound 100c (500 mg, 2.18 mmol, 1.00 eq) was dissolved in hydrazine hydrate (1.22 g, 24.0 mmol, 1.19 mL, 98.0% purity, 11.0 eq). The mixture was stirred at 110° C. for 24 hours, and LCMS showed the formation of the target compound (m/z=209.1, M+H⁺). The reaction mixture was concentrated under reduced pressure, and the crude product was used in the next step without purification, yielding a yellow oily compound 100d (500 mg, crude product).

LCMS: m/z=209.1, M+H⁺

### Preparation of Compound 100e

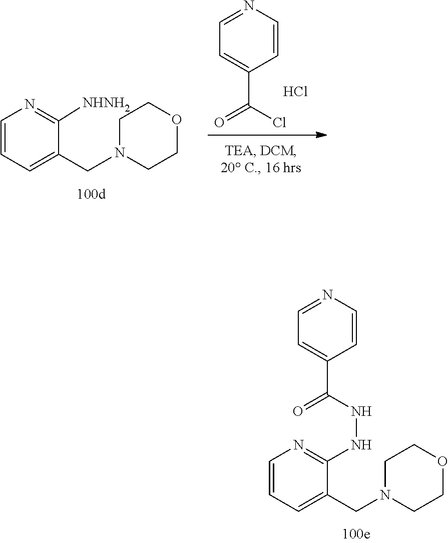

Compound 100d (200 mg, 960 μmol, 1.00 eq) was dissolved in DCM (2.00 mL), and TEA (97.2 mg, 960 μmol, 134 μL, 1.00 eq) and chloroisocyanuric acid chloride (154 mg, 864 μmol, 0.90 eq, HCl) were added. The mixture was stirred at 20° C. for 2 hours, and LCMS showed the formation of the target compound (m/z=314.1, M+H⁺). The reaction mixture was concentrated under reduced pressure, and the crude product was used in the next step without purification, yielding a yellow oily compound 100e (300 mg, crude product).

LCMS: m/z=314.1, M+H⁺

### Preparation of Compound 100

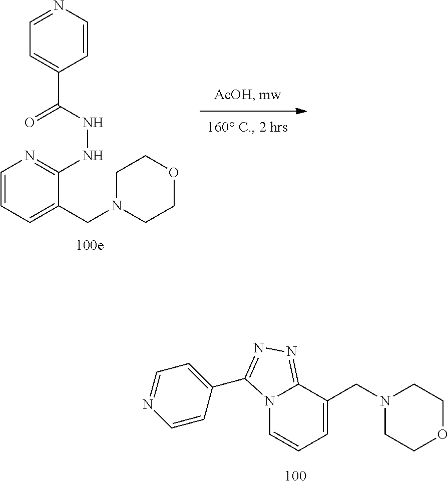 [SMILES: O=C(NNc1ncccc1CN1CCOCC1)c1ccncc1 ; c1cc(CN2CCOCC2)c2nnc(-c3ccncc3)n2c1]

Compound 100e (300 mg, 777 μmol, 1.00 eq) was placed in a microwave reaction tube, and AcOH (3.00 mL) was added. The tube was sealed and heated in a microwave to 160° C. for 3 hours. LCMS showed the formation of the target compound (m/z=296.2, M+H⁺). The reaction mixture was concentrated under reduced pressure, and the crude product was purified using a basic condition column (Phenomenex C18 250×50 mm×10 μm; mobile phase: [water (NH₃·H₂O)-ACN]; B %: 1%-25%, 10 min) to yield a white solid compound 100 (150 mg, 508 μmol, yield 50.0%, purity 100%).

LCMS: m/z=296.2, M+H⁺

¹H NMR: (400 MHz, CDCl₃) δ 8.87 (d, J=6.0 Hz, 2H), 8.29 (d, J=6.8 Hz, 1H), 7.81-7.82 (m, 2H), 7.47 (s 1H), 7.00 (t, J=6.8 Hz, 1H), 4.11 (s, 2H), 3.80 (s, 4H), 2.68 (s, 4H).

### Example 17: Preparation of Compound 101

### Preparation of Compound 101b

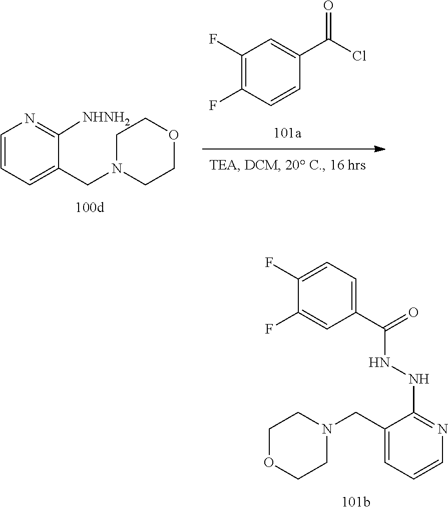 [SMILES: O=C(Cl)c1ccc(F)c(F)c1 ; NNc1ncccc1CN1CCOCC1 ; O=C(NNc1ncccc1CN1CCOCC1)c1ccc(F)c(F)c1]

Compound 100d (250 mg, 1.20 mmol, 1.00 eq) was dissolved in DCM (2.00 mL) and compound 101a (191 mg, 1.08 mmol, 135 μL, 0.90 eq) was added. The reaction mixture was stirred at 20° C. for 16 hours. LCMS showed the formation of the target compound (m/z=349.1, M+H⁺). The reaction mixture was concentrated under reduced pressure to yield a yellow solid 101b (250 mg, crude product).

LCMS: MS=349.1, M+H⁺

### Preparation of Compound 101

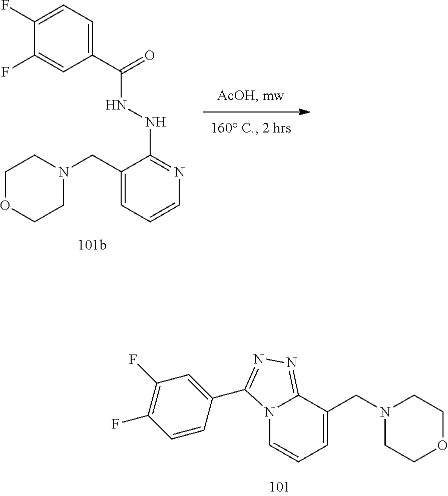 [SMILES: O=C(NNc1ncccc1CN1CCOCC1)c1ccc(F)c(F)c1 ; Fc1ccc(-c2nnc3c(CN4CCOCC4)cccn23)cc1F]

Compound 101b (157 mg, 452 μmol, 1.00 eq) was placed in a microwave reaction tube and dissolved in AcOH (2.50 mL). The tube was sealed and heated in a microwave to 160° C. for 2 hours. LCMS showed the formation of the target compound (m/z=331.1, M+H⁺). The reaction mixture was concentrated under reduced pressure to obtain a concentrate, which was then treated with NH₃·H₂O (3.00 mL) and filtered. The filter cake was dried under reduced pressure to yield a yellow solid compound 101 (140 mg, 415 μmol, yield 91.8%, purity 97.9%).

LCMS: m/z: 331.1, M+H⁺

¹HNMR (400 MHz, CDCl₃) δ 8.14-8.16 (d, J=6.8 Hz, 1H), 7.61-7.70 (m, 1H), 7.58-7.59 (m, 1H), 7.44-7.58 (m, 2H), 6.96 (t, J=6.8 Hz, 1H), 4.08 (s, 2H), 3.79 (s, 4H), 2.63 (s, 4H).

### Example 18: Preparation of Compound 103

### Preparation of Compound 103b

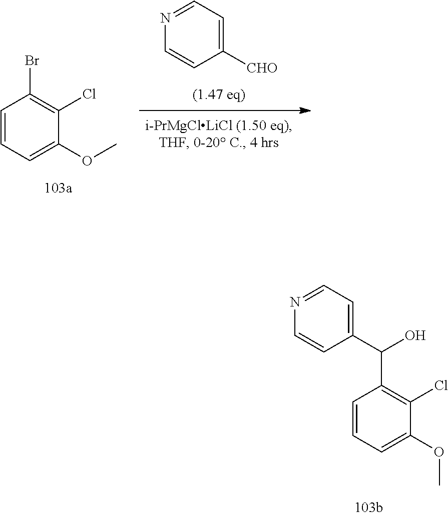

Compound 103a (1.00 g, 4.52 mmol, 1.00 eq) was dissolved in THE (10.0 mL). At 0° C., i-PrMgCl·LiCl (1.3 M, 5.21 mL, 1.50 eq) was added and the reaction was stirred for 1 hour. Then, 4-pyridinecarboxaldehyde (713 mg, 6.66 mmol, 627 μL, 1.47 eq) was added while maintaining the temperature at 0° C., and the reaction continued for 3 hours. LCMS showed complete consumption of 4-pyridinecarboxaldehyde and the formation of a new target peak (MS=250.3, M+H⁺). The reaction mixture was quenched with NH₄Cl (10.0 mL), extracted with EtOAc (10.0 mL×3), washed with saturated brine (10.0 mL×3), dried over Na₂SO₄, filtered, and concentrated under reduced pressure to obtain a concentrate. Preparative thin-layer chromatography (PE/EA=2/1, R_{f}=0.26) yielded a brown oily compound 103b (860 mg, 3.07 mmol, yield 68.0%, purity 89.1%).

LCMS: MS=250.3, M+H⁺

### Preparation of Compound 103c

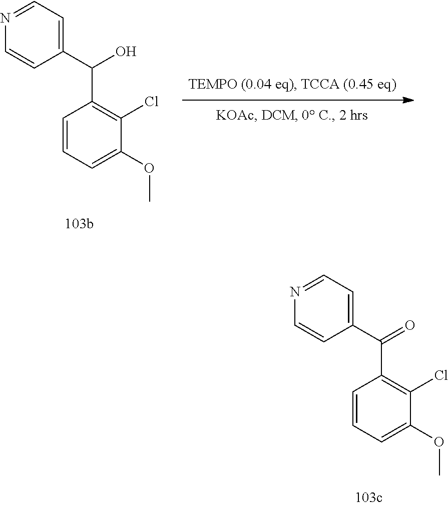 [SMILES: COc1cccc(C(O)c2ccncc2)c1Cl ; COc1cccc(C(=O)c2ccncc2)c1Cl]

Compound 103b (860 mg, 3.07 mmol, 1.00 eq) was dissolved in DCM (9.00 mL). At 0° C., TEMPO (19.3 mg, 123 μmol, 0.04 eq) and KOAc (376 mg, 3.84 mmol, 1.25 eq) were added, followed by dropwise addition of TCCA (321 mg, 1.38 mmol, 0.45 eq). The reaction was maintained at 0° C. and stirred for 2 hours. LCMS showed complete reaction of compound 103b and the formation of a new target compound (MS=248.4, M+H⁺). The reaction mixture was filtered through diatomaceous earth, and the filter cake was washed with DCM. Preparative thin-layer chromatography (PE/EA=2/1, R_{f}=0.48) yielded a brown oily compound 103c (570 mg, 1.99 mmol, yield 64.9%, purity 86.5%).

LCMS: MS=248.3, M+H⁺

### Preparation of Compound 103d

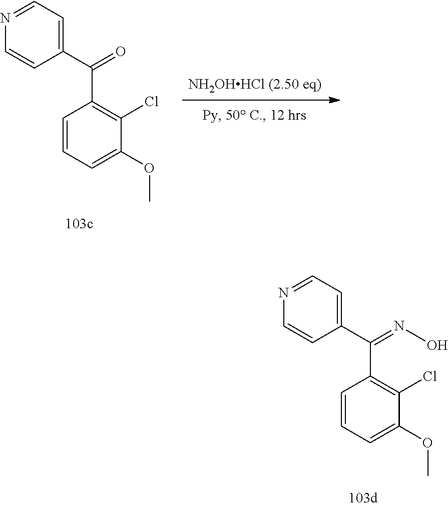 [SMILES: COc1cccc(C(=O)c2ccncc2)c1Cl ; COc1cccc(/C(=N\O)c2ccncc2)c1Cl]

Compound 103c (570 mg, 1.99 mmol, 1.00 eq) was dissolved in pyridine (5.00 mL), and NH₂OH·HCl (346 mg, 4.98 mmol, 2.50 eq) was added. The mixture was stirred at 50° C. for 12 hours. LCMS indicated the complete reaction of compound 103c and the formation of a new target compound (MS=263.4, M+H⁺). Pyridine was removed under reduced pressure, and the residue was diluted with 5.00 mL H₂O, then extracted with EtOAc (5.00 mL×3). The combined organic layers were washed with saturated brine (5.00 mL×2), dried over Na₂SO₄, filtered, and concentrated under reduced pressure to yield a yellow solid compound 103d (500 mg, 1.85 mmol, yield 93.0%, purity 97.3%).

LCMS: MS=263.4, M+H⁺

### Preparation of Compound 103e

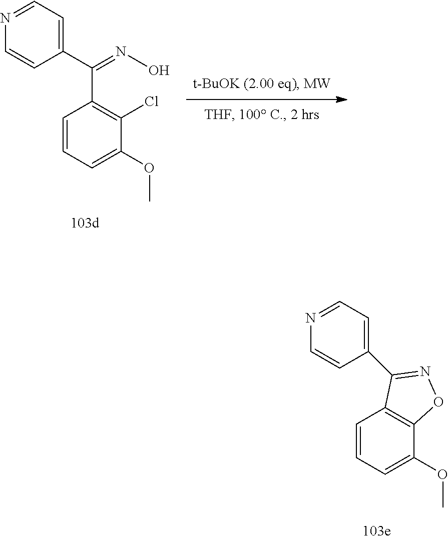 [SMILES: COc1cccc(/C(=N\O)c2ccncc2)c1Cl ; COc1cccc2c(-c3ccncc3)noc12]

Compound 103d (200 mg, 741 μmol, 1.00 eq) and t-BuOK (1 M, 1.48 mL, 2.00 eq) were added to a microwave reaction tube, dissolved in THE (2.00 mL), and the tube was sealed and heated in a microwave to 100° C. for 2 hours. LCMS showed that compound 103d was partially reacted, and a new target compound (MS=227.3, M+H⁺) was formed. The reaction mixture was concentrated under reduced pressure, and the residue was purified by thin-layer chromatography (PE:EA=1/1, R_{f}=0.18) to obtain a light-yellow solid compound 103e (90.0 mg, 306 μmol, yield 41.3%, purity 77.0%).

LCMS: MS=227.3, M+H⁺

### Preparation of Compound 103f

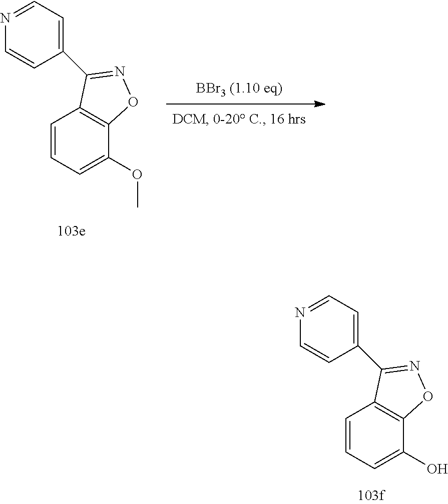 [SMILES: COc1cccc2c(-c3ccncc3)noc12 ; Oc1cccc2c(-c3ccncc3)noc12]

Compound 103e (90.0 mg, 398 μmol, 1.00 eq) was dissolved in DCM (1.00 mL), cooled to 0° C., and BBr₃ (109 mg, 437 μmol, 42.2 μL, 1.10 eq) was added. The mixture was stirred at 20° C. for 10 hours. LCMS showed that compound 103e was fully reacted, producing a new target product peak (MS=213.3, M+H⁺). The reaction was quenched with 1 mL water, stirred for 0.5 hours at 20° C., and the mixture was concentrated under reduced pressure to obtain a yellow solid compound 103f (130 mg, crude).

LCMS: MS=213.3, M+H⁺

### Preparation of Compound 103

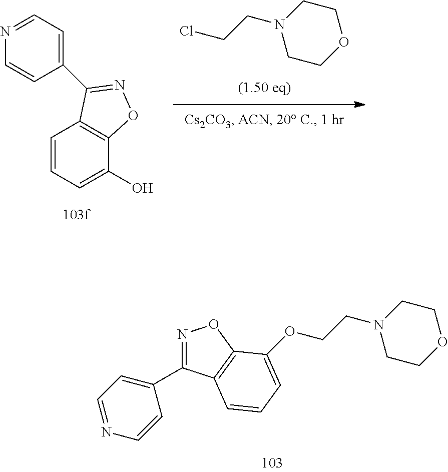 [SMILES: Oc1cccc2c(-c3ccncc3)noc12 ; ClCCN1CCOCC1 ; c1cc(OCCN2CCOCC2)c2onc(-c3ccncc3)c2c1]

Compound 103f (130 mg, 613 μmol, 1.00 eq) was dissolved in ACN (1.30 mL) and compound 103g (137 mg, 919 μmol, 1.50 eq) and Cs₂CO₃ (399 mg, 1.23 mmol, 2.00 eq) were added. The mixture was stirred at 20° C. for 1 hour. LCMS showed that compound 103f reacted completely, producing a new target peak (MS=326.3, M+H⁺). The reaction mixture was filtered, concentrated under reduced pressure, and purified (column: Waters xbridge 150×25 mm×10 μm; mobile phase: [water (NH₃·H₂O)-ACN]; B %: 25%-45%, 10 min) to obtain a gray-white solid compound 103 (60.0 mg, 182 μmol, yield 29.7%, purity 98.6%).

LCMS: MS=326.3, M+H⁺

¹H NMR: (400 MHz, CDCl₃) δ 8.85 (t, J=4.8 Hz, 2H), 7.88 (t, J=4.4 Hz, 2H), 7.51 (d, J=8.0 Hz, 1H), 7.35 (t, J=8 Hz, 1H), 7.10 (d, J=7.6 Hz, 1H), 4.46 (s, 2H), 3.77 (s, 4H), 2.97 (s, 2H), 2.69 (s, 4H).

### Example 19: Preparation of Compound 106

### Preparation of Compound 106b

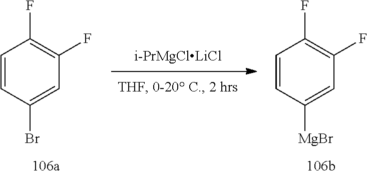

Compound 106a (1.00 g, 5.18 mmol, 1.00 eq) was dissolved in THE (10.0 mL), and i-PrMgCl—LiCl (1.50 M, 5.18 mL, 1.50 eq) was added at 0° C. The reaction mixture was stirred at 20° C. for 2 hours. HPLC showed that the starting material reacted completely. The reaction mixture was used for the next step without purification.

### Preparation of Compound 106c

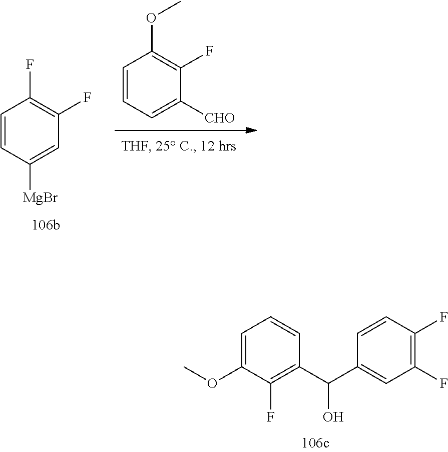

Compound 106b (1.13 g, 5.20 mmol, 1.17 mL, 1.00 eq) was dissolved in THE (10.0 mL), and 2-fluoro-3-methoxybenzaldehyde (961 mg, 6.24 mmol, 1.20 eq) was added. The reaction mixture was stirred at 25° C. for 12 hours. LCMS showed that compound 106b reacted completely, generating a new target peak. The reaction mixture was quenched with NH₄Cl (10.0 mL), extracted with EtOAc (10.0 mL×3), washed with saturated brine (10.0 mL×3), dried over Na₂SO₄, filtered, and concentrated under reduced pressure to obtain a yellow oily compound 106c (900 mg, 3.36 mmol, yield 64.5%).

### Preparation of Compound 106d

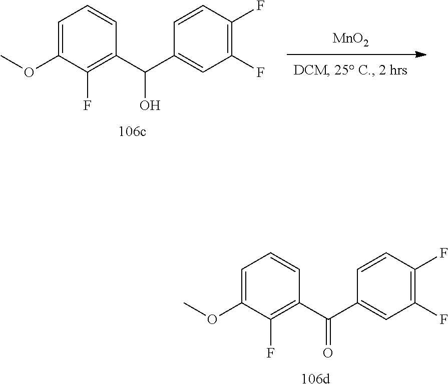 [SMILES: COc1cccc(C(O)c2ccc(F)c(F)c2)c1F ; "MnO2" ; COc1cccc(C(=O)c2ccc(F)c(F)c2)c1F]

Compound 106c (500 mg, 1.86 mmol, 1.00 eq) was dissolved in DCM (8.00 mL) and MnO₂ (1.62 g, 18.6 mmol, 10.0 eq) was added. The reaction mixture was stirred at 25° C. for 2 hours, showing complete reaction of compound 106c, and generating a new target peak LCMS (m/z=267.4, M+H⁺). The reaction mixture was concentrated under reduced pressure to obtain a yellow oily compound 106d (500 mg, crude product).

LCMS: m/z=267.4, M+H⁺

### Preparation of Compound 106e

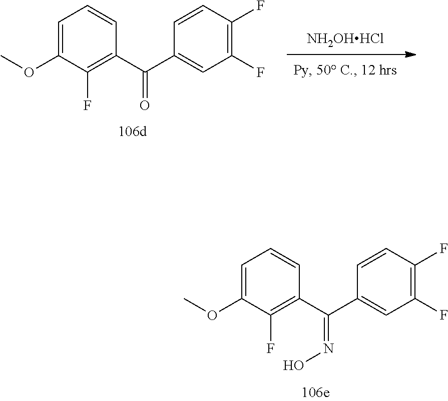 [SMILES: COc1cccc(C(=O)c2ccc(F)c(F)c2)c1F ; "NH2OH·HCl" ; COc1cccc(/C(=N\O)c2ccc(F)c(F)c2)c1F]

Compound 106d (500 mg, 1.88 mmol, 1.00 eq) was dissolved in pyridine (5.00 mL) and hydroxylamine hydrochloride (NH₂OH·HCl, 326 mg, 4.70 mmol, 2.50 eq) was added. The reaction mixture was stirred at 50° C. for 12 hours. LC-MS showed complete reaction of compound 106d and the generation of a new target peak (m/z=282.3, M+H⁺). The reaction mixture was concentrated under reduced pressure to obtain a yellow solid compound 106e (326 mg, 1.15 mmol, yield 61.2%, purity 99.2%).

LCMS: m/z=282.3, M+H⁺

¹H NMR: (400 MHz, CDCl₃) δ 7.47-7.53 (m, 1H), 7.38-7.43 (m, 1H), 7.18-7.22 (m, 2H), 6.95-6.99 (m, 1H), 6.77-6.81 (m, 1H), 3.95 (s, 3H).

### Preparation of Compound 106f

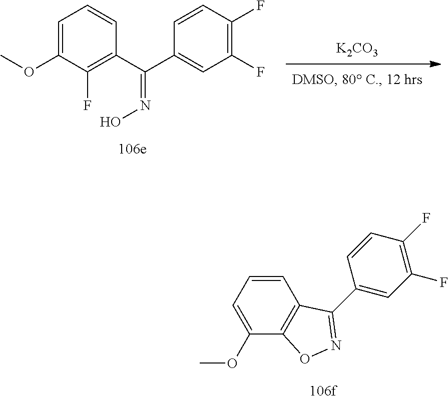 [SMILES: COc1cccc(/C(=N\O)c2ccc(F)c(F)c2)c1F ; "K2CO3" ; COc1cccc2c(-c3ccc(F)c(F)c3)noc12]

Compound 106e (230 mg, 817 μmol, 1.00 eq) was dissolved in DMSO (2.50 mL) and K₂C03 (169 mg, 1.23 mmol, 1.50 eq) was added. The reaction was stirred at 80° C. for 12 hours. LC-MS showed complete reaction of compound 106e and the generation of a new target peak (m/z=262.3, M+H⁺). At 25° C., the reaction was quenched with 5.00 mL water and extracted with ethyl acetate (EtOAc, 5.00 mL×3). The organic phase was washed with saturated brine (5.00 mL×3), dried over sodium sulfate (Na₂SO₄), filtered, and concentrated under reduced pressure to obtain a brown solid compound 106f (170 mg, 633 μmol, yield 77.5%, purity 97.4%).

LCMS: m/z=262.3, M+H⁺

¹H NMR: (400 MHz, CDCl₃) δ 7.72-7.85 (m, 2H), 7.31-7.45 (m, 3H), 7.05 (d, J=7.6, 1H), 4.09 (s, 3H).

### Preparation of Compound 106g

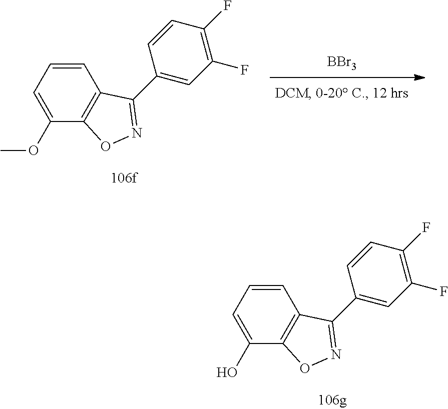 [SMILES: COc1cccc2c(-c3ccc(F)c(F)c3)noc12 ; "BBr3" ; Oc1cccc2c(-c3ccc(F)c(F)c3)noc12]

Compound 106f (170 mg, 650 μmol, 1.00 eq) was dissolved in dichloromethane (DCM, 2.00 mL), and at 0° C., boron tribromide (BBr₃, 489 mg, 1.95 mmol, 188 μL, 3.00 eq) was added. The reaction was stirred at 20° C. for 12 hours. LC-MS showed complete reaction of compound 106f and the generation of a new target peak (m/z=248.3, M+H⁺). At 0° C., the reaction was quenched with H₂O (3.00 mL), then extracted, filtered, and concentrated to obtain a yellow solid compound 106g (172 mg, crude).

LCMS: m/z=248.3, M+H⁺

¹H NMR: EC6663-444-P1A (400 MHz, CDCl₃) δ 7.72-7.85 (m, 2H), 7.29-7.45 (m, 3H), 7.12-7.14 (m, 1H).

### Preparation of Compound 106

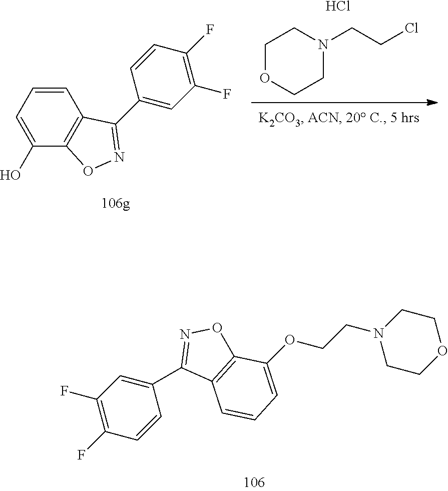

Compound 106g (170 mg, 687 μmol, 1.00 eq) was dissolved in acetonitrile (ACN, 2.00 mL), and K₂C03 (142 mg, 1.03 mmol, 1.50 eq) and 4-(2-chloroethyl)morpholine (123 mg, 825 μmol, 1.20 eq) were added. The reaction mixture was stirred at 20° C. for 5 hours. LC-MS showed complete reaction of compound 106g and the generation of a new target peak (m/z=361.4, M+H⁺). At 25° C., the reaction was quenched with 10 mL water, filtered, and concentrated under reduced pressure to obtain a brown solid compound 106 (165 mg, 452 μmol, yield 65.7%, purity 98.2%).

LCMS: m/z=361.4, M+H⁺

¹H NMR: (400 MHz, DMSO-d₆) δ 8.75 (d, J=5.6 Hz, 2H), 7.69 (d, J=6.0 Hz, 2H), 6.64 (s, 1H), 4.71 (s, 2H), 3.73-3.76 (m, 6H), 2.54-2.68 (m, 6H).

### Example 20: Preparation of Compound 107

### Preparation of Compound 107b

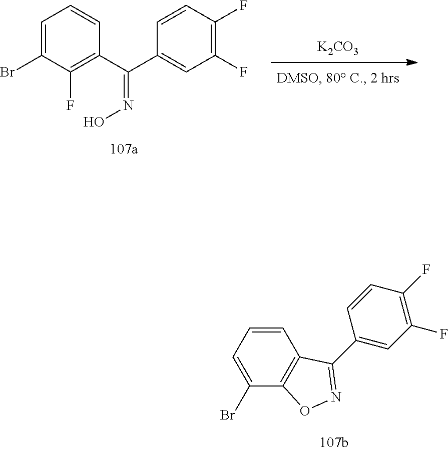 [SMILES: O/N=C(/c1ccc(F)c(F)c1)c1cccc(Br)c1F ; "K2CO3" ; Fc1ccc(-c2noc3c(Br)cccc23)cc1F]

Compound 107a (500 mg, 1.51 mmol, 1.00 eq) was dissolved in DMSO (5.00 mL) and added dropwise to K₂CO₃ (314 mg, 2.27 mmol, 1.50 eq). The mixture was heated to 80° C. and stirred for 2 hours. TLC monitoring revealed the formation of a new spot, which prompted preparative purification (PE:EA=2:1, R_{f} (P1)=0.28, R_{f} (R¹)=0.73). The reaction mixture was diluted with 10.0 mL water and extracted with ethyl acetate (10.0 mL×3). The organic phase was washed with saturated brine (10.0 mL), dried over sodium sulfate (Na₂SO₄), filtered, and concentrated under reduced pressure to yield a yellow solid crude product 107b (500 mg, crude), which was used directly in the next reaction step without further purification.

### Preparation of Compound 107c

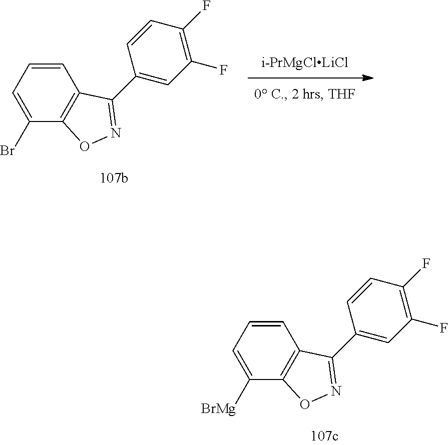

Compound 107b (200 mg, 645 μmol, 1.00 eq) was dissolved in THE (2.00 mL) and cooled to 0° C. i-PrMgCl—LiCl (1.30 M, 744 μL, 1.50 eq) was then added, and the mixture was maintained at 0° C. and stirred for 2 hours. HPLC analysis showed complete reaction of the starting material. The crude product, a colorless liquid 107c (215.0 mg, crude), was used directly in the next step without purification.

### Preparation of Compound 107d

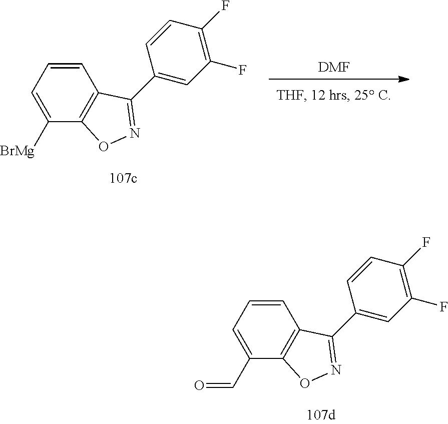

Compound 107c (215 mg, 642 μmol, 1.00 eq) was dissolved in THE (2.00 mL) and DMF (93.9 mg, 1.29 mmol, 98.9 μL, 2.00 eq) was added. The mixture was stirred at 20° C. for 12 hours. LC-MS analysis showed complete reaction of compound 107c and the generation of a new target peak (m/z=260.3, M+H⁺). The reaction mixture was concentrated under reduced pressure to obtain a brown solid crude product 107d (200 mg, crude), which was used directly in the next step without purification.

LCMS: m/z=260.3, M+H⁺

### Preparation of Compound 107

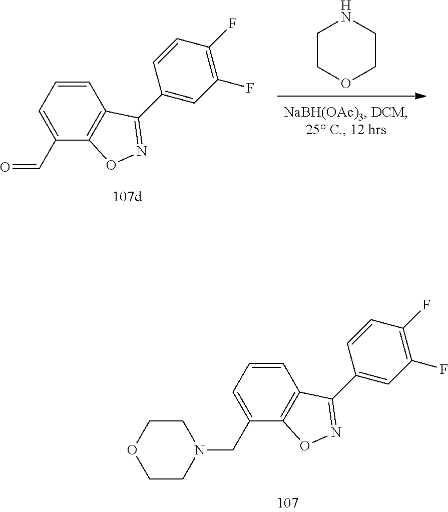 [SMILES: O=Cc1cccc2c(-c3ccc(F)c(F)c3)noc12 ; C1COCCN1 ; Fc1ccc(-c2noc3c(CN4CCOCC4)cccc23)cc1F]

Compound 107d (100 mg, 385 μmol, 1.00 eq) was dissolved in methanol (MeOH, 0.20 mL), NaBH₃CN (72.7 mg, 1.16 mmol, 3.00 eq) and morpholine (40.3 mg, 462 μmol, 40.7 μL, 1.20 eq) were added. The reaction mixture was stirred at 20° C. for 16 hours. LC-MS analysis showed complete reaction of compound 107d and the generation of a new target peak. After filtering and concentrating under reduced pressure, the concentrate was purified using a column (Phenomenex Luna C18, 150×25 mm×10 μm; mobile phase: [water (FA)-ACN]; gradient: 8%-38% B over 10 min) to obtain a white solid compound 107 (50.0 mg, 145 μmol, yield 18.9%, purity 96.4%).

LCMS: m/z=331.4, M+H⁺

¹H NMR: (400 MHz, DMSO-d₆) δ 8.05-8.13 (m, 2H), 7.81-7.85 (m, 1H), 7.67-7.74 (m, 2H), 7.46-7.50 (m, 1H), 3.81 (s, 2H), 3.52-3.65 (m, 4H), 2.35-2.41 (m, 4H).

### Example 21: Preparation of Compound 102

### Preparation of Compound 102b

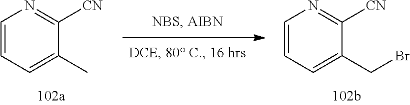

Compound 102a (5.00 g, 42.3 mmol, 1.00 eq), NBS (11.3 g, 63.5 mmol, 1.50 eq), and AIBN (6.95 g, 42.3 mmol, 1.00 eq) were dissolved in DCE (50.0 mL). The mixture was purged with nitrogen three times and stirred under light exclusion at 80° C. for 16 hours. LC-MS analysis showed the formation of the target compound (m/z=196.0, M+H⁺). The reaction mixture was filtered and concentrated under reduced pressure to obtain a crude yellow oily substance 102b (5.20 g, 26.4 mmol, yield 62.4%).

LCMS: m/z=197.2, M+H⁺

### Preparation of Compound 102c

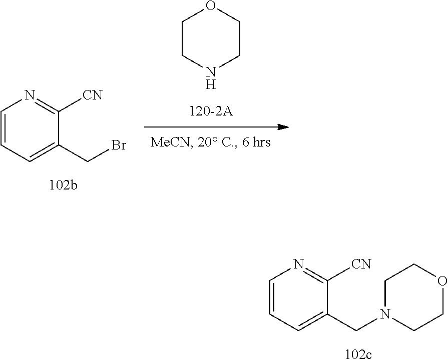 [SMILES: C1COCCN1 ; N#Cc1ncccc1CBr ; N#Cc1ncccc1CN1CCOCC1]

Compound 102b (5.00 g, 25.3 mmol, 1.00 eq) and morpholine (4.42 g, 50.7 mmol, 4.47 mL, 2.00 eq) were dissolved in CH₃CN (50.0 mL). The mixture was purged with nitrogen three times and stirred at 20° C. for 6 hours. LC-MS analysis indicated the formation of the target compound (m/z=204.1, M+H⁺). The reaction mixture was quenched with water (60.0 mL) and extracted three times with ethyl acetate (60.0 mL). The combined organic layers were washed three times with brine (60.0 mL), dried over sodium sulfate (Na₂SO₄), filtered, and concentrated under reduced pressure to obtain a yellow oily substance 102c (4.90 g, 24.1 mmol).

LCMS: m/z=204.1, M+H⁺

### Preparation of Compound 102d

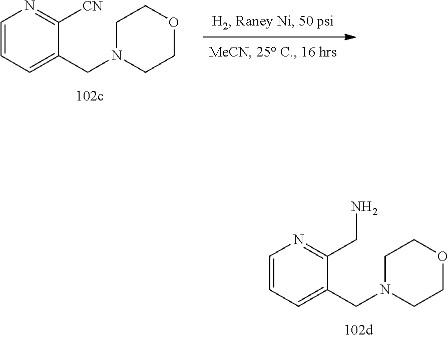 [SMILES: N#Cc1ncccc1CN1CCOCC1 ; NCc1ncccc1CN1CCOCC1]

Compound 102c (4.90 g, 24.1 mmol, 1.00 eq) was dissolved in methanol (MeOH, 50.0 mL) under an argon atmosphere, and Raney-Ni (980 mg, 11.4 mmol, 0.10 eq) was added. The mixture was purged three times with hydrogen and stirred under hydrogen gas (50 Psi) at 25° C. for 16 hours. LC-MS analysis indicated the formation of the target compound (m/z=208.2, M+H⁺). The reaction mixture was filtered and washed three times with methanol (50.0 mL), then concentrated to obtain a brown oily substance 102d (4.50 g, 21.7 mmol).

LCMS: m/z=208.2, M+H⁺

### Preparation of Compound 102e

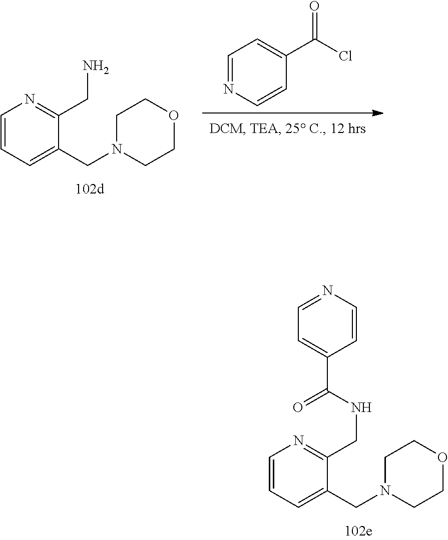 [SMILES: NCc1ncccc1CN1CCOCC1 ; O=C(Cl)c1ccncc1 ; O=C(NCc1ncccc1CN1CCOCC1)c1ccncc1]

Compound 102d (600 mg, 2.89 mmol, 1.00 eq) was dissolved in DCM (6.00 mL), and TEA (585 mg, 5.79 mmol, 805 μL, 2.00 eq) and isonicotinyl chloride (368 mg, 2.61 mmol, 0.90 eq) were added. The mixture was stirred at 25° C. for 12 hours. LC-MS analysis indicated the formation of the target compound (m/z=313.2, M+H⁺). The reaction mixture was filtered and concentrated under reduced pressure to obtain a brown oily crude product 102e (1.00 g).

LCMS: m/z=313.2, M+H⁺

### Preparation of Compound 102

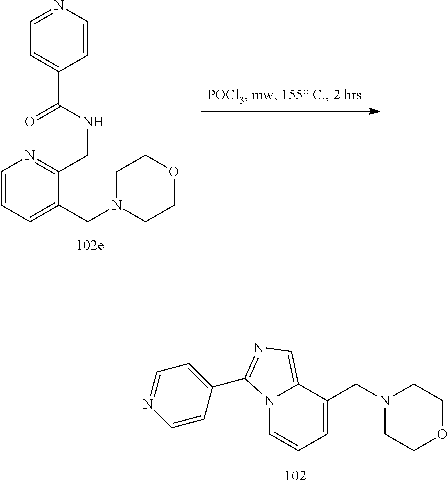 [SMILES: O=C(NCc1ncccc1CN1CCOCC1)c1ccncc1 ; c1cc(CN2CCOCC2)c2cnc(-c3ccncc3)n2c1]

Compound 102e (500 mg, 1.60 mmol, 1.00 eq) and phosphorus oxychloride (POCl₃, 3.00 mL) were placed in a microwave-sealed tube and heated under microwave conditions at 155° C. for 2 hours. LC-MS analysis indicated the formation of the target compound (m/z=295.2, M+H⁺). After cooling the reaction mixture to room temperature, water (4.00 mL) was added to quench the reaction. The mixture was filtered and concentrated under reduced pressure to obtain a crude product, which was purified by reverse-phase HPLC (column: Phenomenex C18 250×50 mm×10 μm; mobile phase: [water (NH₄HCO₃)-ACN]; B %: 10%-40%, 8 mins). A white solid compound 102 (205 mg, 685 μmol) was obtained, yielding 42.8% with a purity of 98.4%.

LCMS: m/z=295.2, M+H⁺

¹H NMR: (400 MHz, CDCl₃) δ 8.77 (d, J=5.6 Hz, 1H), 8.32 (d, J=6.8 Hz, 1H), 7.83-7.86 (m, 1H), 7.77 (d, J=6.0 Hz, 1H), 6.86 (d, J=6.4 Hz, 1H), 6.69 (t, J=7.2 Hz, 1H), 3.71-3.76 (m, 6H), 2.55 (s, 4H).

### Example 22: Preparation of Compound 104

### Preparation of Compound 104b

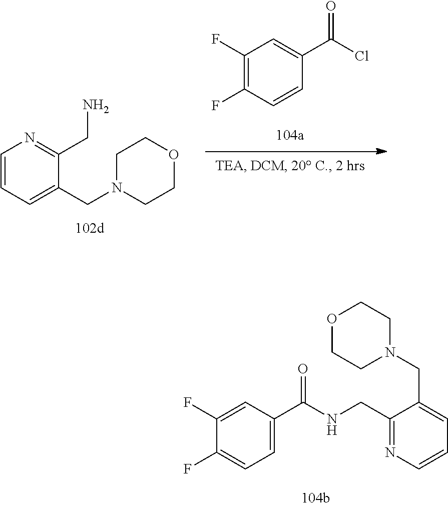 [SMILES: NCc1ncccc1CN1CCOCC1 ; O=C(Cl)c1ccc(F)c(F)c1 ; O=C(NCc1ncccc1CN1CCOCC1)c1ccc(F)c(F)c1]

Compound 102d (500 mg, 2.41 mmol, 1.00 eq) was dissolved in DCM (5.00 mL), and compound 104a (383 mg, 2.17 mmol, 271 μL, 0.90 eq) was added. The reaction mixture was stirred at 20° C. for 2 hours. LC-MS analysis indicated the formation of the target compound (m/z=348.1, M+H⁺). The reaction mixture was filtered and concentrated under reduced pressure to obtain a crude yellow solid 104b (800 mg, 2.16 mmol), yielding 89.6% with a purity of 93.9%.

LCMS: m/z=348.1, M+H⁺

### Preparation of Compound 104

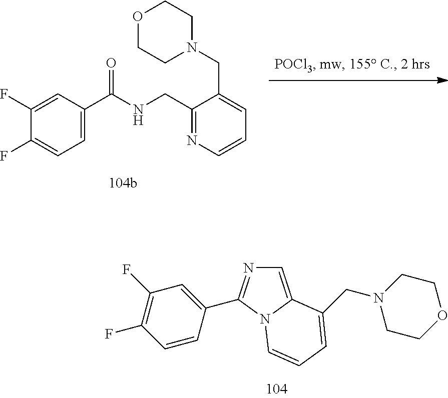 [SMILES: O=C(NCc1ncccc1CN1CCOCC1)c1ccc(F)c(F)c1 ; Fc1ccc(-c2ncc3c(CN4CCOCC4)cccn23)cc1F]

Compound 104b (500 mg, 1.44 mmol, 1.00 eq) and POCl₃ (3.00 mL) were added to a microwave-sealed tube and heated under microwave condition at 155° C. for 2 hours. LC-MS analysis indicated the formation of the target compound (m/z=330.1, M+H⁺). After cooling the reaction mixture to room temperature, water (5.00 mL) was added to quench the reaction. The mixture was filtered and concentrated under reduced pressure to obtain a crude product, which was purified by reverse-phase HPLC (column: Phenomenex C18 250×50 mm×10 μm; mobile phase: [water (NH₄HCO₃)-ACN]; B %: 29%-59%, 8 mins). This resulted in a yellow solid compound 104 (265 mg, 803 μmol), yielding 55.8% with a purity of 99.9%.

LCMS: m/z=330.1, M+H⁺

¹H NMR: (400 MHz, CDCl₃) δ 8.14 (d, J=7.2 Hz, 1H), 7.77 (s, 1H), 7.54-7.64 (m, 1H), 7.53-7.54 (m, 1H), 7.31-7.34 (m, 1H), 6.78 (d, J=6.4 Hz, 1H), 6.61 (t, J=6.4 Hz, 1H), 3.69-3.75 (m, 6H), 2.54 (s, 4H).

### Example 23: Preparation of Compound 105

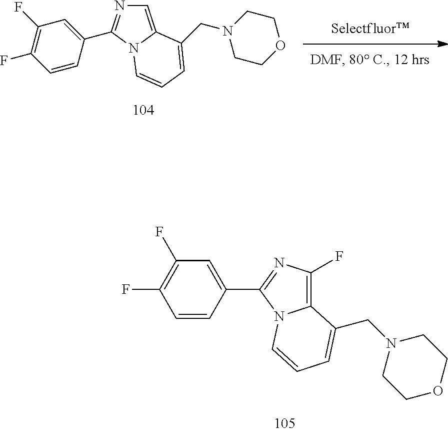 [SMILES: Fc1ccc(-c2ncc3c(CN4CCOCC4)cccn23)cc1F ; Fc1ccc(-c2nc(F)c3c(CN4CCOCC4)cccn23)cc1F]

Compound 104 (200 mg, 607 μmol, 1.00 eq) was dissolved in DMF (0.50 mL), and a selective fluorinating reagent (430 mg, 1.21 mmol, 2.00 eq) was added. The mixture was stirred at 80° C. for 12 hours. LC-MS analysis indicated the formation of the target compound (m/z=348.4, M+H⁺). The reaction mixture was filtered and concentrated under reduced pressure to obtain a crude product, which was purified by reverse-phase HPLC (column: Waters xbridge 150×25 mm×10 μm; mobile phase: [water (NH₄HCO₃)-ACN]; gradient: 35%-65% B over 18 min). This resulted in a yellow solid compound 105 (4.36 mg, 28.2 μmol), yielding 4.65% with a purity of 98.1%.

LCMS: m/z=348.4, M+H⁺

¹H NMR: (400 MHz, MeOD) δ 8.21 (d, J=7.6 Hz, 1H), 7.66-7.72 (m, 1H), 7.57-7.61 (m, 1H), 7.42-7.49 (m, 1H), 6.76 (d, J=6.4 Hz, 1H), 6.69 (t, J=7.2 Hz, 1H), 3.69-3.71 (m, 6H), 2.56-2.58 (m, 4H).

### Biological Activity Data

### Example 24: Binding Affinity Testing

### (1) Preparation of Sigma-1 Receptor Membranes

Guinea pigs (provided by Beijing Vital River Laboratory Animal Technology Co., Ltd.) were decapitated, and the operations were carried out on ice. The cerebral cortex was quickly harvested and pooled into a centrifuge tube. A suitable amount of buffer (0.05M Tris-HCl with 0.32M sucrose) was added to the tube, and the tissue was homogenized using a homogenizer (IKA, Ultra Turrax T25 Digital) at setting 4 for 3˜4 seconds, repeated four times. After homogenization, solution B was added to adjust the volume to 10 mL/g. The weight of the centrifuge tubes was balanced, and then they were centrifuged at 1000 g for 10 minutes using a high-speed refrigerated centrifuge. The supernatant was collected and adjusted to 2 mL/g with solution B, followed by centrifugation at 1000 g and 4° C. for 10 minutes. The supernatant was then centrifuged at 18000 rpm and 4° C. for 25 minutes. The pellet was resuspended in solution B to a final volume of 3 mL/g and incubated in a water bath at 25° C. for 15 minutes. After incubation, the suspension was centrifuged at 18000 rpm and 4° C. for 45 minutes. The pellet was stored at −80° C. for future use.

### (2) Experimental Process of Binding Affinity Test

An appropriate amount of homogenization buffer was added to the prepared membrane, and was dispersed into a suspension using a homogenizer, protein concentration was measured to be 4 mg/ml. 100 micrograms of protein were added to each well of a 96-well plate, with a volume of 90 μl. 1 μl of the compound was added to the test wells (the highest final concentration is 10 μM, diluted fourfold, across 10 concentrations), followed by 1 μl buffer to the HPE wells, and 1 μl of haloperidol (MedChemExpress, Cat #HY-14538) to the HPE wells (final concentration of 1 μM). [3H]-(+)-pentazocine (Perkin-Elmer, Cat #NET1056250UC) was added to each well (final concentration of 10 nM). The 96-well plate was incubated in a constant temperature water bath (25° C., 180 minutes). After incubation, the suspension was quickly filtered in the 96 wells through a GF/C plate prepared in advance with 0.25% PEI solution using vacuum filtration, followed by washing the GF/C three times with assay buffer. After washing, the samples were dried in a 37° C. oven. 50 μl/well of scintillation fluid (Perkin Elmer, Cat #6013621) was added to the GF/C plate. The GF/C plate was placed into a liquid scintillation counter (Perkin Elmer 1450 MicroBeta TriLux) and operated according to the program to read the experimental values.

### (3) Experimental Results

TABLE 1
Affinity of compounds to Sigma-1 receptors.

| Compound | Sigma1R Ki(nM) |
|---|---|
| 1 | 80.33 |
| 19 | 171.6 |
| 20 | 48.93 |
| 37 | 308.2 |
| 38 | 378.50 |
| 40 | 190.00 |
| 41 | 34.02 |
| 43 | 7.97 |
| 44 | 18.99 |
| 47 | 0.74 |
| 65 | 39.57 |
| 74 | 84.44 |
| 77 | 14.86 |
| 88 | 958.89 |
| Reference | 6.17 |
| compound 1 | |
| Reference | 313.3 |
| compound 2 | |
| haloperidol | 5.037 |

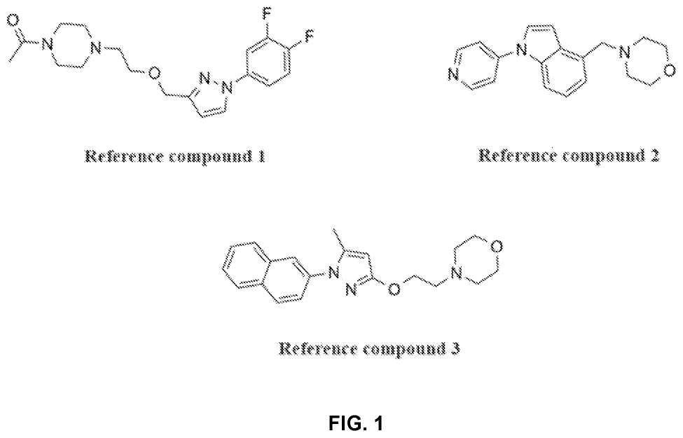

Note:
Refer to the structures of reference compounds 1-2 as shown in FIG. 1.

### Example 25: SNL Model

Neuropathic pain is pain directly caused by damage or disease of the somatosensory nervous system. It is a type of chronic pain characterized by features such as spontaneous pain, hyperalgesia, abnormal pain, and sensory disturbances. Following nerve damage, neuropathic pain can persist, and its characteristics include hypersensitive responses to stimuli (hyperalgesia), the presence of abnormal sensations (allodynia), and painful responses to non-harmful stimuli (hyperalgesia). The Spinal Nerve Ligation (SNL) model involves the tight ligation of the L5 and L6 spinal nerves using sutures. After the surgical modeling, animals exhibit clear signs of neuropathic pain, such as mechanical allodynia, in the ipsilateral hind paw, which appears 7-14 days post-surgery.

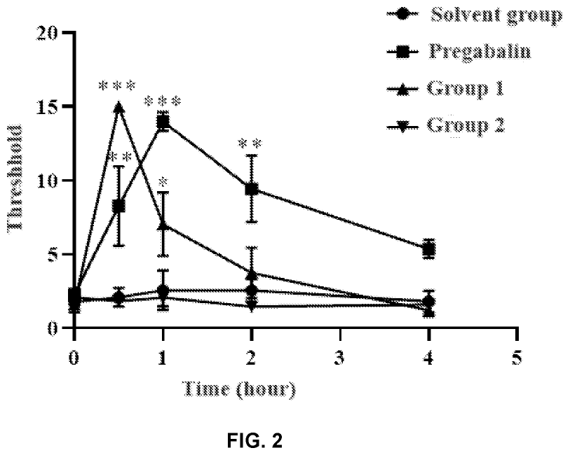

In this experiment, pregabalin at a dose of 100 mg/kg was used as a positive control to compare the analgesic effect of the test compound. After administration, the mechanical pain threshold in the ipsilateral hind paw of the rat was tested using Von Frey filaments. The change in mechanical pain threshold before and after administration was used to determine whether the test compound at 100 mg/kg has a neuropathic pain-relieving effect in the SNL model. The experimental results are shown in FIG. 2. In the rat SNL model, oral administration of compound 37 at 100 mg/kg and pregabalin exhibited some analgesic activity, while oral administration of the reference compound 3 at 10 mg/kg showed no analgesic activity in the SNL model.

TABLE 2
Analgesic effects of compounds in the SNL model.

| | | Dosage | Dosing |
|---|---|---|---|
| Group | Drug | (mg/kg) | method |
| Solvent group | 0.9% Sodium Chloride Injection | — | — |
| 1 | Compound 37 | 100 | i.g |
| 2 | Reference Compound 3 | 100 | i.g |
| 3 | Pregabalin | 100 | i.g |

Note:
The structure of reference compound 3 is shown in FIG. 1.

### Example 26: Rat Rotarod Test

Placing rats on a continuously rotating rod causes them to move immediately in the opposite direction of the rod's motion. Based on this characteristic, rats are initially placed on a steadily rotating rotarod for adaptation training, and those rats demonstrating satisfactory motor balance skills are selected for the actual test. During the formal experiment, the time the rats stay on the rotarod is used as an indicator to measure their motor balance ability. This experiment aims to explore the impact of compound 37 at doses ranging from 10 to 300 mg/kg on the motor balance ability of rats in the rotarod test.

After administration of the test substance to the rats, they are placed on the rotarod fatigue tester, and the average time spent on the rod after three tests is recorded and analyzed.

Calculate the Maximum Possible Effect on Motor Balance Ability %: Maximum Possible Effect on Motor Balance Ability %=(1−test group/vehicle)×100%

Note: The motor balance ability of the vehicle group is considered as 0%, and the Maximum Possible Effect on Motor Balance Ability % for each group is calculated based on this standard.

In the rat rotarod test, the time spent on the rod by the rats at a rotation speed of 15 rpm increases with the training time, indicating that the experimental system is functioning normally. Compared to the vehicle, subcutaneous injection of 30 μg/kg dexmedetomidine hydrochloride injection 15 minutes prior results in a Maximum Possible Effect on Motor Balance Ability of 62% (P<0.001), which is consistent with historical results, suggesting the reliability and effectiveness of the experimental procedure. In contrast, when compound 37 is administered orally 15 minutes prior at doses of 10-300 mg/kg, there is no significant change in the time spent on the rotarod compared to the vehicle, with Maximum Possible Effects on Motor Balance Ability of −6%, 11%, −6%, and −6%, respectively, indicating that compound 37 at doses of 10-300 mg/kg (i.g.) has no impact on the motor balance ability of rats.

What is claimed is:

1. A compound as shown in formula (I), or its stereoisomers, pharmaceutically acceptable salts, solvates, deuterated derivatives, metabolites, or prodrugs; 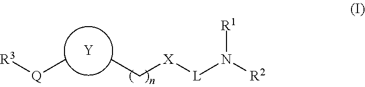
wherein,
n=0, 1, 2, or 3;
L is selected form the group consisting of —(CH₂)ₘ—, —(CH₂)ₘ(CR⁵R⁶)—, and —(CH₂)ₘQ-;
m=0, 1, 2, or 3;
X is C, O, or S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl;
wherein the substituted C1-C6 alkyl, substituted C3-C6 cycloalkyl, substituted aryl, substituted heteroaryl are substituted by 1-3 substituents independently selected from the following groups: halogen, C1-C3 alkyl, C1-C3 alkoxy, halogenated C1-C3 alkyl, heteroaryl, heterocycle;
alternatively, R¹ and R² together with the nitrogen atom to which they are attached form a substituted or unsubstituted heterocyclic group, with representative structures comprising: 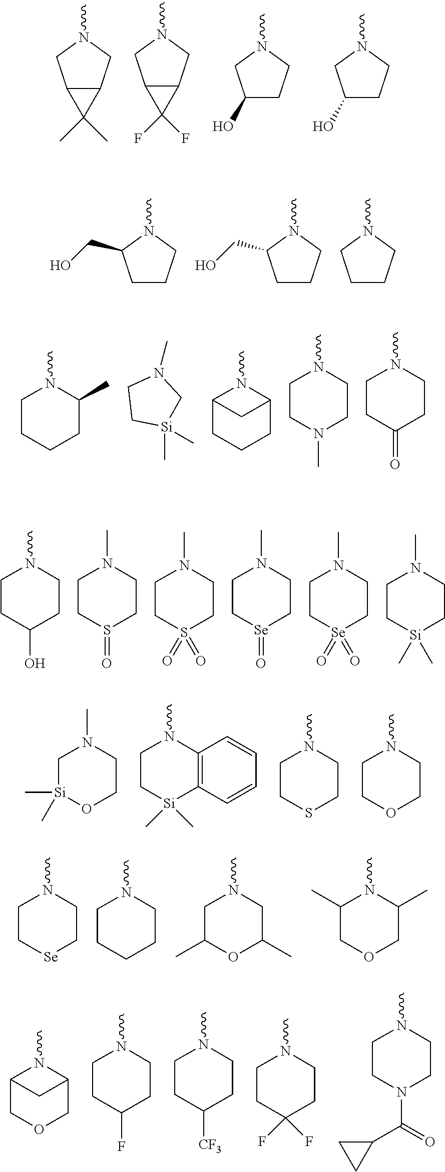 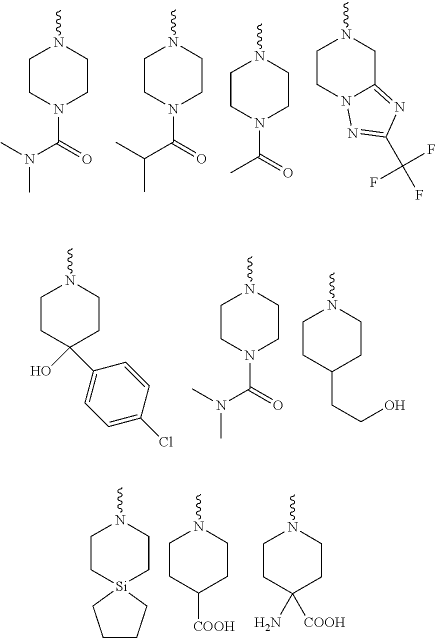
Q is phenyl or heteroaryl groups,
wherein the phenyl or heteroaryl groups are optionally further substituted by 0-5 R³ groups;
R³ is selected from the group consisting of halogens, C1-C6 alkyl groups, and C3-C6 cycloalkyl groups;
wherein, Y is C5-C14 heteroaryl, and the C5-C14 heteroaryl is optionally further substituted by 0-5 R⁴ groups; the C5-C14 heteroaryl contain 1-4 heteroatoms selected from N, O, or S;
R⁴ is selected from the group consisting of hydrogen, halogens, cyano, C1-C6 alkyl groups, substituted C1-C6 alkyl groups, C3-C6 cycloalkyl groups, substituted C3-C6 cycloalkyl groups, aryl groups, substituted aryl groups, heterocycles, and substituted heterocycles;
wherein the substituted C1-C6 alkyl, substituted C3-C6 cycloalkyl, substituted aryl, and substituted heteroaryl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are independently selected from the group consisting of hydrogen, C1-C6 alkyl, substituted C1-C6 alkyl, C3-C6 cycloalkyl, and substituted C3-C6 cycloalkyl;
wherein the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

2. The compound of claim 1, wherein the compound is represented by formula (II), or its stereoisomers, pharmaceutically acceptable salts, solvates, deuterated forms, metabolites, or prodrugs. 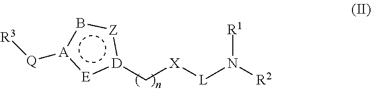
wherein,
n=0, 1, 2, 3;
L is selected from —(CH₂)ₘ—, —(CH₂)ₘ(CR⁵R⁶)—, —(CH₂)ₘQ-;
m=0, 1, 2, 3;
X is selected from C, O, S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, and substituted heteroaryl;
wherein the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle;
alternatively, R¹ and R² together with the nitrogen atom to which they are attached may form a substituted or unsubstituted heterocyclic group, as shown in claim 1;
Q is aryl or heteroaryl, wherein the aryl or heteroaryl are optionally further substituted by 0-5 R³ groups;
R³ is a halogen, C1-C6 alkyl, or C3-C6 cycloalkyl;
A, B, D, E, Z are each independently C, N, or O;
wherein, when any one of B, Z, E is selected from C, it can be connected to R⁴;
R⁴ is selected from hydrogen, halogens, cyano, C1-C6 alkyl, substituted C1-C6 alkyl, C3-C6 cycloalkyl, substituted C3-C6 cycloalkyl, aryl, substituted aryl, heterocycle, substituted heterocycle; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine.

3. The compound of claim 2, wherein the compound is represented by formula (IIa), or its stereoisomers, pharmaceutically acceptable salts, solvates, deuterated forms, metabolites, or prodrugs; 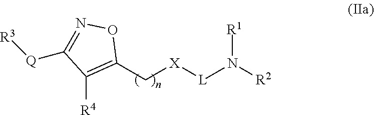
wherein
n=0 or 1;
L is —(CH₂)ₘ—, —(CH₂)ₘ(CR⁵R⁶)—, or —(CH₂)ₘQ-;
m=0, 1, or 2;
X is C, O, or S;
R¹ and R² are each independently selected from the group consisting of hydrogen, C1-C6 alkyl, substituted C1-C6 alkyl, C3-C6 cycloalkyl, substituted C3-C6 cycloalkyl, aryl, substituted aryl, heteroaryl, substituted heteroaryl; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle;
alternatively, R¹ and R² together with the nitrogen atom to which they are attached may form a substituted or unsubstituted heterocyclic group, as shown in claim 1;
Q is aryl or heteroaryl, wherein the aryl or heteroaryl is optionally further substituted by 0-5 R³ groups;
R³ is halogens, C1-C6 alkyl, or C3-C6 cycloalkyl;
R⁴ is selected from the group consisting of hydrogen, halogens, cyano, C1-C6 alkyl, substituted C1-C6 alkyl, C3-C6 cycloalkyl, substituted C3-C6 cycloalkyl, aryl, substituted aryl, heterocycle, and substituted heterocycle; the substituted C1-C6 alkyl, substituted C3-C6 cycloalkyl, substituted aryl, substituted heteroaryl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine.

4. The compound of claim 3, wherein the compound is represented by formulas (IIa-1), (IIa-2), (IIa-1a), (IIa-1 b), and (IIa-1c), or their stereoisomers, pharmaceutically acceptable salts, solvates, deuterated forms, metabolites, or prodrugs; when X is O, n=1, as shown in formula (IIa-1): 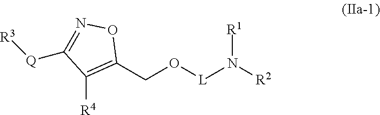
wherein, L is —(CH₂)ₘ—, —(CH₂)ₘ(CR⁵R⁶)—, —(CH₂)ₘQ-, as shown in formulas (IIa-1a), (IIa-1b), and (IIa-1c): 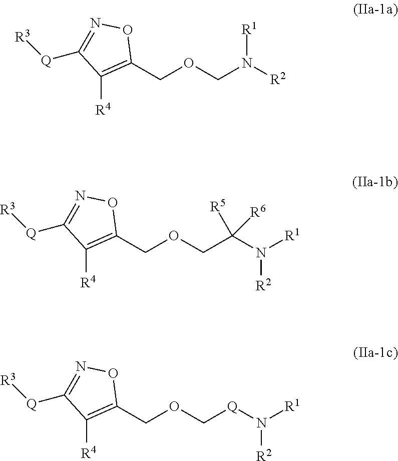
when X is C, n=0, and L is —(CH₂)ₘ— where m=0, as shown in formula (IIa-2): 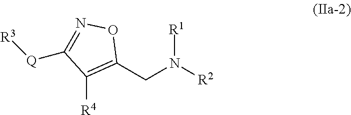
R¹ and R² are each independently selected from the group consisting of hydrogen, methyl, ethyl, propyl, isopropyl, butyl, cyclopropyl, cyclobutyl, cyclopentyl, cyclohexyl, phenyl, naphthyl, and pyridine, the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine;
alternatively, R¹ and R² together with the nitrogen atom to which they are attached may form a substituted or unsubstituted heterocyclic group, as shown in claim 1;
Q is selected from the group consisting of phenyl, naphthyl, pyrrolyl, pyrazolyl, pyridyl, quinolyl, pyrazinyl, and optionally further substituted by 0-5 R³ groups;
R³ is selected from the group consisting of fluorine, chlorine, bromine, methyl, ethyl, propyl, isopropyl, butyl, tert-butyl, cyclopropyl, cyclobutyl, cyclopentyl, and cyclohexyl;
R⁴ is selected from the group consisting of hydrogen, fluorine, chlorine, bromine, cyano, methyl, ethyl, propyl, isopropyl, butyl, tert-butyl, cyclopropyl, cyclobutyl, cyclopentyl, cyclohexyl, phenyl, naphthyl, and pyridine; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine; and
R⁵ and R⁶ are each independently selected from the group consisting of hydrogen, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl, substituted phenyl, substituted heteroaryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

5. The compound of claim 2, a compound as represented by formula (IIb), or its stereoisomers, pharmaceutically acceptable salts, solvates, deuterated forms, metabolites, or prodrugs; 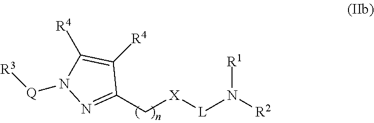
wherein,
n=0 or 1;
L is —(CH₂)ₘ—, —(CH₂)ₘ(CR⁵R⁶)—, or —(CH₂)ₘQ-;
m=0, 1, or 2;
X is C, O, or S;
R¹ and R² are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl are substituted by 1-3 substituents independently selected from the following groups: halogens, C1-C3 alkyl, C1-C3 alkoxy, halogenated C1-C3 alkyl, heteroaryl, heterocycle;
alternatively, R¹ and R² together with the nitrogen atom to which they are attached may form a substituted or unsubstituted heterocyclic group, as shown in claim 1;
Q is aryl or heteroaryl, wherein the aryl, heteroaryl are optionally further substituted by 0-5 R³ groups;
R³ is selected from halogens, C1-C6 alkyl, C3-C6 cycloalkyl;
R⁴ is selected from hydrogen, halogens, cyano, C1-C6 alkyl, substituted C1-C6 alkyl, C3-C6 cycloalkyl, substituted C3-C6 cycloalkyl; the substituted C1-C6 alkyl, substituted C3-C6 cycloalkyl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from hydrogen, C1-C6 alkyl, substituted C1-C6 alkyl, C3-C6 cycloalkyl, substituted C3-C6 cycloalkyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine.

6. The compound of claim 4, wherein the compound is represented by formulas (IIb-1) and (IIb-2), or their stereoisomers, pharmaceutically acceptable salts, solvates, deuterated forms, metabolites, or prodrugs; 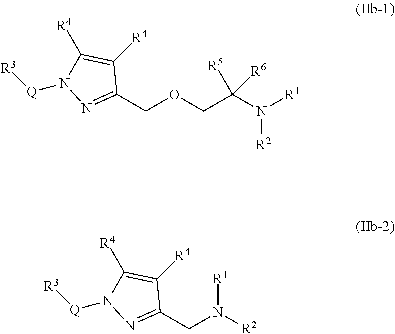
R¹ and R² are each independently selected from the group consisting of hydrogen, methyl, ethyl, propyl, isopropyl, butyl, cyclopropyl, cyclobutyl, cyclopentyl, cyclohexyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, and piperazine;
alternatively, R¹ and R² together with the nitrogen atom to which they are attached may form a substituted or unsubstituted heterocyclic group, as shown in claim 1;
Q is selected from the group consisting of phenyl, naphthyl, pyrrolyl, pyrazolyl, pyridyl, quinolyl, pyrazinyl, and optionally further substituted by 0-5 R³ groups;
R³ is selected from the group consisting of fluorine, chlorine, bromine, methyl, ethyl, propyl, isopropyl, butyl, tert-butyl, cyclopropyl, cyclobutyl, cyclopentyl, and cyclohexyl;
R⁴ is selected from the group consisting of hydrogen, fluorine, chlorine, bromine, cyano, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl are substituted by 1-3 substituents independently selected from the following groups: fluorine, chlorine, bromine, methyl, ethyl, propyl, methoxy, ethoxy, trifluoromethyl, pyridine, piperidine, piperazine; and
R⁵ and R⁶ are each independently selected from the group consisting of hydrogen, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl are substituted by 1-3 substituents independently selected from the group consisting of fluorine, chlorine, bromine, methyl, ethyl, propyl, methoxy, ethoxy, trifluoromethyl, pyridine, piperidine, and piperazine.

7. The compound of claim 1, wherein the compound is represented by formula (III), or its stereoisomers, pharmaceutically acceptable salts, solvates, deuterated forms, metabolites, or prodrugs; 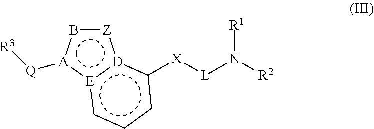
wherein,
L is —(CH₂)ₘ—, —(CH₂)ₘ(CR⁵R⁶)—, or —(CH₂)ₘQ-;
m=0, 1, or 2;
X is C, O, or S;
R¹ and R² are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl are substituted by 1-3 substituents independently selected from the group consisting of halogens, C1-C3 alkyl, C1-C3 alkoxy, halogenated C1-C3 alkyl, heteroaryl, and heterocycle;
alternatively, R¹ and R² together with the nitrogen atom to which they are attached may form a substituted or unsubstituted heterocyclic group, as shown in claim 1;
Q is aryl or heteroaryl, and the aryl or heteroaryl are optionally further substituted by 0-5 R³ groups;
R³ is halogens, C1-C6 alkyl, or C3-C6 cycloalkyl;
A, B, D, E, Z are each independently C, N, or O;
wherein, when any one of B, Z, E is selected from C, it can be connected to R⁴;
R⁴ is selected from the group consisting of hydrogen, halogens, cyano, C1-C6 alkyl, substituted C1-C6 alkyl, C3-C6 cycloalkyl, and substituted C3-C6 cycloalkyl; the substituted C1-C6 alkyl, substituted C3-C6 cycloalkyl are substituted by 1-3 substituents independently selected from the following groups: halogens, C1-C3 alkyl, C1-C3 alkoxy, halogenated C1-C3 alkyl, heteroaryl, heterocycle; and
R⁵ and R⁶ are each independently selected from the group consisting of hydrogen, C1-C6 alkyl, substituted C1-C6 alkyl, C3-C6 cycloalkyl, and substituted C3-C6 cycloalkyl; the substituted C1-C6 alkyl, substituted C3-C6 cycloalkyl, substituted phenyl, substituted heteroaryl are substituted by 1-3 substituents independently selected from the following groups: fluorine, chlorine, bromine, methyl, ethyl, propyl, methoxy, ethoxy, trifluoromethyl, pyridine, piperidine, piperazine.

8. The compound of claim 7, wherein the compound is represented by formulas (IIIa), (IIIb), (IIIc), (IIId), or their stereoisomers, pharmaceutically acceptable salts, solvates, deuterated forms, metabolites, or prodrugs; 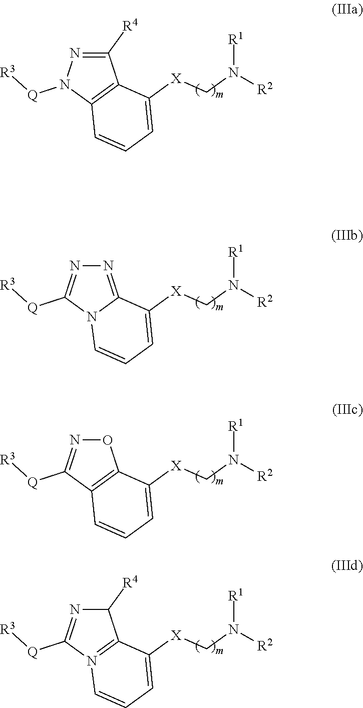
wherein
m=0, 1, 2, or 3;
X is C, O, or S;
R¹ and R² are each independently selected from the group consisting of hydrogen, methyl, ethyl, propyl, isopropyl, butyl, cyclopropyl, cyclobutyl, cyclopentyl, and cyclohexyl; the substituted C1-C6 alkyl, substituted C3-C6 cycloalkyl are substituted by 1-3 substituents independently selected from the group consisting of fluorine, chlorine, bromine, methyl, ethyl, propyl, methoxy, ethoxy, trifluoromethyl, pyridine, piperidine, and piperazine;
alternatively, R¹ and R² together with the nitrogen atom to which they are attached may form a substituted or unsubstituted heterocyclic group, as shown in claim 1;
Q is selected from the group consisting of phenyl, naphthyl, pyrrolyl, pyrazolyl, pyridyl, quinolyl, and pyrazinyl, and optionally further substituted by 0-5 R³ groups;
R³ is selected from fluorine, chlorine, bromine, methyl, ethyl, propyl, isopropyl, butyl, tert-butyl, cyclopropyl, cyclobutyl, cyclopentyl, cyclohexyl; and
R⁴ is selected from the group consisting of hydrogen, fluorine, chlorine, bromine, cyano, methyl, ethyl, propyl, isopropyl, butyl, tert-butyl, cyclopropyl, cyclobutyl, cyclopentyl, and cyclohexyl; the substituted C1-C6 alkyl, substituted C3-C6 cycloalkyl are substituted by 1-3 substituents independently selected from the following groups: fluorine, chlorine, bromine, methyl, ethyl, propyl, methoxy, ethoxy, trifluoromethyl, pyridine, piperidine, piperazine.

9. The compound of claim 2, wherein the compound of formula (II) comprises of following compounds or any pharmaceutically acceptable salt thereof:

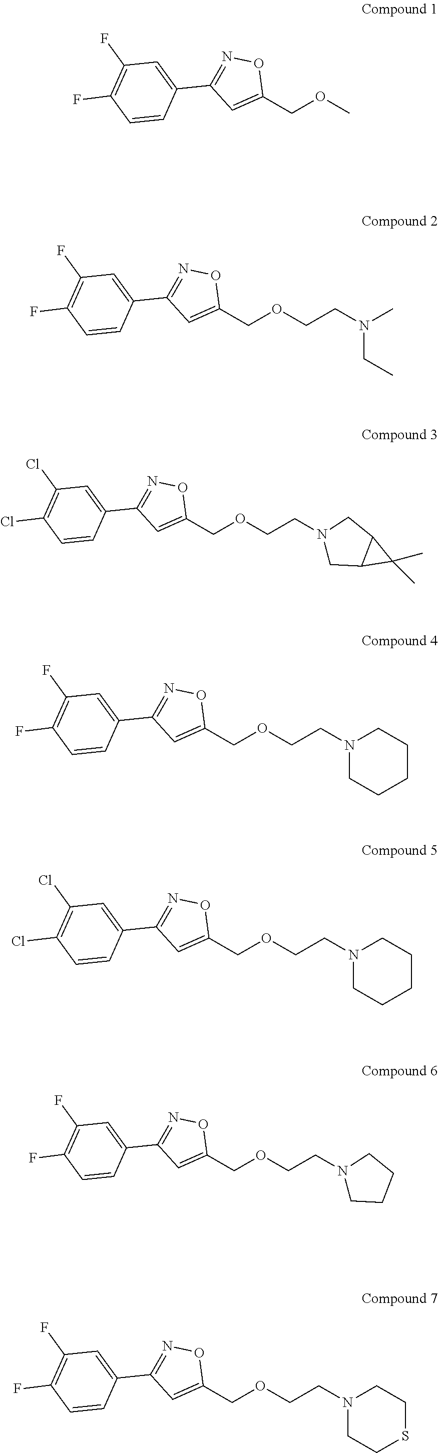

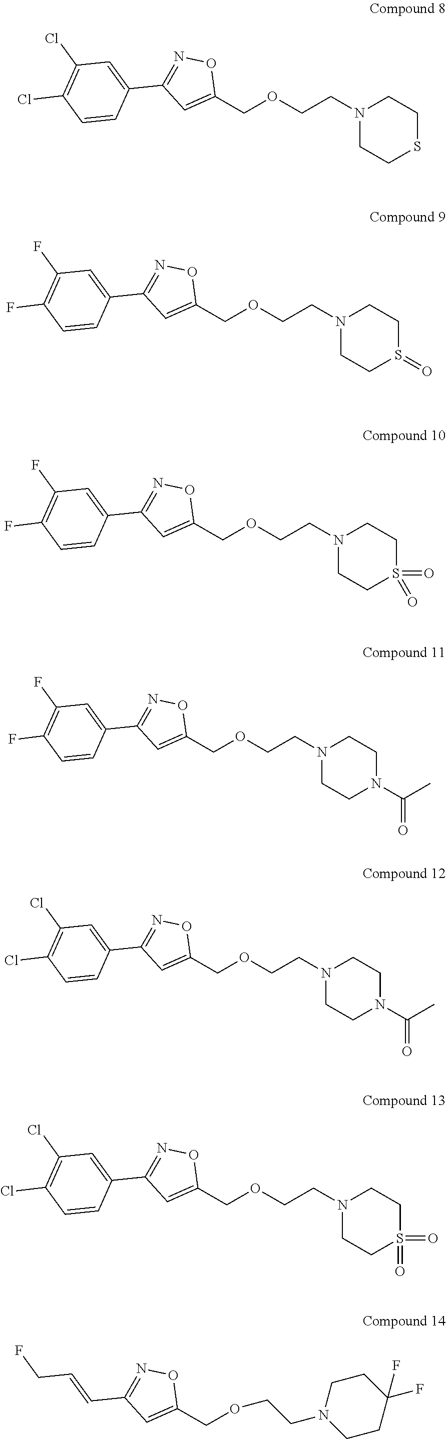

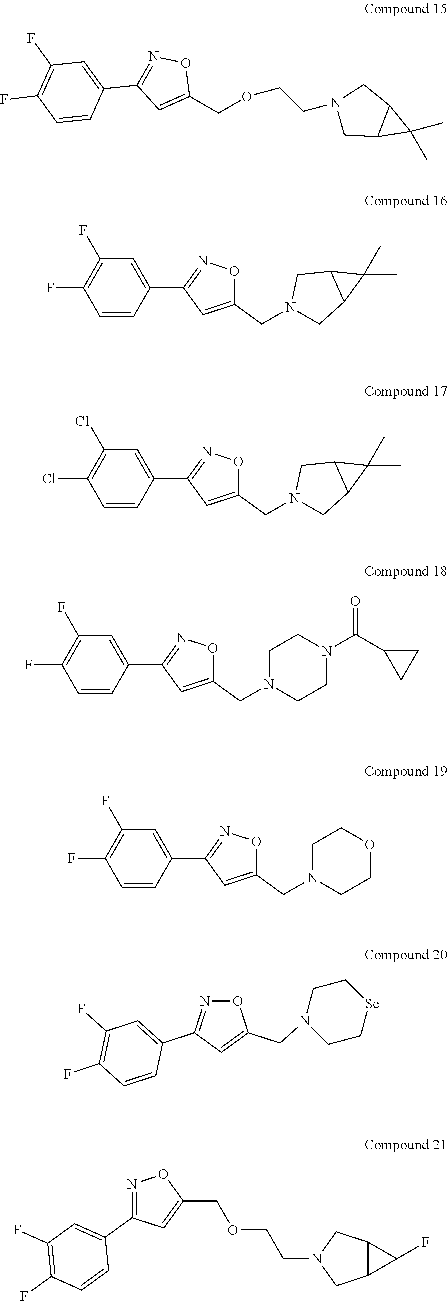

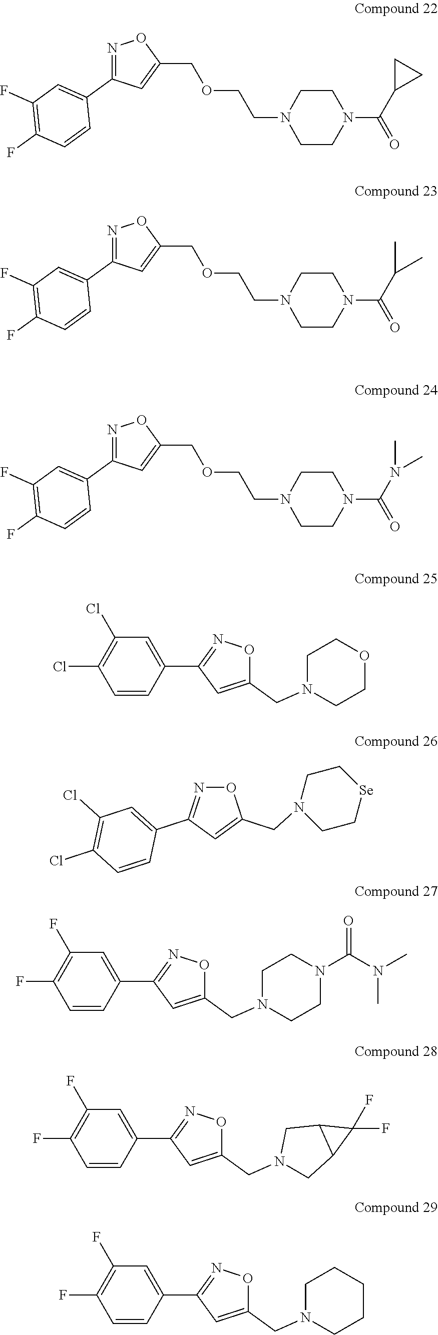

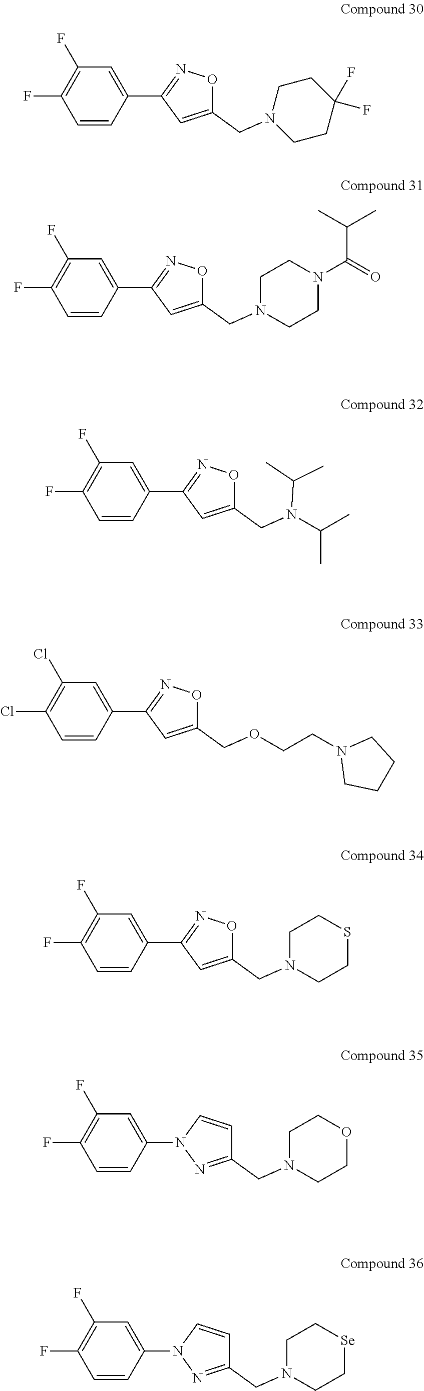

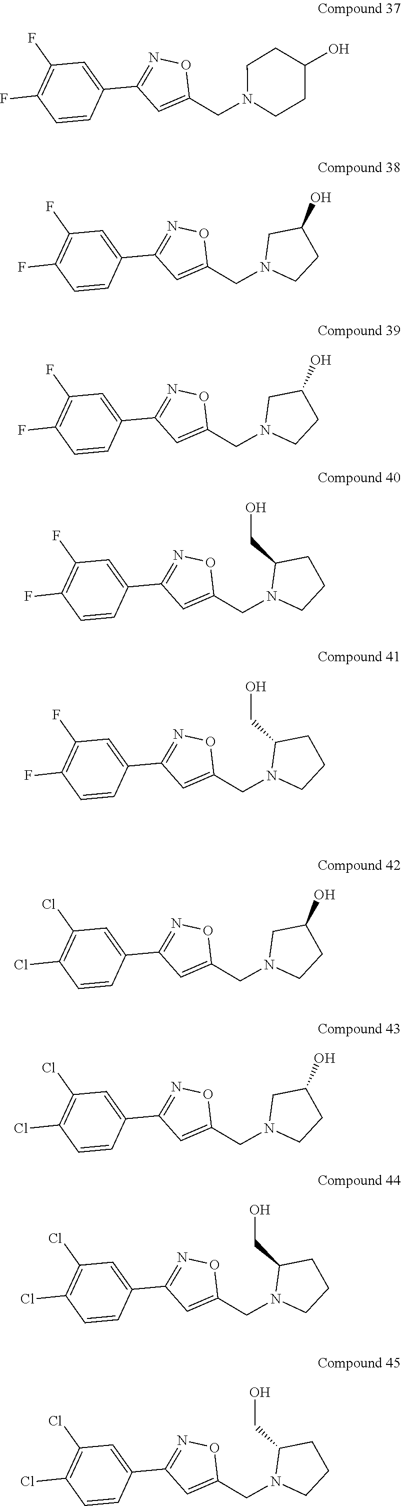

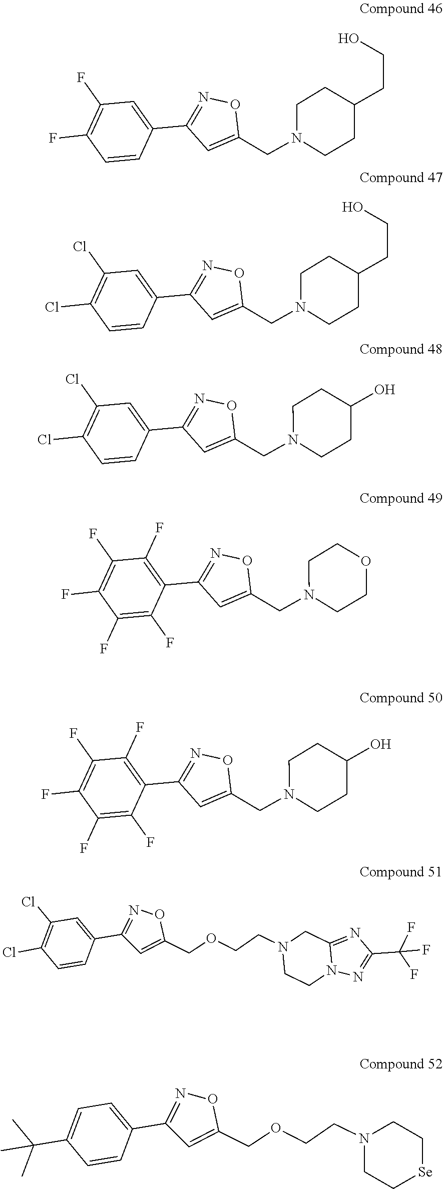

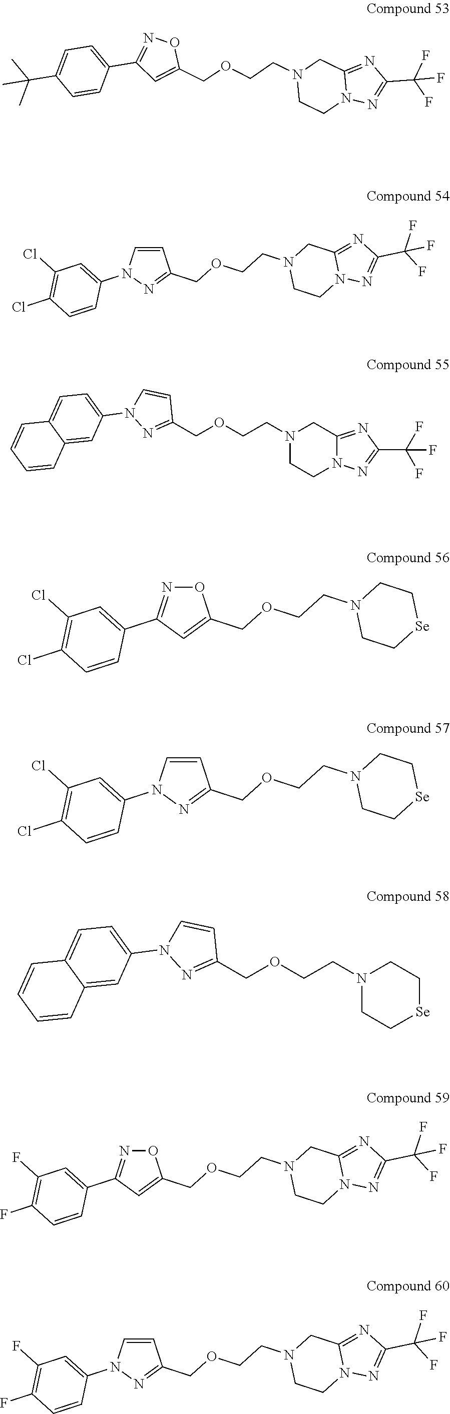

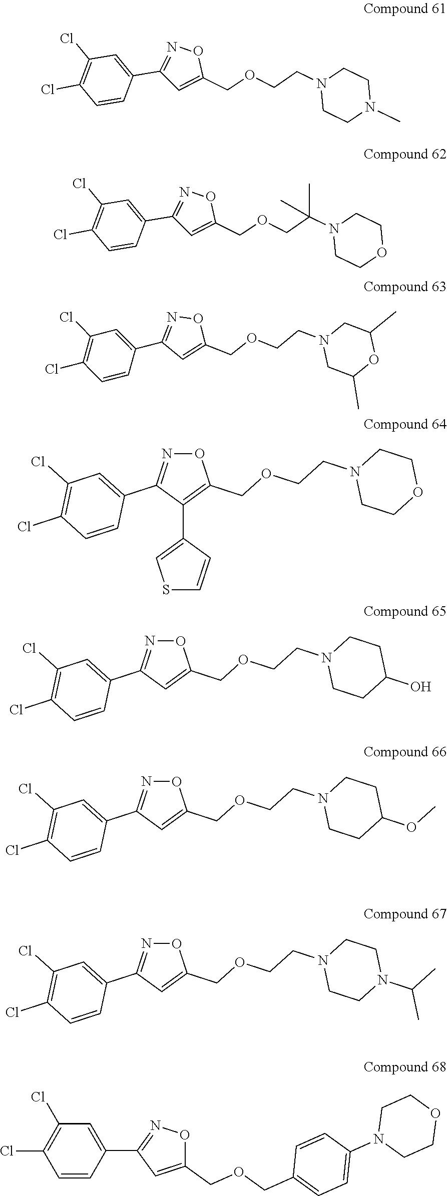

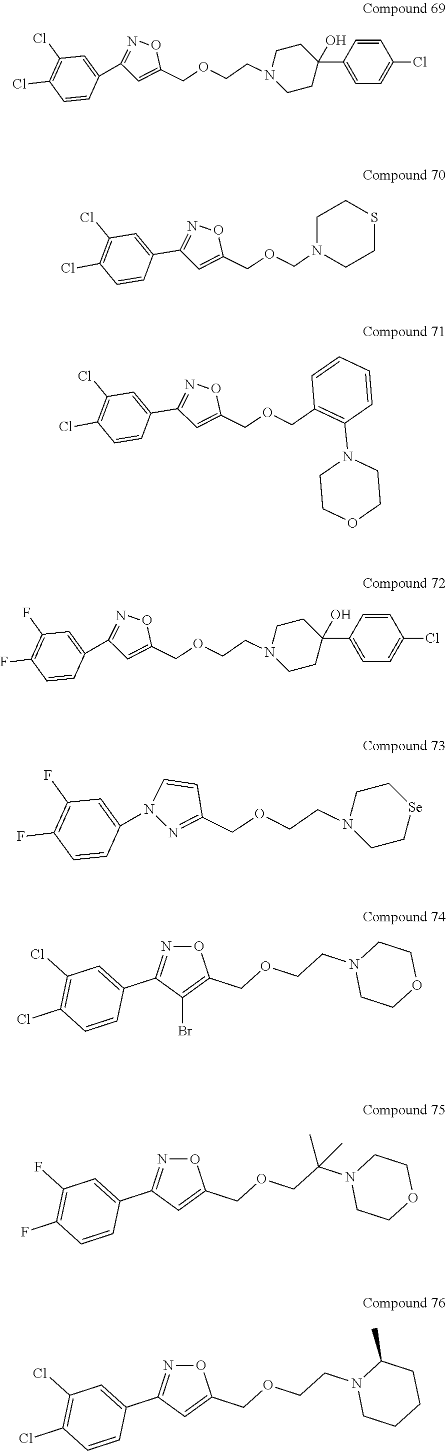

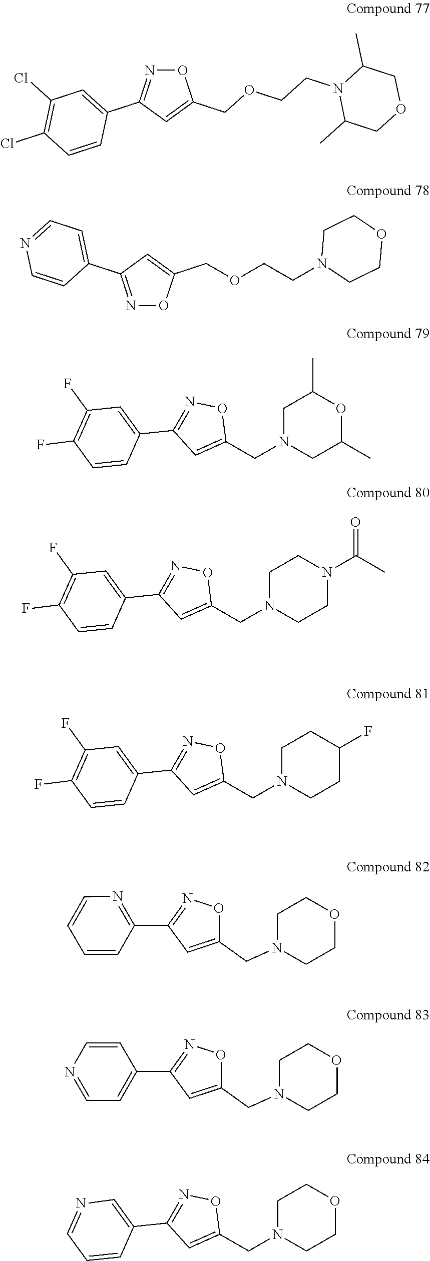

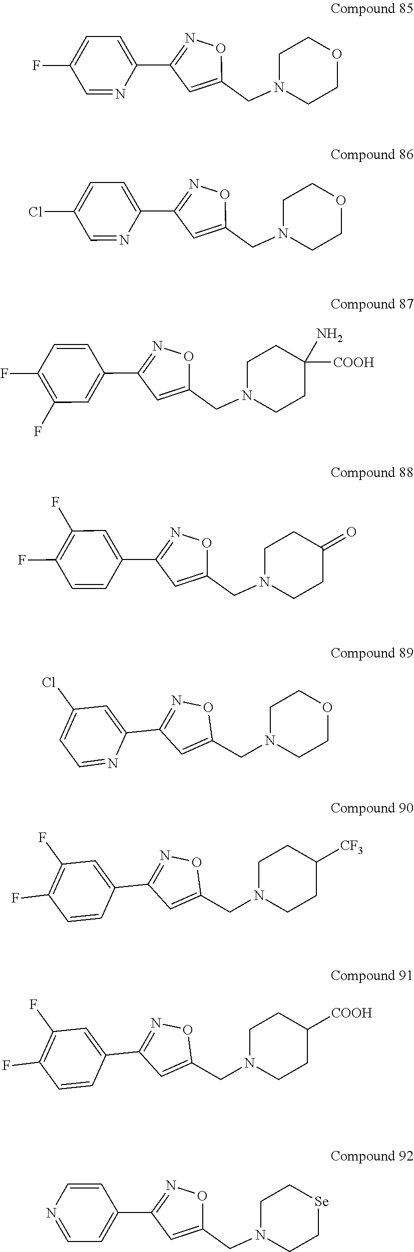

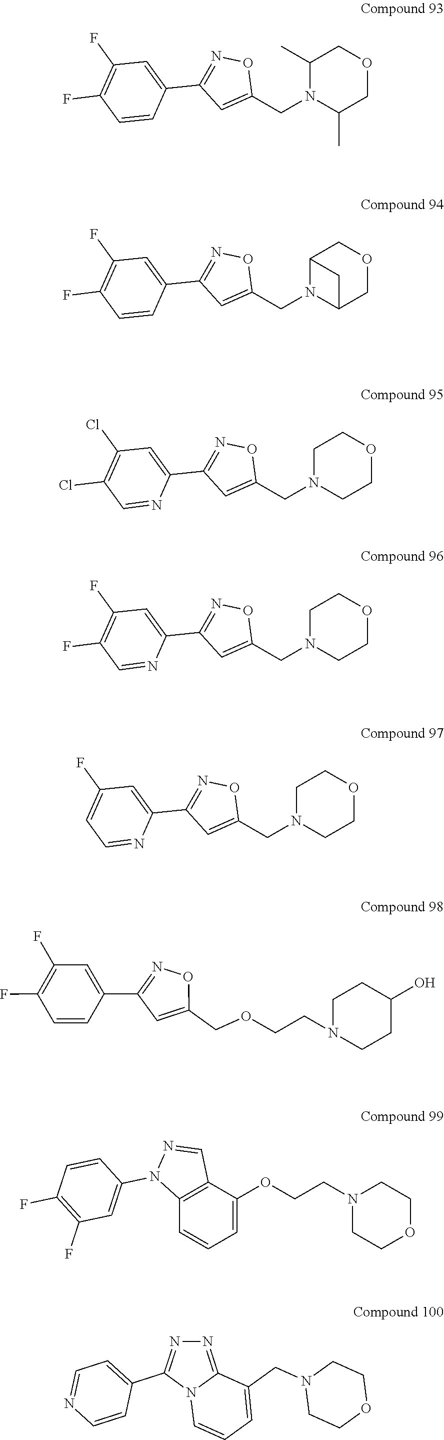

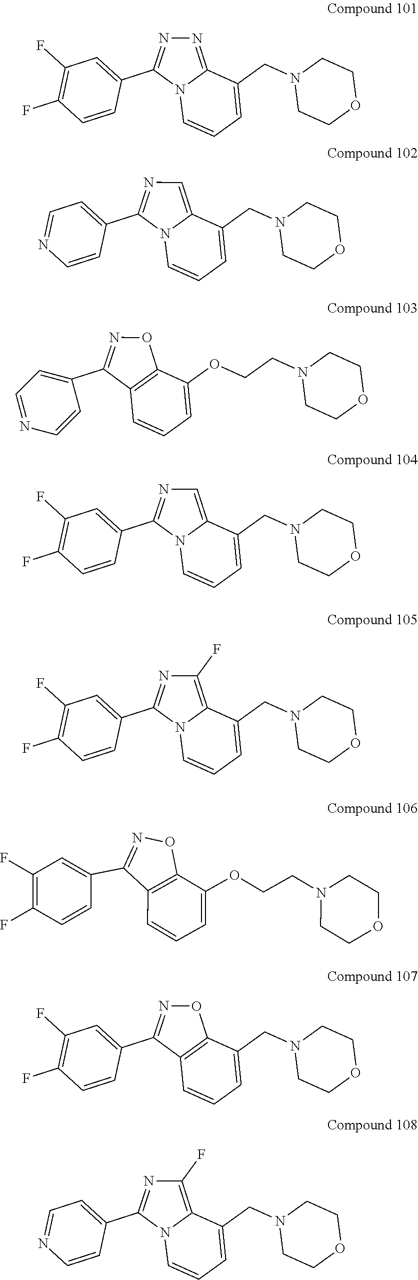

10. The compound of claim 7, wherein the compound of formula (III) is selected from the group consisting of following compounds or any pharmaceutically acceptable salt thereof:

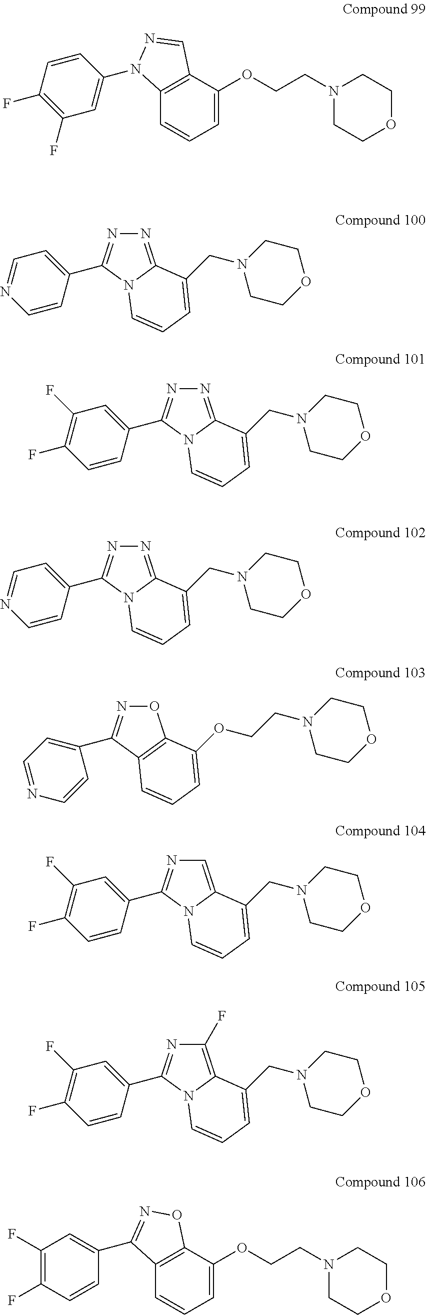

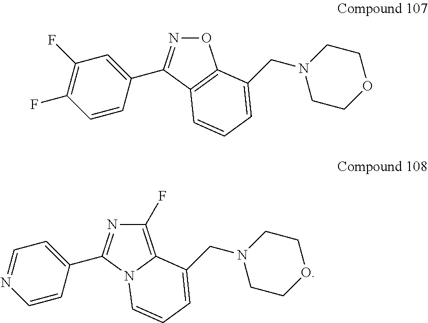 [SMILES: Fc1ccc(-c2noc3c(CN4CCOCC4)cccc23)cc1F ; Fc1nc(-c2ccncc2)n2cccc(CN3CCOCC3)c12]

11. A pharmaceutical composition comprising a compound of claim 1, or its stereoisomers, pharmaceutically acceptable salts, solvates, deuterated forms, metabolites, or prodrugs, and a pharmaceutically acceptable carrier or excipient.

12. A method to inhibit a Sigma-1 receptor in a subject, the method comprising administering a compound of claim 1 to the subject.

13. A method to treat and/or prevent diseases or disorders related to a Sigma-1 receptor in a subject, the method comprising administering a compound of claim 1 to the subject.

14. A method to treat and/or prevent conditions such as pain, the method comprising administering a compound of claim 1 to the subject.

15. A method to treat and/or prevent one or more condition related to a Sigma-1 receptor in a subject, the method comprising administering a compound of claim 1 to the subject, wherein the one or more condition comprises pain, psychosis, substance abuse, or cancer.
